(19) United States
(12) Patent Application Publication
(10) Pub. No.: US 2010/0136097 A1
(43) Pub. Date: Jun. 3, 2010

(54) Systems for modulating inflammation

(75) Inventors: Roderick A. Hyde, Redmond, WA (US); Stephen L. Malaska, Redmond, WA (US); Elizabeth A. Sweeney, Seattle, WA (US); Lowell L. Wood, JR., Bellevue, WA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 12/315,514

(22) Filed: Dec. 2, 2008

(51) Int. Cl. A61K 9/127; A61K 31/439; A61K 31/506; A61K 31/4706; A61K 31/145; A61K 31/69; A61K 31/7088; A61K 38/02; A61K 31/739; A61K 31/70

(52) U.S. Cl. 424/450; 514/305; 514/252.19; 514/313; 514/252.18; 514/275; 514/483; 514/64; 514/2; 514/8; 514/54; 514/23; 514/44 R

(57) ABSTRACT

Certain embodiments disclosed relate to compositions, including therapeutic compositions, methods, devices, and systems that modulate at least one inflammatory response or reaction. According to various embodiments, the compositions, methods, devices, and systems relate to modulating one or more of Toll-like receptors, Src family kinases, NF-kB molecules, proteases, or proteasomes.

### CROSS-REFERENCE TO RELATED APPLICATIONS

The present application is related to U.S. patent application Ser. No. to be assigned, entitled ANTI-INFLAMMATORY COMPOSITIONS AND METHODS, naming Roderick A. Hyde, Stephen L. Malaska, Elizabeth A. Sweeney and Lowell L. Wood, Jr. as inventors, filed 2 Dec. 2008, which is Docket No. 1207-004-001-000000.

The present application is related to U.S. patent application Ser. No. to be assigned, entitled DELIVERY DEVICES FOR MODULATING INFLAMMATION, naming Roderick A. Hyde, Stephen L. Malaska, Elizabeth A. Sweeney and Lowell L. Wood, Jr. as inventors, filed 2 Dec. 2008, which is Docket No. 1207-004-002-000000.

The present application is related to U.S. patent application Ser. No. to be assigned, entitled SYSTEMS FOR MODULATING INFLAMMATION, naming Roderick A. Hyde, Stephen L. Malaska, Elizabeth A. Sweeney and Lowell L. Wood, Jr. as inventors, filed 2 Dec. 2008, which is Docket No. 1207-004-003-000000.

The present application is related to U.S. patent application Ser. No. to be assigned, entitled ANTI-INFLAMMATORY COMPOSITIONS AND METHODS, naming Roderick A. Hyde, Stephen L. Malaska, Elizabeth A. Sweeney and Lowell L. Wood, Jr. as inventors, filed 2 Dec. 2008, which is Docket No. 1207-004-004-000000.

The present application is related to U.S. patent application Ser. No. to be assigned, entitled DELIVERY DEVICES FOR MODULATING INFLAMMATION, naming Roderick A. Hyde, Stephen L. Malaska, Elizabeth A. Sweeney and Lowell L. Wood, Jr. as inventors, filed 2 Dec. 2008, which is Docket No. 1207-004-005-000000.

The present application is related to U.S. patent application Ser. No. to be assigned, entitled SYSTEMS FOR MODULATING INFLAMMATION, naming Roderick A. Hyde, Stephen L. Malaska, Elizabeth A. Sweeney and Lowell L. Wood, Jr. as inventors, filed 2 Dec. 2008, which is Docket No. 1207-004-006-000000.

The present application is related to U.S. patent application Ser. No. to be assigned, entitled ANTI-INFLAMMATORY COMPOSITIONS AND METHODS, naming Roderick A. Hyde, Stephen L. Malaska, Elizabeth A. Sweeney and Lowell L. Wood, Jr. as inventors, filed 2 Dec. 2008, which is Docket No. 1207-004-007-000000.

The present application is related to U.S. patent application Ser. No. to be assigned, entitled DELIVERY DEVICES FOR MODULATING INFLAMMATION, naming Roderick A. Hyde, Stephen L. Malaska, Elizabeth A. Sweeney and Lowell L. Wood, Jr. as inventors, filed 2 Dec. 2008, which is Docket No.1207-004-008-000000.

The present application is related to U.S. patent application Ser. No. to be assigned, entitled ANTI-INFLAMMATORY COMPOSITIONS AND METHODS, naming Roderick A. Hyde, Stephen L. Malaska, Elizabeth A. Sweeney and Lowell L. Wood, Jr. as inventors, filed 2 Dec. 2008, which is Docket No. 1207-004-010-000000.

The present application is related to U.S. patent application Ser. No. to be assigned, entitled DELIVERY DEVICES FOR MODULATING INFLAMMATION, naming Roderick A. Hyde, Stephen L. Malaska, Elizabeth A. Sweeney and Lowell L. Wood, Jr. as inventors, filed 2 Dec. 2008, which is Docket No. 1207-004-011-000000.

The present application is related to U.S. patent application Ser. No. to be assigned, entitled SYSTEMS FOR MODULATING INFLAMMATION, naming Roderick A. Hyde, Stephen L. Malaska, Elizabeth A. Sweeney and Lowell L. Wood, Jr. as inventors, filed 2 Dec. 2008, which is Docket No. 1207-004-012-000000.

### SUMMARY

In one aspect, a composition for modulating cellular activity is described. In an embodiment, a composition includes at least one first agent configured to modulate the activity of one or more Toll-like receptors, and at least one second agent configured to modulate the activity of one or more Src family kinases. In an embodiment, a composition includes at least one third agent configured to modulate one or more NF-kB molecules or other transcription factors. In an embodiment, a composition includes at least one fourth agent configured to modulate the activity of at least one protease or proteasome.

In an embodiment, a composition includes at least one first agent configured to modulate the activity of one or more Toll-like receptors, and at least one second agent configured to modulate the activity of one or more NF-kB molecules or other transcription factors. In an embodiment, a composition includes at least one third agent configured to modulate one or more Src family kinases. In an embodiment, a composition includes at least one fourth agent configured to modulate the activity of at least one protease or proteasome.

In an embodiment, a composition includes at least one first agent configured to modulate the activity of one or more NF-kB molecules or other transcription factors, and at least one second agent configured to modulate one or more Src family kinases. In an embodiment, a composition includes at least one third agent configured to modulate one or more Toll-like receptors. In an embodiment, a composition includes at least one fourth agent configured to modulate the activity of at least one protease or proteasome.

In an embodiment, a therapeutic composition includes at least one first agent configured to modulate the activity of one or more Toll-like receptors, at least one second agent configured to modulate the activity of one or more Src family kinases, and at least one third agent configured to modulate one or more NF-kB molecules or other transcription factors. In an embodiment, a composition includes at least one fourth agent configured to modulate the activity of at least one protease or proteasome.

In an embodiment, the at least one first agent can be the same agent as one or more of the at least one second agent, the at least one third agent, or the at least one fourth agent. In an embodiment, the at least one second agent can be the same agent as one or more of the at least one first agent, the at least one third agent, or the at least one fourth agent. In an embodiment, the at least one third agent can be the same agent as one or more of the at least one first agent, the at least one second agent, or the at least one fourth agent. In an embodiment, the at least one fourth agent can be the same agent as one or more of the at least one first agent, the at least one second agent, or the at least one third agent.

In an embodiment, the at least one first agent can have similar kinetic reaction rates as one or more of the at least one second agent, the at least one third agent, or the at least one fourth agent. In an embodiment, the at least one second agent can have similar kinetic reaction rates as one or more of the at least one first agent, the at least one third agent, or the at least one fourth agent. In an embodiment, the at least one third agent can have similar kinetic reaction rates as one or more of the at least one first agent, the at least one second agnet, or the at least one fourth agent. In an embodiment, the at least one fourth agent can have similar kinetic reaction rates as one or more of the at least one first agent, the at least one second agent, or the at least one third agent.

In an embodiment, the at least one first agent can be different than one or more of the at least one second agent, the at least one third agent, or the at least one fourth agent. In an embodiment, the at least one second agent can be different than one or more of the at least one first agent, the at least one third agent, or the at least one fourth agent. In an embodiment, the at least one third agent can be different than one or more of the at least one first agent, the at least one second agent, or the at least one fourth agent. In an embodiment, the at least one fourth agent can be different than one or more of the at least one first agent, the at least one second agent, or the at least one third agent.

In an embodiment, one or more of the at least one first agent, or the at least one second agent, or the at least one third agent, or the at least one fourth agent, includes one or more of an organic or inorganic small molecule, nucleic acid, amino acid, peptide, polypeptide, protein, glycoprotein, glycopeptide, lipopolysaccharide, glycolipid, petidoglycan, proteoglycan, lipid, metalloprotein, liposome, or carbohydrate.

In an embodiment, at least one agent modulates the activity of MyD88. In an embodiment, at least one agent inhibits the activity of MyD88. In an embodiment, at least one agent inhibits the activity of one or more Toll-like receptors. In an embodiment, the Toll-like receptors include but are not limited to Toll-like receptor 1, Toll-like receptor 2, Toll-like receptor 3, Toll-like receptor 4, Toll-like receptor 5, Toll-like receptor 6, Toll-like receptor 7, Toll-like receptor 8, Toll-like receptor 9, Toll-like receptor 10, Toll-like receptor 11, Toll-like receptor 12, Toll-like receptor 13, or Toll-like receptor 14. In an embodiment, at least one agent includes at least one of M62812, chloroquine or quinine.

In an embodiment, at least one agent modulates the activity of one or more Src family kinases. In an embodiment, at least one agent inhibits the activity of one or more Src family kinases. In an embodiment, the Src family kinases include but are not limited to, Src, Lck, Hck, Fyn, Blk, Lyn, Fgr, Yes, or Yrk. In an embodiment, at least one agent includes at least one tyrosine kinase inhibitor including, but not limited to, at least one of a 2-aminothiazole, an aminoquinazoline, or an aminopyrimidine amide. In an embodiment, at least one agent includes, but is not limited to, one or more of dasatinib, nilotinib, BMS-268770, UR-12947, aztreonam, MZ-338, riluzole, meloxicam, pramipexole, CBS-113-A, AZD0530, bosutinib, INNO-406, MK-0457, cediranib, sunitinib, bosutinib, axitinib, erlotinib, gefitinib, lapatinib, lestaurtinib, semaxanib, or imatinib. In an embodiment, at least one agent includes, but is not limited to, dasatinib. In at least one embodiment, the therapeutic composition includes chloroquine or quinine and at least one of dasatinib, disulfiram, or bortezomib. In at least one embodiment, the therapeutic composition includes chloroquine and dasatinib. In at least one embodiment, the therapeutic composition includes quinine and dasatinib.

In an embodiment, a therapeutic composition is described herein that includes at least two agents, wherein at least one agent inhibits the activity of Toll-like receptor 9, and at least one agent inhibits the activity of Hck or Lyn.

In an embodiment, the therapeutic composition further includes at least one third agent, wherein the at least one third agent is configured to modulate the activity of at least one transcription factor. In an embodiment, the at least one third agent is configured to modulate the activity of at least one of NF-kB complex, NF-kB subunit, NF-kB co-activator, or histone deacetylase. In an embodiment, the at least one third agent inhibits the activity of at least one of NF-kB complex, NF-kB subunit, NF-kB co-activator, or histone deacetylase.

In an embodiment, the at least one third agent includes at least one biohydrolyzable carbarnate. In an embodiment, the at least one third agent includes at least one moiety capable of binding one or more metal ions including iron or copper. In an embodiment, the at least third agent includes one or more of disulfiram, ditiocarb, sulindac, sulfasalazine, or bortezomib.

In an embodiment, the therapeutic composition includes at least one fourth agent that modulates the activity of at least one protease or proteasome. In at least one embodiment, the at least one fourth agent inhibits the activity of at least one protease or at least one proteasome. In an embodiment, the at least one fourth agent includes dichloroisocoumarin, squinavir, ritonavir, indinavir, nelfinavir, amprenavir, lopinavir, atazanavir, fosamprenavir, tipranavir, darunavir, or Cathepsin K. In an embodiment, the at least one protease includes one or more cysteine proteases. In an embodiment, the at least one protease includes one or more serine proteases. In an embodiment, the at least one protease includes one or more of PfSUB1, PfSUB2, DPAP1, DPAP2, DPAP3. In an embodiment, the at least one protease inhibits the activity of one or more of SERA1, SERA2, SERA3, SERA4, SERA5, SERA6, SERA7, or SERA8. In an embodiment, the at least one proteasome includes 26S Proteasome.

In an embodiment, the therapeutic composition is configured to modulate the production of at least one cytokine. In an embodiment, the therapeutic composition inhibits the production of at least one cytokine. In an embodiment, the at least one cytokine includes one or more members of the α-helix bundle cytokine family. In an embodiment, the at least one cytokine includes one or more of IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, IL-39, IL-40, IL-41, IL-42, IFN-γ, IFN-α, IFN-β, or TNF-α.

In an embodiment, the at least one cytokine includes one or more chemokines. In an embodiment, the at least one chemokine includes, but is not limited to, at least one of a CC chemokine, CXC chemokine, C chemokine, or CX3C chemokine. In an embodiment, the one or more chemokines includes, but is not limited to, CCL1, CCL2, CCL3, CCL4, CCL5, CCL6, CCL7, CCL8, CCL9/CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CCL29, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, CXCL18, CXCL19, CXCL20, CXCL21, CXCL22, XCL1, XCL2, XCL3, XCL4, XCL5, CX3CL1, CX3CL2, or CX3CL3.

In an embodiment, the therapeutic composition further includes at least one of sulfadoxine-pyrimethamine, mefloquine, doxycyline, atovaquone-proguanil, artemether, arteether, artelinic acid, artemotil, dikydroartemisin, dihydroartemisin-piperaquine, amodiaquine, lumefantrine, artesunate, artemisinin, or primaquine.

In an embodiment, the therapeutic composition includes at least one pharmaceutically-acceptable carrier or excipient. In an embodiment, the therapeutic composition includes a time-release formulation. In an embodiment, the therapeutic composition includes at least one solid, liquid or gas. In an embodiment, the therapeutic composition includes at least one of an aerosol, gel, sol, ointment, solution, suspension, capsule, tablet, suppository, cream, device, paste, liniment, lotion, ampule, elixir, emulsion, microemulsion, spray, suspension, powder, syrup, tincture, detection material, polymer, biopolymer, buffer, adjuvant, diluent, lubricant, disintegration agent, suspending agent, solvent, colorant, glidant, anti-adherent, anti-static agent, surfactant, plasticizer, emulsifying agent, flavor, gum, sweetener, coating, binder, filler, compression aid, encapsulation aid, preservative, granulation agent, spheronization agent, stabilizer, adhesive, pigment, sorbent, or nanoparticle. In an embodiment, the therapeutic composition is formulated for delivery to a subject by at least one of peroral delivery, oral delivery, topical delivery, transdermal delivery, epidermal delivery, intravitreal delivery, transmucosal delivery, inhalation, surgical delivery, or injection delivery.

In an embodiment, the therapeutic composition includes at least one of M62812, chloroquine or quinine; and at least one of dasatinib, nilotinib, BMS-268770, UR-12947, aztreonam, MZ-338, riluzole, meloxicam, pramipexole, CBS-113-A, AZD0530, bosutinib, INNO-406, MK-0457, cediranib, sunitinib, bosutinib, axitinib, erlotinib, gefitinib, lapatinib, lestaurtinib, semaxanib, or imatinib. In this or another embodiment, the therapeutic composition includes at least one pharmaceutically-acceptable carrier or excipient.

In an embodiment, the therapeutic composition includes at least one of M62812, chloroquine or quinine; and at least one of disulfiram, ditiocarb, sulindac, sulfasalazine, or bortezomib.

In an embodiment, the therapeutic composition includes at least one of disulfiram, ditiocarb, sulindac, sulfasalazine, or bortezomib; and at least one of dasatinib, nilotinib, BMS-268770, UR-12947, aztreonam, MZ-338, riluzole, meloxicam, pramipexole, CBS-113-A, cediranib, sunitinib, bosutinib, axitinib, erlotinib, gefitinib, lapatinib, lestaurtinib, semaxanib or imatinib.

In an embodiment, the therapeutic composition includes chloroquine or quinine; and disulfiram. In at least one embodiment, the therapeutic composition includes at least one pharmaceutically-acceptable carrier or excipient.

One aspect relates to methods including, but not limited to, modulating cellular activities. In an embodiment, the amount of one or more therapeutic agents or therapeutic compositions described herein and utilized in a method described herein are selected based on one or more attributes of the subject. In an embodiment, the one or more attributes of the subject include phenotypic or genotypic attributes. In an embodiment, the one or more attributes of the subject include one or more of a physiological condition, genetic or proteomic profile, genetic or proteomic characteristic, response to previous treatment, weight, height, medical diagnosis, familial background, results of one or more medical tests, ethnic background, body mass index, age, presence or absence of at least one disease or condition, species, ethnicity, race, allergies, gender, presense or absence of at least one biological, chemical, or therapeutic agent in the subject, pregnancy status, lactation status, medical history, or blood condition.

In an embodiment, the method includes modulating at least one immune response of one or more cells of a subject by administering to the subject an effective amount of at least one therapeutic composition described herein.

In an embodiment, the one or more cells are located at least one of in vitro, in vivo, in situ, in utero, or ex vivo. In an embodiment, the one or more cells are located in a subject that is afflicted with or suspected of being afflicted with at least one inflammatory disease or condition. In an embodiment, the at least one inflammatory disease or condition includes, but is not limited to, one or more of a pathogenic infection, parasitic infection, autoimmune disease, allergic reaction, or cancer.

In an embodiment, the parasitic infection includes, but is not limited to, at least one infection or infestation of one or more of a phytoparasite, zooparasite, ectoparasite, endoparasite, or one or more of parasitic cysts, larvae, or eggs. In an embodiment, the at least one inflammatory disease or condition includes, but is not limited to, one or more of anaphylaxis, viral infection, bacterial infection, plasmodium infection, protozoan infection, nematode infection, or worm infection. In an embodiment, the at least one inflammatory disease or condition includes malaria.

In an embodiment, the method further includes, but is not limited to, detecting in the subject at least one level of at least one biological signaling molecule that is associated with at least one inflammatory disease or condition. In an embodiment, the method further includes, but is not limited to, analyzing one or more biological tissues or fluids from the subject. In an embodiment, the one or more biological tissues or fluids from the subject are analyzed by utilizing one or more of thin-layer chromatography, mass spectrometry, nuclear magnetic resonance, polymerase chain reaction, reverse transcriptase, Northern blot, Western blot, microscopy, flow cytometry, antibody binding, enzyme-linked immunosorbent assay, radioactive absorption or release, cell counting, or cell sorting.

In an embodiment, the at least one biological signaling molecule includes, but is not limited to, one or more of a nucleic acid, amino acid, peptide, polypeptide, protein, carbohydrate, lipid, glycoprotein, glycopeptide, lipopolysaccharide, glycolipid, metalloprotein, or proteoglycan. In an embodiment, the at least one biological signaling molecule includes, but is not limited to, one or more of a cytokine, chemokine, cellular receptor, intracellular second messenger, protease, kinase, enzyme, cellular receptor ligand, transcription factor, or hormone.

In an embodiment, the subject includes, but is not limited to, at least one vertebrate or invertebrate. In an embodiment, the subject includes, but is not limited to, at least one of a fish, reptile, mammal, amphibian, or bird. In an embodiment, the subject includes, but is not limited to, at least one human. In at least one embodiment, the method of treatment is based on a genetic or proteomic profile of the subject. In at least one embodiment, the method of treatment is based on one or more polymorphisms. The one or more polymorphisms can be confirmed or presumed at the time of treatment.

An embodiment includes a method of modulating at least one immune response of one or more cells of a subject, comprising: administering to the subject an effective amount of at least one therapeutic composition, including chloroquine or quinine; dasatinib; and at least one pharmaceutically-acceptable carrier or excipient. In at least one embodiment, the method of modulating at least one immune response of one or more cells of a subject includes administering to the subject an effective amount of at least one therapeutic composition, including chloroquine or quinine; dasatinib; bortezomib; and at least one pharmaceutically-acceptable carrier or excipient.

An embodiment relates to modulating the activity of one or more Toll-like receptors and one or more Src family kinases in one or more cells of a subject by administering to the subject an effective amount of at least one therapeutic composition described herein. An embodiment relates to modulating the activity of one or more Toll-like receptors and one or more NF-kB molecules or other transcription factors in one or more cells of a subject by administering to the subject an effective amount of at least one therapeutic composition described herein.

An embodiment relates to modulating the activity of one or more NF-kB molecules or other transcription factors and one or more Src family kinases in one or more cells of a subject by administering to the subject an effective amount of at least one therapeutic composition described herein.

An embodiment relates to modulating the activity of one or more Toll-like receptors, one or more Src family kinases, and one or more NF-kB molecules or other transcription factors in one or more cells of a subject by administering to the subject an effective amount of at least one therapeutic composition described herein.

In an embodiment, the one or more cells are located at least one of in vitro, in vivo, in situ, in utero, or ex vivo. In an embodiment, the one or more cells are located in a subject that is afflicted with or suspected of being afflicted with at least one inflammatory disease or condition. In an embodiment, the at least one inflammatory disease or condition includes, but is not limited to, one or more of a pathogenic infection, parasitic infection, autoimmune disease, allergic reaction, or cancer.

In an embodiment, the parasitic infection includes, but is not limited to, at least one infection or infestation of one or more of a phytoparasite, zooparasite, ectoparasite, endoparasite, or one or more of parasitic cysts, larvae, or eggs. In an embodiment, the at least one inflammatory disease or condition includes, but is not limited to, one or more of anaphylaxis, viral infection, bacterial infection, plasmodium infection, protozoan infection, nematode infection, or worm infection. In an embodiment, the at least one inflammatory disease or condition includes malaria.

In an embodiment, a method of treating a subject afflicted with or suspected of being afflicted with at least one inflammatory disease or condition, includes administering to a subject an effective amount of at least one therapeutic composition, including at least one of chloroquine, M62812, or quinine; at least one of disulfiram, ditiocarb, sulindac, salfasalazine, or bortezomib; and at least one pharmaceutically-acceptable carrier or excipient.

In an embodiment, a method of treating a subject afflicted with or suspected of being afflicted with malaria, includes administering to a subject an effective amount of at least one therapeutic composition, including at least one of chloroquine, M62812, or quinine; at least one of disulfiram, ditiocarb, sulindac, salfasalazine, or bortezomib; and at least one pharmaceutically-acceptable carrier or excipient.

In an embodiment, the method further includes, but is not limited to, detecting in the subject at least one level of at least one biological signaling molecule that is associated with at least one inflammatory disease or condition. In an embodiment, the method further includes, but is not limited to, analyzing one or more biological tissues or fluids from the subject. In an embodiment, the one or more biological tissues or fluids from the subject are analyzed by utilizing one or more of thin-layer chromatography, mass spectrometry, nuclear magnetic resonance, polymerase chain reaction, reverse transcriptase, Northern blot, Western blot, microscopy, flow cytometry, antibody binding, enzyme-linked immunosorbent assay, radioactive absorption or release, cell counting, or cell sorting.

In an embodiment, the at least one biological signaling molecule includes, but is not limited to, one or more of a nucleic acid, amino acid, peptide, polypeptide, protein, carbohydrate, lipid, glycoprotein, glycopeptide, glycolipid, metalloprotein, or proteoglycan. In an embodiment, the at least one biological signaling molecule includes, but is not limited to, one or more of a cytokine, chemokine, cellular receptor, intracellular second messenger, protease, kinase, enzyme, cellular receptor ligand, transcription factor, or hormone. In at least one embodiment, the at least one therapeutic composition includes a time-release formulation. An embodiment includes a method of modulating the activity of one or more Toll-like receptors and one or more Src family kinases in one or more cells of a subject, including administering to the subject an effective amount of at least one therapeutic composition, including at least one of chloroquine or quinine, dasatnib; and at least one pharmaceutically-acceptable carrier or excipient.

In an embodiment, the subject includes, but is not limited to, at least one vertebrate or invertebrate. In an embodiment, the subject includes, but is not limited to, at least one of a fish, reptile, mammal, amphibian, or bird. In an embodiment, the subject includes, but is not limited to, at least one human.

In an embodiment, the method includes, but is not limited to, treating a subject afflicted with at least one inflammatory disease or condition by administering to the subject an effective amount of at least one therapeutic composition including at least one of chloroquine or quinine; and at least one of dasatinib, nilotinib, BMS-268770, UR-12947, aztreonam, MZ-338, riluzole, meloxicam, pramipexole, CBS-113-A, cediranib, sunitinib, bosutinib, axitinib, erlotinib, gefitinib, lapatinib, lestaurtinib, semaxanib or imatinib. In an embodiment, the at least one therapeutic composition includes Cathepsin K or dichloroisocoumarin. In an embodiment, the at least one therapeutic composition includes at least one of sulfadoxine-pyrimethamine, mefloquine, doxycycline, atovaquone-proguanil, artemether, arteether, artelinic acid, artemotil, dihydroartemisin, dihydroartemisin-piperaquine, amodiaquine, lumefantrine, artesunate, artemisinin, or primaquine. In an embodiment, the at least one therapeutic composition may include at least one pharmaceutically-acceptable carrier or excipient.

In an embodiment, the method includes, but is not limited to, treating a subject afflicted with at least one inflammatory disease or condition by administering to the subject an effective amount of at least one therapeutic composition including at least one of chloroquine or quinine and at least one of disulfiram, ditiocarb, or bortezomib. In an embodiment, the at least one therapeutic composition includes Cathepsin K or dichloroisocoumarin. In an embodiment, the at least one therapeutic composition includes at least one of sulfadoxine-pyrimethamine, mefloquine, doxycycline, atovaquone-proguanil, artemether, arteether, artelinic acid, artemotil, dihydroartemisin, dihydroartemisin-piperaquine, amodiaquine, lumefantrine, artesunate, artemisinin, or primaquine. In an embodiment, the at least one therapeutic composition may include at least one pharmaceutically-acceptable carrier or excipient. In an embodiment, the method includes treating a subject afflicted with or suspected of being afflicted with at least one inflammatory disease or condition, including administering to the subject an effective amount of at least one therapeutic composition, including chloroquine; dasatinib; and at least one pharmaceutically-acceptable carrier or excipient.

In an embodiment, the method includes, but is not limited to, treating a subject afflicted with at least one inflammatory disease or condition by administering to the subject an effective amount of at least one therapeutic composition including at least one of dasatinib, nilotinib, BMS-268770, UR-12947, aztreonam, MZ-338, riluzole, meloxicam, pramipexole, CBS-113-A, cediranib, sunitinib, bosutinib, axitinib, erlotinib, gefitinib, lapatinib, lestaurtinib, semaxanib or imatinib; and at least one of disulfiram, ditiocarb, or bortezomib. In an embodiment, the at least one therapeutic composition includes Cathepsin K or dichloroisocoumarin. In an embodiment, the at least one therapeutic composition includes at least one of sulfadoxine-pyrimethamine, mefloquine, doxycycline, atovaquone-proguanil, artemether, arteether, artelinic acid, artemotil, dihydroartemisin, dihydroartemisin-piperaquine, amodiaquine, lumefantrine, artesunate, artemisinin, or primaquine. In an embodiment, the at least one therapeutic composition may include at least one pharmaceutically-acceptable carrier or excipient.

In an embodiment, the method includes, but is not limited to, treating a subject afflicted with at least one inflammatory disease or condition by administering to the subject an effective amount of at least one therapeutic composition including at least one of chloroquine or quinine; at least one of dasatinib, nilotinib, BMS-268770, UR-12947, aztreonam, MZ-338, riluzole, meloxicam, pramipexole, CBS-113-A, cediranib, sunitinib, bosutinib, axitinib, erlotinib, gefitinib, lapatinib, lestaurtinib, semaxanib or imatinib; and at least one of disulfiram, ditiocarb, or bortezomib. In an embodiment, the at least one therapeutic composition includes Cathepsin K or dichloroisocoumarin. In an embodiment, the at least one therapeutic composition includes at least one of sulfadoxine-pyrimethamine, mefloquine, doxycycline, atovaquone-proguanil, artemether, arteether, artelinic acid, artemotil, dihydroartemisin, dihydroartemisin-piperaquine, amodiaquine, lumefantrine, artesunate, artemisinin, or primaquine. In an embodiment, the at least one therapeutic composition may include at least one pharmaceutically-acceptable carrier or excipient.

In an embodiment, the method includes, but is not limited to, treating a subject afflicted with or suspected of being afflicted with malaria by administering to the subject an effective amount of at least one therapeutic composition including at least one of chloroquine or quinine; and at least one of dasatinib, nilotinib, BMS-268770, UR-12947, aztreonam, MZ-338, riluzole, meloxicam, pramipexole, CBS-113-A, cediranib, sunitinib, bosutinib, axitinib, erlotinib, gefitinib, lapatinib, lestaurtinib, semaxanib or imatinib. In an embodiment, the at least one therapeutic composition includes Cathepsin K or dichloroisocoumarin. In an embodiment, the at least one therapeutic composition includes at least one of sulfadoxine-pyrimethamine, mefloquine, doxycycline, atovaquone-proguanil, artemether, arteether, artelinic acid, artemotil, dihydroartemisin, dihydroartemisin-piperaquine, amodiaquine, lumefantrine, artesunate, artemisinin, or primaquine. In an embodiment, the at least one therapeutic composition may include at least one pharmaceutically-acceptable carrier or excipient.

In an embodiment, the method includes, but is not limited to, treating a subject afflicted with or suspected of being afflicted with malaria by administering to the subject an effective amount of at least one therapeutic composition including at least one of chloroquine or quinine; and at least one of disulfiram, ditiocarb, or bortezomib. In an embodiment, the at least one therapeutic composition includes Cathepsin K or dichloroisocoumarin. In an embodiment, the at least one therapeutic composition includes at least one of sulfadoxine-pyrimethamine, mefloquine, doxycycline, atovaquone-proguanil, artemether, arteether, artelinic acid, artemotil, dihydroartemisin, dihydroartemisin-piperaquine, amodiaquine, lumefantrine, artesunate, artemisinin, or primaquine. In an embodiment, the at least one therapeutic composition may include at least one pharmaceutically-acceptable carrier or excipient.

In an embodiment, the method includes, but is not limited to, treating a subject afflicted with or suspected of being afflicted with malaria by administering to the subject an effective amount of at least one therapeutic composition including at least one of disulfiram, ditiocarb, or bortezomib; and at least one of dasatinib, nilotinib, BMS-268770, UR-12947, aztreonam, MZ-338, riluzole, meloxicam, pramipexole, CBS-113-A, cediranib, sunitinib, bosutinib, axitinib, erlotinib, gefitinib, lapatinib, lestaurtinib, semaxanib or imatinib. In an embodiment, the at least one therapeutic composition includes Cathepsin K or dichloroisocoumarin. In an embodiment, the at least one therapeutic composition includes at least one of sulfadoxine-pyrimethamine, mefloquine, doxycycline, atovaquone-proguanil, artemether, arteether, artelinic acid, artemotil, dihydroartemisin, dihydroartemisin-piperaquine, amodiaquine, lumefantrine, artesunate, artemisinin, or primaquine. In an embodiment, the at least one therapeutic composition may include at least one pharmaceutically-acceptable carrier or excipient.

In an embodiment, the method includes, but is not limited to, treating a subject afflicted with or suspected of being afflicted with malaria by administering to the subject an effective amount of at least one therapeutic composition including at least one of disulfiram, ditiocarb, or bortezomib; at least one of dasatinib, nilotinib, BMS-268770, UR-12947, aztreonam, MZ-338, riluzole, meloxicam, pramipexole, CBS-113-A, cediranib, sunitinib, bosutinib, axitinib, erlotinib, gefitinib, lapatinib, lestaurtinib, semaxanib or imatinib; and at least one of chloroquine or quinine. In an embodiment, the at least one therapeutic composition includes Cathepsin K or dichloroisocoumarin. In an embodiment, the at least one therapeutic composition includes at least one of sulfadoxine-pyrimethamine, mefloquine, doxycycline, atovaquone-proguanil, artemether, arteether, artelinic acid, artemotil, dihydroartemisin, dihydroartemisin-piperaquine, amodiaquine, lumefantrine, artesunate, artemisinin, or primaquine. In an embodiment, the at least one therapeutic composition may include at least one pharmaceutically-acceptable carrier or excipient.

In one aspect, the therapeutic compositions described herein may be administered to a subject by any delivery mechanism. Devices may be external, implantable, or implanted. An implanted drug delivery device includes, but is not limited to, at least one reservoir configured to receive, retain and dispense at least one therapeutic composition described herein. In an embodiment, the device is implantable. In an embodiment, the device is implanted into a subject. In an embodiment, the device is external to the subject.

In an embodiment, the device includes one or more controllable output mechanisms operably linked to the one or more outlets to control the dispensing of at least a portion of the at least one therapeutic composition from the at least one reservoir. In an embodiment, the at least one controllable output mechanism includes a micropump. In an embodiment, the at least one controllable output mechanism includes at least one thermal or nonthermal gate in communication with the at least one outlet of the at least one reservoir. In an embodiment, the device includes at least one control circuitry configured to control the at least one controllable output mechanism. In an embodiment, the at least one control circuitry is configured to generate and transmit an electromagnetic control signal configured to control the at least one controllable output mechanism.

In an embodiment, the device includes a memory mechanism for storing instructions for generating and transmitting the electromagnetic control signal. In an embodiment, the device includes at least one sensor for detecting the presence or level of one or more biological signaling molecules. In an embodiment, the at least one sensor for detecting the presence or level of one or more biological signaling molecules includes one or more recognition molecules specific to the one or more biological signaling molecules. In an embodiment, the biological signaling molecules include one or more detection indicators including, but not limited to, at least one dye, radioactive label, fluorescent label, electromagnetic label, magnetic label, or other detectable label.

In an embodiment, the one or more biological signaling molecules include at least one of a nucleic acid, amino acid, peptide, polypeptide, protein, glycopeptide, glycoprotein, glycolipid, peptidoglycan, proteoglycan, lipid, metalloprotein, liposome, or carbohydrate. In an embodiment, the one or more biological signaling molecules include at least one of a cytokine, intercellular messenger, intracellular messenger, neurotransmitter, hormone, signal transduction messenger, antibody or fragment thereof, or enzyme.

In an embodiment, the device includes an imaging apparatus capable of imaging the levels of the one or more biological signaling molecules within a therapeutically effective region. In an embodiment, the device includes an imaging apparatus capable of imaging the levels of the at least one therapeutic composition within a therapeutically effective region.

In an embodiment, the device includes at least one sensor configured to detect at least one quantity of the at least one therapeutic composition in the at least one reservoir. In an embodiment, the device includes one or more detection indicators. In an embodiment, the one or more detection indicators include at least one dye, radioactive label, fluorescent label, electromagnetic label, magnetic label, or other detectable label.

In an embodiment, the at least one sensor configured to detect at least one quantity of the therapeutic composition in the at least one reservoir can be the same or same type of sensor as the at least one sensor for detecting the presence or level of one or more biological signaling molecules. In an embodiment, the at least one sensor is associated with the device. In an embodiment, the at least one sensor is configured to be located remotely from the device.

In an embodiment, the at least one reservoir includes one or more inlet mechanisms for receiving external delivery of the at least one therapeutic composition. In an embodiment, the device includes at least one memory location for recording information. In an embodiment, the at least one memory location is configured to record information regarding the at least one sensor or remote controller. In an embodiment, the at least one memory location is configured to record information regarding at least one of a sensed condition, history, or performance of the device. In an embodiment, the at least one memory location is configured to record information regarding at least one of the date, time, quantity of material delivered, presence of one or more biological signaling molecules, or level of one or more biological signaling molecules.

In an embodiment, the device includes an information transmission mechanism configured to transmit information recorded by the at least one electronic memory location. In an embodiment the at least one reservoir includes a flow regulator. In an embodiment, the device further comprises a time-release regulator for the release of the at least one therapeutic composition over time. In an embodiment, the device further includes a receiver configured to obtain release instructions or authorization to release the at least one therapeutic composition.

In an embodiment, two or more of the at least one first agent, the at least one second agent, the at least one third agent, or the at least one fourth agent reside in separate reservoirs. In an embodiment, two or more of the at least one first agent, the at least one second agent, the at least one third agent, or the at least one fourth agent are released separately. In an embodiment, two or more of the at least one first agent, the at least one second agent, the at least one third agent, or the at least one fourth agent are released approximately simultaneously.

In one aspect, the system includes, but is not limited to, a computer device; and instructions that when executed on the computing device cause the computing device to regulate dispensing of at least one drug delivery device device configured to retain and dispense at least one therapeutic composition to at least one subject, wherein the at least one therapeutic composition includes a therapeutic composition described herein. In an embodiment, the therapeutic composition further includes at least one pharmaceutically-acceptable carrier or excipient. In an embodiment, the amount of one or more of the at least one first agent, the at least one second agent, the at least one third agent, or the at least one fourth agent are selected based on one or more attributes of the subject. In an embodiment, the amount includes relative amount, absolute amount, or approximate amount. In an embodiment, the attributes of the subject include phenotypic or genotypic attributes. In an embodiment, the one or more attributes of the subject include one or more of a physiological condition, genetic or proteomic profile, genetic or proteomic characteristic, response to previous treatment, weight, height, medical diagnosis, familial background, results of one or more medical tests, ethnic background, body mass index, age, presence or absence of at least one disease or condition, species, ethnicity, race, allergies, gender, presence or absence of at least one biological, chemical, or therapeutic agent in the subject, pregnancy status, lactation status, medical history, or blood condition.

In an embodiment, the system includes, but is not limited to, a computing device including a personal digital assistant (PDA), a laptop computer, a tablet personal computer, a networked computer, a computing system including a cluster of processors, a computing system including a cluster of servers, a mobile telephone, a workstation computer, or a desktop computer.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 illustrates an example of a signal transduction pathway related to inflammation.

FIG. 2 illustrates an example of a therapeutic composition delivery device.

FIG. 3 illustrates alternate embodiments of FIG. 2.

FIG. 4 illustrates alternate embodiments of FIG. 2.

FIG. 5 illustrates a partial view of a system 500 that includes a computer program for executing a computing process on a computing device.

FIG. 6 illustrates alternate embodiments of FIG. 5.

FIG. 7 illustrates a partial view of a system 600 that includes a computer program for executing a computing process on a computing device.

FIG. 8 illustrates alternate embodiments of FIG. 7.

FIG. 9 illustrates a partial view of a system 700 that includes a computer program for executing a computing process on a computing device.

FIG. 10 illustrates alternate embodiments of FIG. 9.

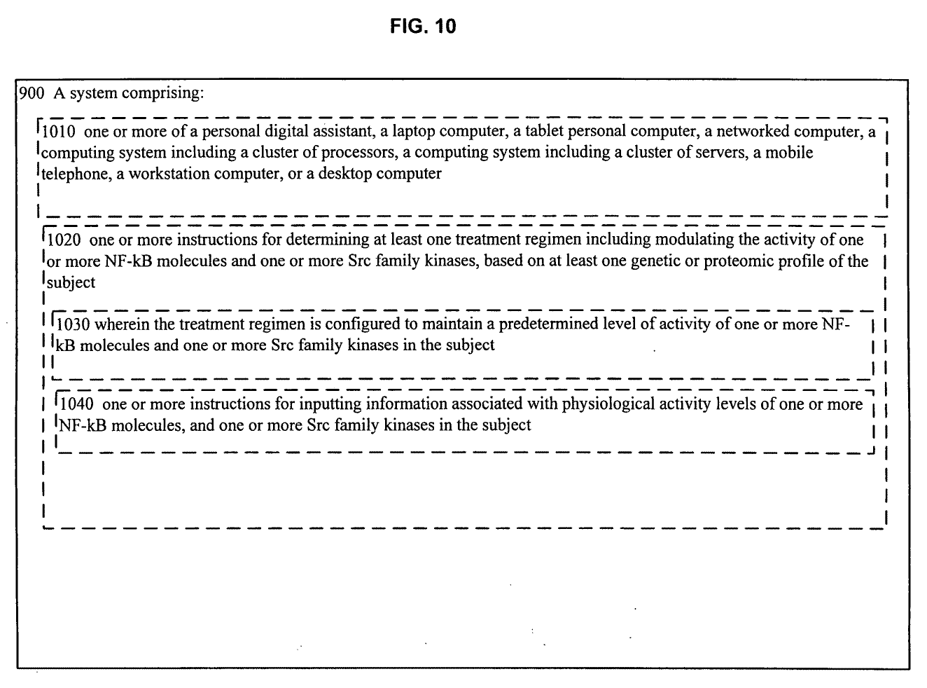

FIG. 11 illustrates a partial view of a system 800 that includes a computer program for executing a computing process on a computing device.

FIG. 12 illustrates alternate embodiments of FIG. 11.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present application uses formal outline headings for clarity of presentation. However, it is to be understood that the outline headings are for presentation purposes, and that different types of subject matter may be discussed throughout the application (e.g., method(s) may be described under composition heading(s) and/or kit headings; and/or descriptions of single topics may span two or more topic headings). Hence, the use of the formal outline headings is not intended to be in any way limiting.

The therapeutic compositions, methods, devices, and systems described herein relate to multiple agents that modulate inflammatory reactions. General inflammatory reactions produce signs or symptoms in the subject that include, but are not limited to, shivering, sensation of cold, fever, heat from a specific area of the subject's body, muscle pain, aches, redness, loss of function, headaches, sweating, malaise, loss of appetite, sleepiness, increased blood pressure, nausea and vomiting, pain, mild jaundice, enlarged liver, enlarged spleen, enlarged joints, swelling, and possibly seizures. Modulating inflammatory reactions can reduce or eliminate some or all of these signs or symptoms.

Intracellular signaling pathways contribute to biochemical cascades that result in multiple events. In certain circumstances, inflammation is one of these events. In certain embodiments described herein, the activity of at least two signaling molecules is modulated. In an embodiment, a therapeutic composition includes at least one first agent configured to modulate the activity of one or more Toll-like receptors (TLR), at least one second agent configured to modulate the activity of one or more Src family kinases; and at least one pharmaceutically-acceptable carrier or excipient.

In an embodiment, a therapeutic composition includes at least one first agent configured to modulate the activity of one or more Toll-like receptors; at least one second agent configured to modulate the activity of one or more NF-kB molecules; and at least one pharmaceutically-acceptable carrier or excipient. In an embodiment, a therapeutic composition includes at least one first agent configured to modulate the activity of one or more NF-kB molecules; at least one second agent configured to modulate the activity of one or more Src family kinases; and at least one pharmaceutically-acceptable carrier or excipient. In an embodiment, a therapeutic composition includes at least one first agent configured to modulate the activity of one or more Toll-like receptors; at least one second agent configured to modulate the activity of one or more Src family kinases; at least one third agent configured to modulate the activity of one or more NF-kB molecules; and at least one pharmaceutically-acceptable carrier or excipient.

In at least one embodiment, one or more of the at least one first agent, at least one second agent, or at least one third agent includes one or more of an organic or inorganic small molecule, nucleic acid, amino acid, peptide, polypeptide, protein, glycoprotein, glycopeptide, glycolipid, lipopolysaccharide, peptidoglycan, proteoglycan, lipid, metalloprotein, liposome, or carbohydrate.

In at least one embodiment, the at least one agent configured to modulate the activity of one or more Toll-like receptors also modulates the activity of MyD88. In at least one embodiment, the at least one agent inhibits the activity of MyD88. In at least one embodiment, the at least one agent inhibits the activity of one or more Toll-like receptors.

The Toll and Toll-like receptor family are type I transmembrane proteins that have been isolated in both vertebrate and invertebrate species. In humans, the Toll-like receptors are expressed on cells of the immune system, and operate as a first line of defense against microorganisms, including bacteria, viruses, protozoa, and fungi. Without wishing to be bound by any particular theory, it is believed that activation of most of the TLRs leads to translocation of NF-kB to the cell nucleus, and release of proinflammatory cytokines. (See e.g., Schumann, PNAS, Vol. 104, No. 6, pp. 1743-1744 (2007), which is herein incorporated by reference).

At least fourteen Toll-like receptors have been identified, Toll-like receptor 1, Toll-like receptor 2, Toll-like receptor 3, Toll-like receptor 4, Toll-like receptor 5, Toll-like receptor 6, Toll-like receptor 7, Toll-like receptor 8, Toll-like receptor 9, Toll-like receptor 10, Toll-like receptor 11, Toll-like receptor 12, Toll-like receptor 13, and Toll-like receptor 14. In at least one embodiment, one or more therapeutic compositions described herein modulate one or more of these Toll-like receptors, and in at least one embodiment, one or more therapeutic compositions described herein modulate the activity of one or more Toll-like receptors. In an embodiment, the one or more therapeutic compositions described herein inhibit the activity of one or more Toll-like receptors. In at least one embodiment, the at least one first agent includes at least one of chloroquine, quinine, or M62812.

Chloroquine, a 4-aminoquinoline therapeutic has been used in the treatment or prevention of malaria, and as an anti-retroviral agent. Chloroquine does not inhibit CpG-induced Src family kinase activation, or its dependent cellular responses. (See e.g., Sanjuan et al., J. Cell Biol., Vol. 172, No. 7, pp. 1057-1068 (2006), which is herein incorporated by reference).

Quinine is a stereoisomer of quinidine, and has been used widely as an antimalarial drug. M62812, or 3-amino-6-(2-aminophenoxy)-1,2-benzisothiazole dihydrochloride, is an inhibitor of Toll-like receptor 4 and prevents lethal septic shock in mice. (See e.g., Nakamura et al., Eur. J. Pharm., Vol. 569, No. 3, pp. 237-243 (2007), which is herein incorporated by reference).

MyD88 is an adapter protein that is involved in IL-1 and Toll-like receptor activation of NF-kB. Anti-sense oligonucleic acids specific for MyD88, as well as methods for modulating the expression of MyD88 have been described. (See e.g., U.S. application Ser. No. 11/339,785, Pub. No. 2006/0172962, which is herein incorporated by reference).

The Src family of tyrosine kinases was first found in a sarcoma virus, and is now known to be involved with many cellular processes. Exemplary members of the Src family of tyrosine kinases include, but are not limited to, c-Src, v-Src, Frk, Fgr, Blk, Syk, Yes, Lyn, Hck, Fyn, and Lck. In at least one embodiment, the at least one agent configured to modulate the activity of at one or more Src family kinases, modulates the activity of c-Src, v-Src, Frk, Fgr, Blk, Syk, Yes, Lyn, Hck, Fyn, or Lck.

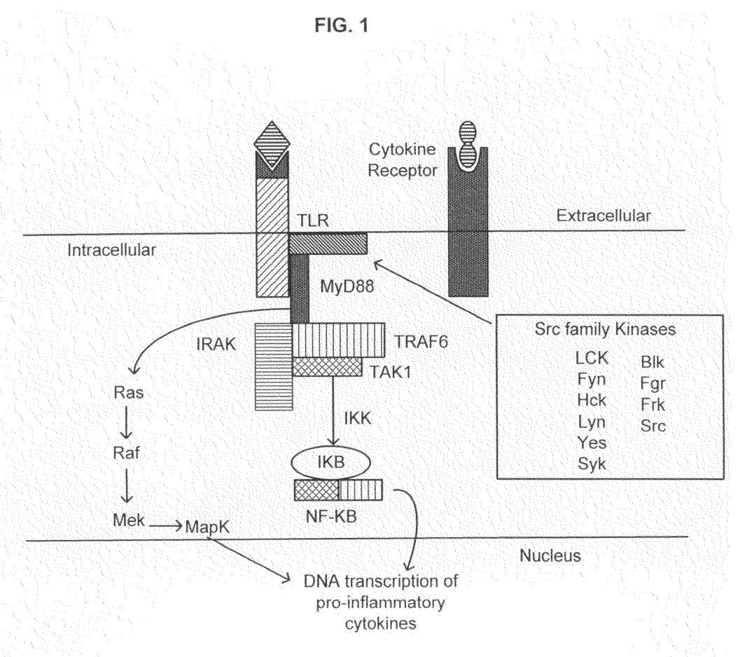

As illustrated in FIG. 1, Toll-like receptor-ligand interaction results in at least one downstream signaling cascade that includes one or more of MyD88, TRAF6, TAK 1, IKK, IKB, NF-kB, IRAK, Ras, Raf, Mek, MapK (and other Map kinases), Src family kinases, and can result in DNA transcription of, for example, cytokine (e.g., pro-inflammatory cytokines). In at least one embodiment described herein, at least one therapeutic composition modulates at least two points in the pathway indicated in FIG. 1. This modulation may include, for example, inhibition, interruption of signaling, or increasing or decreasing activity of a particular signaling molecule or receptor.

In at least one embodiment, the at least one agent configured to modulate the activity of one or more Src family kinases inhibits one or more of these members. In at least one embodiment, the at least one agent configured to modulate the activity of one or more Src family kinases includes one or more of a 2-aminothiazole, an aminoquinazoline, or an aminopyrimidine amide. In at least one embodiment, the at least one agent configured to modulate the activity of one or more Src family kinases includes one or more of dasatinib, nilotinib, BMS-268770, UR-12947, aztreonam, MZ-338, riluzole, meloxicam, pramipexole, CBS-113-A, AZD0530, bosutinib, INNO-406, MK-0457, cediranib, sunitinib, bosutinib, axitinib, erlotinib, gefitinib, lapatinib, lestaurtinib, semaxanib or imatinib. At least one member of the Src family of kinases is activated by microbial infection, such as viral infection, and associates with one or more Toll-like receptor. (See e.g., Johnsen, et al., EMBO J., Vol. 25, No. 14, pp. 3335-3346 (2006), which is herein incorporated by reference).

Dasatinib (SPRYCEL™) is a drug approved by the U.S. Food and Drug Administration for the treatment of adults with chronic, accelerated, or myeloid or lymphoid blast phase chronic myeloid leukemia with resistance or intolerance to prior therapy, including imatinib; and for the treatment of adults with Philadelphia chromosome-positive acute lymphoblastic leukemia with resistance or intolerance to prior therapy. At nanomolar concentrations, dasatinib inhibits BCR-ABL, Src family kinases (Src, Lck, Yes, Fyn), c-Kit, Ephal, and PDGFRβ. (See e.g., Product information, www.fda.gov/cder/foi/label/2006/0219861b1.pdf, which is herein incorporated by reference). Nilotinib, cediranib, sunitinib, bosutinib, axitinib, erlotinib, gefitinib, lapatinib, lestaurtinib, semaxanib, and imatinib are tyrosine kinase inhibitors, while BMS-268770 is a CDK2 inhibitor and UR-12947 is a fibrinogen receptor agonist.

In at least one embodiment, a therapeutic composition includes at least one agent configured to modulate the activity of Toll-like receptor 9. In at least one embodiment, the agent inhibits the activity of Toll-like receptor 9. In at least one embodiment, a therapeutic composition includes at least one agent configured to modulate Hck or Lyn. In at least one embodiment, the therapeutic composition inhibits the activity of Hck or Lyn.

In at least one embodiment, a therapeutic composition includes at least one agent configured to modulate the activity of one or more transcription factors. In at least one embodiment, a therapeutic composition includes at least one agent configured to inhibit the activity of one or more transcription factors.

Transcription factors, such as NF-kB are involved with immune and inflammatory responses, whose activity is mediated through interactions with an inhibitor protein, IkB. Without wishing to be bound by any particular theory, NF-kB is maintained in an inactive form in the nucleus, and is activated by phosphorylation of IkB, which leads to degradation of IkB through the ubiquitin-proteasome pathway. 26S proteasome is particularly involved in degradation of cellular proteins, including ubiquitinated IkB. (See e.g., Cusack, et al., Cancer Res., Vol. 61, pp. 3535-3540 (2001), which is herein incorporated by reference). Inhibition of the proteasome maintains NF-kB in its inactive form. (See e.g., Cusack, et al., Cancer Res., pp. 3535-3540, Vol. 61, 2001, which is herein incorporated by reference). PS-341, a boronic acid dipeptide that is selective for proteasome inhibition, blocks activation of NF-kB in cancer cells. (See e.g., Cusack, et al., Cancer Res., Vol. 61, pp. 3535-3540 (2001), which is herein incorporated by reference). In at least one embodiment, the at least one agent configured to modulate the activity of one or more NF-kB molecules includes at least one moiety capable of binding one or more metal ions including iron or copper. In at least one embodiment, the at least one agent configured to modulate the activity of one or more NF-kB molecules includes at least one bihydrolyzable carbamate. In at least one embodiment, the agent configured to modulate the activity of one or more NF-kB molecules includes one or more of disulfiram, ditiocarb, sulindac, sulfasalazine, or bortezomib.

Dithiocarbamates and their complexes with metals are used as common pesticides, vulcanizing or analytical agents. Dithiocarbamates inhibit NF-kB activation, as well as proteasome degradation of IkB. (See e.g., Cvek and Dvorak, Curr. Pharm. Design, Vol. 13, pp. 1-13 (2007), which is herein incorporated by reference). The ubiquitin-proteasome system is useful for cellular maintenance of protein quality by degrading misfolded and denatured proteins. The proteasome also plays nonproteolytic roles in the cell, including but not limited to those involved in nucleic acid excision repair, recruitment of histone acetyltransferases to target promoters, transcription elongation, and cell cycle control. (See e.g., Cvek and Dvorak, Curr. Pharm. Design, Vol. 13, pp. 1-13 (2007), which is herein incorporated by reference).

Disulfiram is a member of the dithiocarbamate family of a molecules possessing an R₁R₂NC(S)SR₃ functional group, which is capable of forming metal complexes and reacting with sulfhydryl groups, wherein R₁ and R₂ at each occurrence are independently hydrogen, substituted or unsubstituted alkyl, cycloalkyl, heteroalkyl, alkoxy, alkenyl, alkynyl, aryl, heteroaryl, or heterocyclyl; M is a metal ion; each A is independently an anionic ligand; each B is independently a neutral ligand; each C is independently a cationic ligand; n is an integer from 1-10, where when n is greater than 1, each (S₂CNR₁R₂) may be the same or different; x, y and z are independently 0 or integers from 1-8; wherein the coordination number of M is an integer of 1-10; wherein the oxidation state of M is an integer of −1 to +8; wherein n, x, y and z are selected such that the coordination number and the oxidation state of the metal ion are satisfied; wherein the compound has an overall neutral charge; wherein each (S₂CNR₁R₂) portion of the compound is bound to the metal ion through one or both sulfur atoms; wherein each R₁ and R₂ may be the same or different; and wherein each A, B and C may be the same or different. (See e.g., Chen, et al., Cancer Res, Vol. 66, No. 21, pp. 10425-10433 (2006), and PCT Application No. WO 2006/023714, each of which is herein incorporated by reference). Disulfiram has the ability to bind copper, which in turn inhibits proteasomal activity in cultured breast cancer cells. (See e.g., Chen, et al., Cancer Res, Vol. 66, No. 21, pp. 10425-10433, (2006), which is herein incorporated by reference). Disulfiram inhibits aldehyde dehydrogenase without toxicity, and is approved by the U.S. Food and Drug Administration for treatment of alcoholism.

Diethyldithiocarbamate, a by-product of human metabolism of disulfiram, is a copper chelator, which has been shown to be toxic to malarial parasites, as well as other parasites including Leishmania, and Giardia. (See e.g., Meshnick et al., Biochem. Pharm. Vol. 40, No. 2, pp. 213-216, (1990); Nash et al., Antimicrobial Agents Chem. Vol. 42, No. 6, pp. 1488-1492 (1998), each of which is herein incorporated by reference).

In at least one embodiment, a therapeutic composition includes at least one agent configured to modulate the activity of at least one of NF-kB complex, NF-kB subunit, NF-kB co-activator, or histone deacetylase. In at least one embodiment, a therapeutic composition includes at least one agent configured to inhibit the activity of at least one of NF-kB complex, NF-kB subunit, NF-kB co-activator, or histone deaceytlase. In at least one embodiment, this agent is different than the agent configured to modulate the activity of one or more Toll-like receptors. In at least one embodiment this agent is different than the agent configured to modulate the activity of the one or more Src family kinases. In at least one embodiment, this agent is the same as the agent configured to modulate the activity of one or more Toll-like receptors. In at least one embodiment, this agent is the same as the agent configured to modulate the activity of the one or more Src family kinases.

Metals, such as iron, zinc, and copper, can affect the function of immune cells. (See e.g., Bonham, et al., Brit. J. Nutrition Vol. 87, pp.393-403, (2002), which is herein incorporated by reference). In particular, the effects of copper deficiency in a subject may result in at least one of the following: a decrease in microbicidal activities of neutrophils and peritoneal macrophages, a decrease in the number of antibody producing cells in spleens on exposure to erythrocytes from other species, a decrease in the cytolytic activity of natural killer cells, a decrease in delayed type hypersensitivity response, a decrease in in vitro responsiveness to T cell mitogens in splenic peripheral blood mononuclear cells, a decrease in the number of T lymphocytes, a decrease in T cell proliferation as measured by ³H thymidine incorporation into T cell DNA, a decrease in IL-2 levels, a decrease in superoxide dismutase activity, an increase in B cells, an increase in monocytes, and an increase in morbidity due to infection. (See e.g., Bonham, et al., Brit. J. Nutrition Vol. 87, pp. 393-403 (2002), which is herein incorporated by reference).

Inflammation related to infection or other causative agents may be mediated by proteases. In plasmodium infections, it has been shown that the subtilisin-family serine protease PfSUB1 and the cysteine protease dipeptidyl peptidase 3 (DPAP3) are regulators of the parasite's escape from host erythrocytes. (See e.g., Arastu-Kapur, et al., Nature Chem Biol, Vol. 4, No.3, pp. 203-213 (2008), which is herein incorporated by reference). Several proteins are processed during microorganism infection or rupture of cells in the infected subject. Some proteins that may play a role in parasitic infection include SERA 4, SERA5, and SERA6. (See e.g., Arastu-Kapur, et al., Nature Chem Biol, Vol. 4, No. 3, pp. 203-213 (2008), which is herein incorporated by reference).

In at least one embodiment, a therapeutic composition includes at least one fourth agent configured to modulate the activity of at least one protease or proteasome. In at least one embodiment, the at least one fourth agent inhibits the activity of at least one protease or proteasome. In at least one embodiment, the at least one fourth agent is the same as one or more of the at least one first agent, the at least one second agent, or the at least one third agent described herein. In at least one embodiment, the at least one fourth agent is different than one or more of the at least one first agent, the at least one second agent, or the at least one third agent described herein.

In at least one embodiment, one or more of the at least one first agent, at least one second agent, or at least one third agent includes one or more of an organic or inorganic small molecule, nucleic acid, amino acid, peptide, polypeptide, protein, glycoprotein, glycopeptide, glycolipid, lipopolysaccharide, peptidoglycan, proteoglycan, lipid, metalloprotein, liposome, or carbohydrate.

In at least one embodiment, the at least one protease includes one or more cysteine proteases. In at least one embodiment, the at least one protease includes one or more serine proteases. Inhibition of cathepsin K has been shown to reduce inflammation in autoimmune disease. (See e.g., Asagiri, et al., Science, Vol. 319, pp. 624-627 (2008), which is herein incorporated by reference). The cathepsins constitute a family of lysosomal cysteine proteases that were originally recognized as nonspecific scavengers of cellular proteins. Inhibition of cathepsin K results in defective Toll-like receptor 9 signaling in dendritic cells in response to unmethylated CpG DNA, which in turn leads to a number of events, including attenuated induction of T helper 17 cells. (See e.g., Asagiri, et al., Science, Vol. 319, pp. 624-627 (2008), which is herein incorporated by reference). In an embodiment, the at least one fourth agent inhibits Cathepsin K.

The protozoan Plasmodium parasites that cause malaria have a complex lifecycle that alternates between human- and mosquito-borne stages. An infective mosquito bite inoculates the subject with a sporozoite form of the protozoan that is briefly lodged in hepatocytes, and subsequent release of invasive merozoite forms that target erythrocytes. (See e.g., Lee et al., Nature Chem. Biol. Vol. 4, No. 3, pp. 161-162 (2008), which is herein incorporated by reference). Without wishing to be bound by any particular theory, it is believed that several proteases expressed by protozoa promote the release of the next generation of infective cells. In particular, PfSUB 1, as well as other subtilis in-like proteases, are involved in parasite egress from infected erythrocytes. (See e.g., Lee et al., Nature Chem. Biol. Vol. 4, No. 3, pp. 161-162 (2008), which is herein incorporated by reference).

In at least one embodiment, the at least one fourth agent inhibits at least one protease including PfSUB1, PfSUB2, DPAP1, DPAP2, or DPAP3. In at least one embodiment, the at least one protease modulates the activity of one or more of SERA1, SERA2, SERA3, SERA4, SERA5, SERA6, SERA7, or SERA8. In at least one embodiment, the at least one protease inhibits the activity of one or more of SERA1, SERA2, SERA3, SERA4, SERA5, SERA6, SERA7, or SERA8.

In at least one embodiment, the at least one agent configured to modulate the activity of at least one protease includes saquinavir, ritonavir, indinavir, nelfinavir, amprenavir, lopinavir, atazanavir, fosamprenavir, tipranavir, or darunavir.

Some exemplary proteasomes include, but are not limited to 26S proteasome, 20S proteasome, 19S proteasome, and the subunits thereof (e.g., S1, S2, S3, S4, S5, S6, S7, S8, S9, S10, S11, S12, S13, S14, or S15). In at least one embodiment, the at least one fourth agent inhibits the activity of 26S proteasome. In at least one embodiment, the at least one fourth agent inhibits the activity of one or more inflammasome or infectosome. Infectosomes are utilized in the maturation cleavage of particular infective agents, including viruses, while inflammasomes are generally involved in inflammatory reactions, including activation of particular caspases, interleukins, or other cytokines.

Proteasome inhibitors include peptide aldehydes, peptide vinyl suflones, peptide boronates, peptide epoxyketones, and P-lactones. Without wishing to be bound by any particular theory of mechanism, the proteasome inhibitors are classified based on the pharmacophore that reacts with a threonine residue in the active site of the proteasome. The proteasome inhibitor bortezomib has been used for the treatment of relapsed multiple myeloma. (See e.g., Cvek and Dvorak, Curr. Pharm. Design, Vol. 13, pp. 1-13 (2007), which is herein incorporated by reference). It has also been shown that dithiocarbamates complexed with metals (e.g., copper or zinc) are selectively toxic to melanoma cells in the presence of normal cells. (See e.g., Cvek and Dvorak, Curr. Pharm. Design, Vol. 13, pp. 1-13 (2007), which is herein incorporated by reference). In at least one embodiment, the at least one agent configured to modulate the activity of at least one proteasome includes dichloroisocoumarin or bortezomib.

Whole-body inflammation that is caused by infection is generally divided into systemic inflammatory response syndrome, sepsis, septic shock, and multiple organ dysfunction syndrome. Systemic inflammatory response syndrome is usually treated with fluids and possibly antibiotics. If left untreated, or if symptoms are not responsive to treatment, severe sepsis can occur that leads to organ dysfunction, low blood pressure, or insufficient blood flow to one or more organs. Sepsis can also lead to septic shock, multiple organ failure, and death. (See e.g., Remick, Curr. Pharm. Design, pp. 1-8, 2003, which is herein incorporated by reference). Without wishing to be bound by any particular theory, one of the underlying causes of sepsis and septic shock is believed to be an unregulated increase in inflammatory cytokines in the subject's body. Some examples of inflammatory cytokines that may be involved with this type of inflammation include but are not limited to increases in IL-1, IL-6, IL-18, and tumor necrosis factor (TNF).

Malaria is a parasitic infection by plasmodium, primarily of erythrocytes. Typically, the rupture of parasitized erythrocytes results in systemic release of proinflammatory cytokines that leads to an onset of symptoms of fever and rigors. (See e.g., Parroche et al., PNAS, Vol. 104, No. 6, pp. 1919-1924 (2007), which is herein incorporated by reference). Without wishing to be bound by any particular theory, it is believed that during the intraerythrocyte stage, parasites digest hemoglobin in the food vacuole. The resulting potentially toxic heme metabolites are detoxified by the parasite by conversion to an insoluble crystal of hemozoin. (See e.g., Parroche et al., PNAS, Vol. 104, No. 6, pp. 1919-1924 (2007), which is herein incorporated by reference). Hemozoin is generally cleared from the blood of infected subjects by blood circulation through the liver and spleen. It is also believed that hemozoin binds plasmodial DNA, which activates one or more Toll-like receptors, and at least Toll-like receptor 9. (See e.g., Parroche et al., PNAS, Vol. 104, No. 6, pp. 1919-1924 (2007), which is herein incorporated by reference). Toll-like receptor 9 has been described as a receptor for DNA, including unmethylated CpG-containing DNA from bacteria or other microorganisms.

In addition, it is believed that the glycosylphosphatidylinositol anchors from protozoan infections, as well as other parasitic infections, activate one or more Toll-like receptors (TLRs). In human disease, polymorphisms in TLRs 2, 4, and 9 affect outcome of malaria infection. In addition, MyD88-null mice have a decreased production of IL-12 and less severe pathology than wild type control mice. (See e.g., Parroche et al., PNAS, Vol. 104, No. 6, pp. 1919-1924 (2007), which is herein incorporated by reference).

The DNA ligands for Toll-like receptor 9 have been categorized in three classes, A, B, and C. The A class of oligonucleotides generate a strong Type I interferon response, while the B class of oligonucleotides do not. The C class of olignucleotides appear to be an intermediary class. (See e.g., Parroche et al., PNAS, Vol.104, No. 6, pp. 1919-1924 (2007), which is herein incorporated by reference).

The majority of CpG motifs in the malaria genome appear to possess a B class motif, with only a few A class or C class CpG motifs. Oligonucleotides based on malaria CpG-rich motifs are highly immunostimulatory, and are believed to be activators of Toll-like receptor 9. (See e.g., Parroche et al., PNAS, Vol. 104, No. 6, pp. 1919-1924 (2007), which is herein incorporated by reference).

In at least one embodiment, a therapeutic composition as described herein is configured to modulate the production or activity of at least one cytokine. In at least one embodiment, a therapeutic composition as described herein is configured to inhibit the production or activity of at least one cytokine. In at least one embodiment, the at least one cytokine includes one or more members of the α-helix bundle cytokine family. In at least one embodiment, a therapeutic composition modulates the production of one or more of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, IL-39, IL-40, IL-41, IL-42, IFN-γ, IFN-α, IFN-β, or TNF-α.

Chemokines are biochemical signaling molecules that act to attract other particular molecules, including but not limited to cells, to a specific site. In at least one embodiment, a therapeutic composition is configured to modulate the production or activity of one or more chemokines. In at least one embodiment, the one or more chemokines include at least one of a CC chemokine, CXC chemokine, C chemokine, or CX3C chemokine. In at least one embodiment, the one or more chemokines include at least one of CCL1, CCL2, CCL3, CCL4, CCL5, CCL6, CCL7, CCL8, CCL9/CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CCL29, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, CXCL18, CXCL19, CXCL20, CXCL21, CXCL22, XCL1, XCL2, XCL3, XCL4, XCL5, CX3CL1, CX3CL2, CX3CL3.

In at least one embodiment, a therapeutic composition also includes at least one of sulfadoxine-pyrimethamine, mefloquine, doxycycline, atovaquone-proguanil, artemether, arteether, artelinic acid, artemotil, dihydroartemisin, dihydroartemisin-piperaquine, amodiaquine, lumefantrine, artesunate, artemisinin, or primaquine.

Any of the therapeutic compositions described herein include formulations for administration to a subject by at least one route, including but not limited to peroral, oral, topical, transdermal, epidermal, intravitreal, transmucosal, inhalation, parenteral, enteral, or injection. The delivery may include inhalation, depot injections, implants, or other mode of delivery by way of an apparatus.

Any of the therapeutic compositions described herein include formulations for administration to at least one subject. In at least one embodiment, a therapeutic composition includes a time-release formulation. In at least one embodiment, a therapeutic composition includes at least one solid, liquid, or gas. In at least one embodiment, a therapeutic composition includes at least one of an aerosol, gel, sol, ointment, solution, suspension, capsule, tablet, cachets, suppository, cream, device, paste, liniment, lotion, ampule, elixir, emulsion, microemulsion, spray, suspension, powder, syrup, tincture, detection material, polymer, biopolymer, buffer, adjuvant, diluent, lubricant, disintegration agent, suspending agent, solvent, colorant, glidant, anti-adherent, anti-static agent, surfactant, emulsifying agent, flavor, gum, sweetener, coating, binder, filler, compression aid, encapsulation aid, plasticizer, preservative, granulation agent, spheronization agent, stabilizer, adhesive, pigment, sorbent, or nanoparticle.

The formulation of any of the therapeutic compositions described herein may be formulated neat or may be combined with one or more acceptable carriers, diluents, excipients, and/or vehicles such as, for example, buffers, surfactants, preservatives, solubilizing agents, isotonicity agents, and stablilizing agents as appropriate. A “pharmaceutically acceptable” carrier, for example, may be approved by a regulatory agency of the state and/or Federal government such as, for example, the United States Food and Drug Administration (US FDA) or listed in the U.S. Pharmacopeia or other generally recognized pharmacopeia for use in animals, and more particularly in humans. Conventional formulation techniques generally known to practitioners are described in Remington: The Science and Practice of Pharmacy, 20^{th} Edition, Lippincott Williams & White, Baltimore, Md. (2000), which is herein incorporated by reference.

Acceptable pharmaceutical carriers include, but are not limited to, the following: sugars, such as lactose, glucose and sucrose; starches, such as corn starch and potato starch; cellulose, and its derivatives, such as sodium carboxymethyl cellulose, ethyl cellulose, cellulose acetate, and hydroxymethylcellulose; polyvinylpyrrolidone; cyclodextrin and amylose; powdered tragacanth; malt; gelatin, agar and pectin; talc; oils, such as mineral oil, polyhydroxyethoxylated castor oil, peanut oil, cottonseed oil, safflower oil, sesame oil, olive oil, corn oil and soybean oil; polysaccharides, such as alginic acid and acacia; fatty acids and fatty acid derivatives, such as stearic acid, magnesium and sodium stearate, fatty acid amines, pentaerythritol fatty acid esters; and fatty acid monoglycerides and diglycerides; glycols, such as propylene glycol; polyols, such as glycerin, sorbitol, mannitol and polyethylene glycol; esters, such as ethyl oleate and ethyl laurate; buffering agents, such as magnesium hydroxide, aluminum hydroxide and sodium benzoate/benzoic acid; water; isotonic saline; Ringer's solution; ethyl alcohol; phosphate buffer solutions; other non-toxic compatible substances employed in pharmaceutical compositions. The pharmaceutical compositions are generally formulated as sterile, substantially isotonic and in full compliance with all Good Manufacturing Practice (GMP) regulations of the U.S. Food and Drug Administration.

Table I is a non-limiting table of therapeutic agents that are combined as described herein to formulate at least one therapeutic composition.

TABLE I

| Toll-Like | Src family | | | |
|---|---|---|---|---|
| receptor | kinase | NF-kB | Protease | Proteasome |
| inhibitors | inhibitors | inhibitors | inhibitors | inhibitors |
| Chloroquine | Dasatinib | Disulfiram | Saquinavir | Dichloro- |
| | | | | isocoumarin |
| Quinine | Nilotinib | Ditiocarb | Ritonavir | Bortezomib |
| M62812 | BMS- | Sulindac | Indinavir | |
| | 268770 | | | |
| | UR-12947 | Sulfasalazine | Nelfinavir | |
| | Aztreonam | Bortezomib | Amprenavir | |
| | MZ-338 | | Lopinavir | |
| | Riluzole | | Atazanavir | |
| | Meloxicam | | Fosamprenavir | |
| | Pramipexole | | Tipranavir | |
| | CBS-113-A | | Darunavir | |
| | AZD0530 | | | |
| | INNO-406 | | | |
| | MK-0457 | | | |
| | Cediranib | | | |
| | Sunitinib | | | |
| | Bosutinib | | | |
| | Axitinib | | | |
| | Erlotinib | | | |
| | Gefitinib | | | |
| | Lapatinib | | | |
| | Lestaurtinib | | | |
| | Semaxanib | | | |
| | Imatinib | | | |

Additionally, in an embodiment, the one or more of the following therapeutic agents are added as described herein, particularly for treatment of malaria or other inflammatory diseases or conditions: sulfadoxine-pyrimethamine, mefloquine, doxycycline, atovaquone-proguanil, artemether, arteether, artelinic acid, artemotil, dihydroartemisin, dihydroartemisin-piperaquine, amodiaquine, lumefantrine, artesunate, artemisinin, or primaquine.

At least one embodiment disclosed herein includes one or more methods for modulating at least one immune response of one or more cells by contacting the one or more cells with an effective amount of at least one therapeutic composition described herein.

In at least one embodiment, the one or more cells are located at least in one of in vitro, in vivo, in situ, in utero, or ex vivo. In at least one embodiment, the one or more cells are located in a subject, wherein the subject is afflicted with or suspected of being afflicted with at least one inflammatory disease or condition. As described herein, the at least one inflammatory disease or condition may include one or more of a pathogenic infection, parasitic infection, autoimmune disease, sepsis, systemic inflammatory response syndrome, septic shock, multiple organ dysfunction syndrome, allergic reaction, or cancer. In at least one embodiment, the at least one inflammatory disease or condition includes one or more of anaphylaxis, viral infection, bacterial infection, plasmodium infection, protozoan infection, nematode infection, or other worm infection. In at least one embodiment, the at least one inflammatory disease or condition includes malaria. In at least one embodiment, the parasitic infection includes at least one infection or infestation of one or more of a phytoparasite, zooparasite, ectoparasite, endoparasite, or one or more of parasitic cysts, larvae, or eggs.

In at least one embodiment, the one or more methods relating to modulating at least one immune response of one or more cells reduces inflammation. In at least one embodiment, the one or more methods relating to modulating at least one immune response of one or more cells reduces or ameliorates at least one sign or symptom of inflammation.

In at least one embodiment, one or more methods relate to modulating at least one immune response of one or more cells further includes detecting in the subject at least one level of at least one biological signaling molecules that is associated with at least one inflammatory disease or condition. Biological signaling molecules may include, but not be limited to, one or more of a nucleic acid, amino acid, peptide, polypeptide, protein, carbohydrate, lipid, glycoprotein, glycopeptide, glycolipid, lipopolysaccharide, metalloprotein, or proteoglycan. In at least one embodiment, the at least one biological signaling molecule includes one or more of a cytokine, chemokine, cellular receptor, intracellular second messenger, protease, kinase, enzyme, cellular receptor ligand, transcription factor, or hormone.

In at least one embodiment, a therapeutic composition includes at least two agents that are configured to modulate an immunological reaction. Multiple immunological reactions occur in relation to an inflammatory disease or condition in a subject, including but not limited to a humoral response, a cell mediated response, an innate response, an immune tolerance response, an autoimmune response, a hyperimmune response, or a hypersensitivity response.

At least one embodiment relates to one or more methods of modulating the activity of intracellular signaling molecules. In an embodiment, a method relates to modulating the activity of one or more Toll-like receptors and one or more Src family kinases by administering to the subject at least one of the therapeutic compositions described herein.

At least one embodiment relates to one or more methods of modulating the activity of one or more Toll-like receptors and one or more NF-kB molecules by administering to the subject at least one of the therapeutic compositions described herein containing at least one agent configured to modulate the activity of one or more Toll-like receptors and at least one agent configured to modulate the activity of one or more NF-kB molecules.

At least one embodiment relates to one or more methods of modulating the activity of one or more Toll-like receptors and one or more Src family kinases by administering to the subject at least one of the therapeutic compositions described herein containing at least one agent configured to modulate the activity of one or more Toll-like receptors and at least one agent configured to modulate the activity of one or more Src family kinases.

At least one embodiment relates to one or more methods of modulating the activity of one or more NF-kB molecules and one or more Src family kinases by administering to the subject at least one of the therapeutic compositions described herein containing at least one agent configured to modulate the activity of one or more NF-kB molecules and at least one agent configured to modulate the activity of one or more Src family kinases.

At least one embodiment relates to one or more methods of modulating the activity of one or more Toll-like receptors, one or more Src family kinases, and one or more NF-kB molecules by administering to the subject at least one of the therapeutic compositions described herein containing at least one agent configured to modulate the activity of one or more Toll-like receptors and at least one agent configured to modulate the activity of one or more Src family kinases, and at least one agent configured to modulate the activity of one or more NF-kB molecules.

Any of the methods disclosed herein may include detecting in the subject, or tissues, at least one level of at least one biological signaling molecule that is associated with an immulogical response or that is associated with at least one inflammatory disease or condition.

Detection of one or more of the biological signaling molecules can be by any method known in the art, including but not limited to analyzing one or more biological tissues or fluids from the subject. Analyzing one or more biological fluids can be performed by any of a variety of methods known in the art, including but not limited to utilizing one or more of thin-layer chromatography, mass spectrometry, nuclear magnetic resonance, polymerase chain reaction, reverse transcriptase, Northern blot, Western blot, microscopy, flow cytometry, antibody binding, enzyme-linked immunosorbent assay, radioactive absorption or release, microfluidic analysis, nucleic acid chip array analysis, protein chip array analysis, chemical sensor analysis (including arrays), biosensor analysis, cell counting, or cell sorting.

In at least one embodiment, the at least one biological signaling molecule includes but is not limited to, one or more nucleic acid, amino acid, peptide, polypeptide, protein, glycopeptide, glycoprotein, glycolipid, lipopolysaccharide, peptidoglycan, proteoglycan, lipid, metalloprotein, liposome, or carbohydrate. Carbohydrates may include, but not be limited to, oligosaccharides, glycans, glycosaminoglycans, or derivatives thereof.

In at least one embodiment, the at least one biological signaling molecule includes but is not limited to at least one cytokine, chemokine, cellular receptor, intracellular second messenger, protease, kinase, enzyme, cellular receptor ligand, transcription factor, or hormone.

Modulators include activators and inhibitors. Modulating can increase or decrease a biological response in a manner that activates or inhibits an inflammatory reaction. Activators are agents that, e.g., bind to, stimulate, increase, open, activate, facilitate, enhance activation, sensitize or up-regulate the activity of a particular molecule related to inflammation (e.g. agonists). Inhibitors are agents that, e.g., bind to, partially or totally block stimulation, decrease, prevent, delay activation, inactivate, desensitize, or down-regulate the activity of a steroid hormone intermediate, a receptor, or a steroid hormone receptor, e.g., antagonists. Modulating a response includes altering the response by way of e.g., proteins that bind activators or inhibitors, receptors, genetically modified versions of naturally-occurring ligands or receptors, or other molecules that alter the activity of specific molecules.

In at least one embodiment, the one or more cells are located in at least one subject. A subject includes, but is not limited to, a vertebrate or invertebrate, including a fish, reptile, mammal, amphibian, or bird. In at least one embodiment, the subject includes at least one human.

A treatment regimen may include a therapeutic amount of one or more therapeutic compositions described herein that includes modulators or analogs thereof. The treatment regimen may further include a schedule of changes in the dosage of the therapeutic composition to maintain a desired level of one or more molecules related to inflammation in one or more tissues or subjects. Such treatment may be individualized for the tissue or subject. Treating or treatment that includes administration of at least one of the therapeutic compositions included herein may prevent or delay the onset of symptoms, complications, or biochemical indicia of a disease or condition, alleviate the symptoms, arrest, or inhibit further development of the disease, condition, or disorder. Treatment or administration of at least one therapeutic composition described herein may be prophylactic to prevent or delay the onset of a disease or condition, or prevent the manifestation of clinical or subclinical symptoms thereof, or therapeutic suppression or alleviation of symptoms after the manifestation of the disease.

A treatment regimen may be continuous and uninterrupted, which indicates that there is no break in the treatment regimen during the treatment period. Continuous, uninterrupted administration of a combinational therapeutic composition includes that the combination may be administered during the entire treatment period, e.g., at least once daily or on a continuous and uninterrupted basis. The treatment regimen may be given to maintain an in vivo therapeutic level or a determined cyclic level of the one or more agents of the at least one therapeutic composition.

It is expected that the treatment period may vary depending, for example, on the symptoms to be treated. Physician evaluation along with patient interaction will assist in the determination of the duration of treatment. Adjustments in the treatment regimen may depend upon the individual's medical history, or genetic or proteomic information.

At least one embodiment relates to one or more methods based on a genetic or proteomic profile of the subject. Medical evaluation regarding genetic profiling or genetic testing can be provided as a current determination of genetic risk factors, or as part of the subject's medical history. Genetic profiling or genetic testing can be used to design a treatment regimen and thus determine an optimal level individualized for the subject. A physician may use the genetic profile or genetic testing information to determine a genetic basis for needed treatment based on baseline or physiological levels of inflammatory agents.

Prior to determining a treatment regimen, additional information can be obtained regarding any particular inflammatory disease or condition in relation to any possible therapeutic treatment derived from population databases. The medical evaluation can include information in a population database on disease risks, available drugs and formulations, and documented population responses to drugs and formulations.

In at least one embodiment, one or more polymorphisms are determined prior to administration of at least one therapeutic composition described herein, which could allow for such therapeutic composition to be tailored to a particular subject's genetic makeup. In at least one embodiment, the therapeutic composition modulates the activity of one or more Toll-like receptors, one or more Src family kinases, or one or more NF-kB molecules that are produced by at least one polymorphism.

In at least one embodiment, the therapeutic compositions and methods described herein modulate one or more specific Toll-like receptors, Src family kinases, or NF-kB molecules that are the result of a particular polymorphism in a tissue or subject.

In at least one embodiment, methods disclosed herein relate to treating a subject afflicted with or suspected of being afflicted with at least one inflammatory disease or condition by administering to the subject an effective amount of a therapeutic composition disclosed herein. Certain aspects of inflammatory diseases or conditions include, but are not limited to, an inflammatory condition or disease state at a particular time, including an atypical inflammatory condition for a subject or tissue. The caustive agent or agents may or may not be known, and can include pathogenic infection or infestation such as by a microorganism or small molecule, including but not limited to a viruses, bacteria, parasites, or infectious proteins, prions, virons or viroids. In at least one embodiment, the subject is afflicted with or suspected of being afflicted with malaria.

In at least one embodiment, methods disclosed herein relate to treating a subject afflicted with or suspected of being afflicted with malaria, including administering to the subject an effective amount of at least one therapeutic composition including at least one of chloroquine, M62812, or quinine, at least one of dasatinib, nilotinib, BMS-268770, UR-12947, aztreonam, MZ-338, riluzole, meloxicam, pramipexole, CBS-113-A, AZD0530, bosutinib, INNO-406, MK-0457, or imatinib; and at least one pharmaceutically-acceptable carrier or excipient. In at least one embodiment, the therapeutic composition further includes at least one of disulfiram, ditiocarb, sulindac, sulfasalazine, or bortezomib. In at least one embodiment, the therapeutic composition further includes Cathepsin K. In at least one embodiment, the therapeutic composition further includes dichlorisocoumarin or bortezomib. In at least one embodiment, the therapeutic composition further includes at least one of sulfadoxine-pyrimethamine, mefloquine, doxycycline, atovaquone-proguanil, artemether, arteether, artelinic acid, artemotil, dihydroartemisin, dihydroartemisin-piperaquine, amodiaquine, lumefantrine, artesunate, artemisinin, or primaquine.

The inflammatory disease or condition may be clinically diagnosed disease or the organism may be suspected of being afflicted with at least one inflammatory disease or condition based on the signs or symptoms of subject's disease state or condition, or physiological baseline.

In conjunction with the at least one inflammatory disease or condition, there may be at least one responsive state in the subject or its tissue or tissues. The responsive state may include but not be limited to an immune response, an inflammatory response, a hyperimmune response, hypersensitive response, allergic response, or an autoimmune response.

In at least one embodiment, a method of treating a subject afflicted with or suspected of being afflicted with at least one inflammatory disease or condition with at least one therapeutic composition described herein, including at least one of chloroquine, M62812, or quinine; at least one of dasatinib, nilotinib, BMS-268770, UR-12947, aztreonam, MZ-338, riluzole, meloxicam, pramipexole, CBSS-113-A, AZD0530, bosutinib, INNO-406, MK-0457, or imatinib; and at least one pharmaceutically-acceptable carrier or excipient. In at least one embodiment, the therapeutic composition also includes at least one of disulfiram, ditiocarb, sulindac, sulfasalazine, or bortezomib. In at least one embodiment, the therapeutic composition further includes Cathepsin K. In at least one embodiment, the therapeutic composition includes at least one of dichloroisocoumarin or bortezomib. In at least one embodiment, the therapeutic composition further comprises at least one of sulfadoxine-pyrimethamine, mefloquine, doxycycline, atovaquone-proguanil, artemether, arteether, artelinic acid, artemotil, dihydroartemisin, dihydroartemisin-piperaquine, amodiaquine, lumefantrine, artesunate, artemisinin, or primaquine.

As set forth herein, the compositions disclosed are formulated by standard practice. In certain instances, in order to account for bioavailability, a formulation may be provided in rapid release, extended release or slow-release form prior to administration. Likewise, liposomes, microsomes, or other vehicles or composition modifications allow for regulating the dosage by increasing or decreasing the rate of composition delivery, maintenance, decomposition, clearance, or other factors. For example, one particular therapeutic agent may have bioavailability properties that require it to be modified by standard techniques so that it can be administered simultaneously with another therapeutic agent. Similarly, in the instance where multiple therapeutic agents are included in a single composition, it may be necessary to modify one or more of the therapeutic agents by standard techniques.

In at least one embodiment the one or more biological signaling molecules are detected by one or more recognition molecules specific to the one or more biological signaling molecules. The recognition molecules may include, but not be limited to, an antibody, affibody, DNA-recognition molecule, aptamer, or other molecule.

An antibody may include an anti-idiotypic antibody, a heteroantibody, multiple antibodies, one or more antibody fragments, one or more antibody derivatives, one or more antibodies linked together, chimeric antibodies, humanized antibodies, human antibodies, recombinant antibodies, synthetic antibodies, or others.

Antibodies or fragments thereof may be generated against an agent, such as a receptor or ligand, using standard methods, for example, such as those described by Harlow & Lane (Antibodies: A Laboratory Manual, Cold Spring Harbor Laboratory Press; 1^{st} edition 1988), which is herein incorporated by reference). Alternatively, an antibody fragment directed against an agent may be generated using phage display technology (See, e.g., Kupper, et al. BMC Biotechnology Vol. 5, No. 4, (2005), which is herein incorporated by reference). An antibody or fragment thereof could also be prepared using in silico design (See e.g., Knappik et al., J. Mol. Biol. Vol. 296, pp. 57-86 (2000), which is herein incorporated by reference). In addition or instead of an antibody, the assay may employ another type of recognition element, such as a receptor or ligand binding molecule. Such a recognition element may be a synthetic element like an artificial antibody or other mimetic. (See e.g., U.S. Pat. No. 5,804,563 (Synthetic receptors, libraries and uses thereof), U.S. Pat. No. 6,797,522 (Synthetic receptors), U.S. Pat. No. 6,670,427 (Template-textured materials, methods for the production and use thereof), and U.S. Pat. No. 5,831,012, U.S. Patent Application 20040018508 (Surrogate antibodies and methods of preparation and use thereof); and Ye and Haupt, Anal Bioanal Chem. Vol. 378, pp. 1887-1897, (2004); Peppas and Huang, Pharm Res. Vol. 19, pp. 578-587 (2002), each of which is herein incorporated by reference).

In some instances, antibodies, recognition elements, or synthetic molecules that recognize a Toll-like receptor, Src family kinase, or NF-kB molecule may be available from a commercial source, e.g., Affibody® affinity ligands (See e.g., Abcam, Inc. Cambridge, Mass. 02139-1517; U.S. Pat. No. 5,831,012, incorporated here in by reference).

In some instances, levels of particular biological signaling molecules may be assayed in a bodily fluid or tissue using gas or liquid chromatography with or without mass spectrometry. A bodily fluid may include blood, lymph, saliva, urine, sweat, ascites, serum, urogenital secretion, bone marrow, a tissue secretion or excretion, or other fluid.

A level of one or more biological signaling molecules may also be assayed in a bodily fluid or tissue using a recombinant cell based assay or sensor. A sensor may include, for example a chemical sensor, biosensor, protein array, or microfluidic device.

Prior to determining a treatment regimen, additional information regarding the physiological status of the subject or tissue may be gathered and assessed. For example, information may be collected on a subject's medical history or familial history, including genetic or proteomic information. The individualized medical evaluation can include a genetic profile of the subject regarding genes, genetic mutations or genetic polymorphisms that indicate risk factors that affect disease related to Toll-like receptors, Src family kinases, or NF-kB molecules.

A genetic polymorphism or genetic mutation in a genetic profile of a subject that encodes a component of one or more Toll-like receptors, Src family kinases, or NF-kB molecules may affect the levels of such molecules. Thus, genetic profiling may be used prior to the initiation of a treatment regimen including providing one or more agents that modulate one or more Toll-like receptors, Src family kinases, or NF-kB molecules, in order to assess whether the subject or tissue has any genetic mutations or genetic polymorphisms that may be correlated with a particular immune or inflammatory response.

A genetic polymorphism or mutation may indicate how a tissue or subject will respond to a particular treatment regimen. Genomic DNA used in genetic profiling may be isolated from any biological sample which contains the DNA of that subject or tissue, including but not limited to blood, saliva, cheek swab, epithelium, or other tissue. For example, genomic DNA may be extracted from whole blood or from isolated peripheral blood leukocytes isolated by differential centrifugation from whole blood using a commercial kit (See e.g., QIAmp DNA Blood Mini Kit, Qiagen, Valencia, Calif.) according to the manufacturer's instructions.

Medical evaluation of the subject or tissue for genetic or proteomic profiling or genetic or proteomic testing may be provided as a current determination of genetic risk factors in the subject or tissue, or as part of the subject's medical history. Genetic profiling or genetic testing may be determined by using a variety of methods including but not limited to restriction landmark genomic scanning (RLGS), Southern blot analysis combined with restriction fragment length polymorphism (RFLP), fluorescence in situ hybridization (FISH), enzyme mismatch cleavage (EMC) of nucleic acid heteroduplexes, ligase chain reaction (LCR) or polymerase chain reaction (PCR) based methods. Analysis of one or more single nucleotide polymorphisms (SNPs) may also be used for genetic profiling.

Restriction fragment landmark genomic scanning (RLGS) may be used to scan an entire mammalian genome. As such, genomic DNA is digested with restriction enzymes to generate large DNA fragments. The fragments are separated on an agarose gel, digested with one or more restriction enzymes within the agarose gel, and then separated in a second dimension by polyacrylamide gel electrophoresis (PAGE) (See e.g., Tawata, et al., Comb. Chem. High Throughput Screen. Vol. 3, pp. 1-9 (2000), which is herein incorporated by reference). The DNA may be labeled prior to digestion, or the fragments may be stained nonspecifically as with an intercalating dye, for example. The resulting pattern may be compared with pre-established norms to detect genetic mutations.

Restriction fragment length polymorphism (RFLP) is similar to restriction fragment landmark genomic scanning in that the genomic DNA is digested with specific restriction enzymes and separated on an agarose gel. The separated DNA is transferred to a membrane and the fragments are visualized using hybridization analysis and gene specific probes.

A variety of PCR related methods may be used for genetic profiling and may be used to detect both known and unknown mutations and polymorphisms (See e.g., Tawata, et al., Comb. Chem. High Throughput Screen. Vol. 3, pp. 1-9 (2000), which is herein incorporated by reference). For known mutations and polymorphisms, specific PCR oligonucleotide probes are designed to bind directly to the mutation or polymorphism or proximal to the mutation or polymorphism. For example, PCR may be used in combination with RFLP. In this instance, a DNA fragment or fragments generated by PCR with primers on either side of the mutation or polymorphism site are treated with restriction enzymes and separated by agarose gel electrophoresis. The fragments themselves may be detected using an intercalating dye such as, for example, ethidium bromide. An aberrant banding pattern may be observed if mutations exist within the restriction sites. PAGE may be used to detect single base differences in the size of a fragment.

Alternatively, PCR may be used in combination with DNA sequencing for genetic profiling. For example, PCR primers may be designed that bind to either side of a potential mutation site on the target DNA and generate a PCR fragment that spans a potential mutation site. The PCR fragment is either directly sequenced or subcloned into a cloning vector and subsequently sequenced using standard molecular biology techniques.

Alternatively, a mutation or polymorphism may be screened using comparative genomic hybridization (CGH) (See e.g., Pinkel & Albertson, Nat. Gen. Vol. 37:S11-S17 (2005), which is herein incorporated by reference). In this instance, “normal” genomic DNA and test genomic DNA are differentially labeled and hybridized to metaphase chromosomes or DNA microarrays. The relative hybridization signal at a given location is proportional to the relative copy number of the sequences in the reference and test genomes. Arrays may be generated using DNA obtained from, for example, bacterial artificial chromosomes (BACs) or PCR.

Analysis of one or more single nucleotide polymorphism (SNP) may be used for genetic profiling. A SNP is a DNA sequence variation in which a single nucleotide in the genomic sequence differs between members of a species (or between paired chromosomes of an individual). For a variation to be considered a SNP it must occur in at least 1% of the population. Most SNPs do not affect protein function, and/or are not responsible for a disease state, but they may serve as biological markers for pinpointing an altered protein or disease on the human genome map as they are often located near a gene found to be associated with a certain disease. Occasionally, a SNP may actually affect protein function and/or cause a disease and, therefore, can be used to search for and isolate a specific gene, e.g., a T to C mutation in the CYP17 gene which affects enzyme function. The pattern of SNPs in a subject's genomic DNA may be compared with information in databases in an association study to determine effect on protein function and/or risk of disease development. SNPs may be identified using PCR and DNA sequencing as described above. Alternatively, SNP genotyping may be done using high throughput array analysis (See e.g., Applied BioSystems, ABI PRISM, 3100 Genetic Analyzer with 22-cm Capillary Array; Syvanen, et al., Nat. Genet., Vol. 37, pp. S5-S10(2005) which is herein incorporated by reference). A growing number of web-based databases are available for finding information regarding SNPs and protein function and/o disease associations (See e.g., International HapMap Project on the worldwide web at //snp.csh1.org; Nature 449: 851-861, 2007; National Center Biotechnology Information (NCBI) Single Nucleotide Polymorphisms, on the worldwide web at ncbi.nlm.nih.gov/projects/SNP/, which is herein incorporated by reference).

In certain instances, such as malaria, it is believed that the genetic mutations resulting in G6PD deficiency, α+ thalassemia, and hemoglobin C in humans are positively selected in areas with high incidence of malaria infection. (See, e.g., Kwiatkowski, Am. J. Hum. Gen. Vol. 77, pp. 171-190, (2005), which is herein incorporated by reference). One particular example of an evolutionary protection against malaria infection is the HBB gene, in which three different coding SNPs confer protection against malaria: Glu6Val (HbS), Glu6Lys (HbC), and Glu26Lys (HbE). While homozygotes for the HbS gene suffer from sickle-cell disease, heterozygotes have a ten-fold reduced risk of severe malaria. (See, e.g., Kwiatkowski, Am. J. Hum. Gen. Vol. 77, pp. 171-190, (2005), which is herein incorporated by reference). The HbS allele is common in Africa but rare in Southeast Asia, whereas the opposite is true for the HbE allele. However, even at local levels, there are different levels of HbS, HbC, and HbE variants. (See, e.g., Kwiatkowski, Am. J. Hum. Gen. Vol. 77, pp. 171-190, (2005), which is herein incorporated by reference). It is believed that many genetic factors of the subject may interact with environmental variables, as well as parasitic genetic factors, in determining a particular subject's susceptibility or resistance to the malaria parasite.

The disclosure further provides kits including at least one therapeutic composition or method disclosed herein. Any particular kit may also contain instructional material teaching the methodologies and uses of the therapeutic composition or method, as described herein.

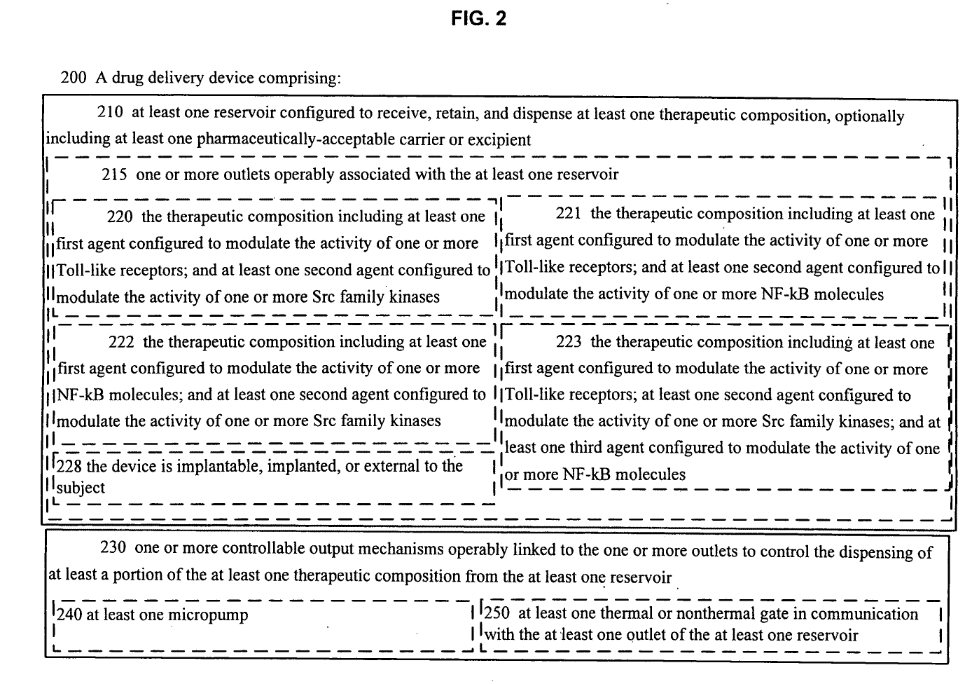

With reference to the figures, FIG. 2 illustrates a drug delivery device 200 including at least one reservoir 210 configured to receive, retain, and dispense at least one therapeutic composition. Any number of delivery devices may be utilized for delivery of the therapeutic compositions described herein. For example, devices described in U.S. patent application Ser. No. 11/975347, which is herein incorporated by reference, can be employed.

In an embodiment, the therapeutic composition 220 includes at least one first agent configured to modulate the activity of one or more Toll-like receptors; at least one second agent configured to modulate the activity of one or more Src family kinases; and at least one pharmaceutically acceptable carrier or excipient.

In an embodiment, the therapeutic composition 221 includes at least one first agent configured to modulate the activity of one or more Toll-like receptors; at least one second agent configured to modulate the activity of one or more NF-kB molecules; and at least one pharmaceutically acceptable carrier or excipient.

In an embodiment, the therapeutic composition 222 includes at least one first agent configured to modulate the activity of one or more NF-kB molecules; at least one second agent configured to modulate the activity of one or more Src family kinases; and at least one pharmaceutically acceptable carrier or excipient.

In an embodiment, the therapeutic composition 223 includes at least one first agent configured to modulate the activity of one or more Toll-like receptors; at least one second agent configured to modulate the activity of one or more Src family kinases; at least one third agent configured to modulate the activity of one or more NF-kB molecules; and at least one pharmaceutically acceptable carrier or excipient.

In at least one embodiment, the device includes one or more controllable output mechanisms 230 operably linked to the one or more outlets to control the dispensing of at least a portion of the at least one therapeutic composition (220, 221, 222, or 223) from the at least one reservoir (210). The controllable output mechanism 230 may include at least one micropump 240 or at least one thermal or nonthermal gate 250 in communication with the at least one outlet of the at least one reservoir 210.

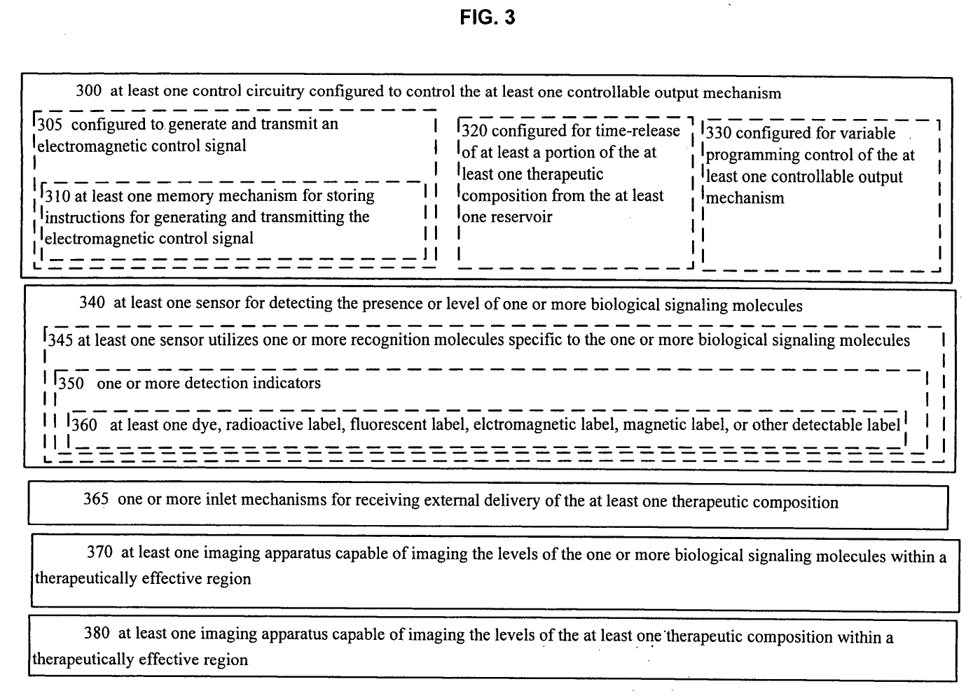

As illustrated in FIG. 3, the drug delivery device 200 may further include at least one control circuitry 300 configured to control the at least one controllable output mechanism 230. In at least one embodiment, the at least one control circuitry 300 is configured to generate and transmit an electromagnetic control signal 305 and may contain at least one memory mechanism 310 for storing instructions for generating and transmitting the electromagnetic control signal. In an embodiment, the at least one controllable output mechanism 300 may be configured for time-release 320 of at least a portion of the at least one therapeutic composition (220, 221, 222, or 223) from the at least one reservoir. In at least one embodiment, the at least one control circuitry 300 can be configured for variable programming control 330.

In at least one embodiment, the device can include at least one first sensor 340 for detecting the presence or level of one or more biological signaling molecules. As described herein, detecting the presence or level of one or more biological signaling molecules may include utilizing one or more recognition molecules 345 specific to the one or more biological signaling molecules. Biological signaling molecules, as well as recognition molecules are described herein.

In at least one embodiment, the at least one sensor for detecting the presence or level of one or more biological signaling molecules includes one or more detection indicators 350. In at least one embodiment, the one or more detection indicators 350 include at least one dye, radioactive label, fluorescent label, electromagnetic label, magnetic label, or other detectable label 360. In at least one embodiment, the drug delivery device includes one or more inlet mechanisms 365 for receiving external delivery of the at least one therapeutic composition. In at least one embodiment, the device includes at least one imaging apparatus 370 capable of imaging the levels of the one or more biological signaling molecules within a therapeutically effective region. In at least one embodiment, the device includes at least one imaging apparatus 380 capable of imaging the levels of the at least one therapeutic composition within a therapeutically effective region.

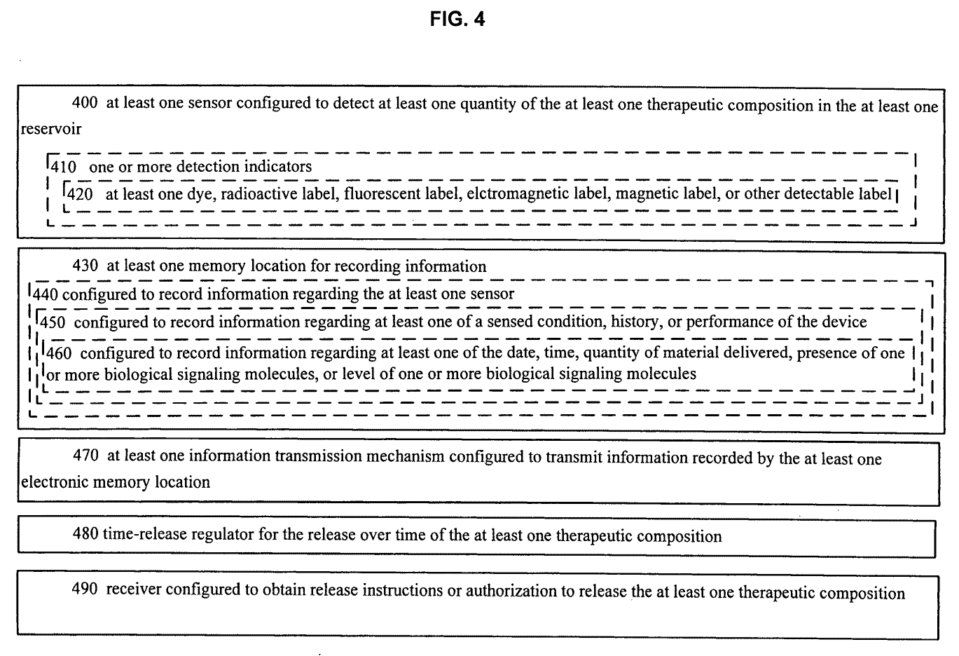

As indicated in FIG. 4, in at least one embodiment, the device may include at least one second sensor 400 configured to detect at least one quantity of the at least one therapeutic composition (220, 221, 222, or 223) in the at least one reservoir 210. In at least one embodiment, the sensor 400 includes one or more detection indicators 410. In at least one embodiment, the one or more detection indicators 410 include at least one dye, radioactive label, fluorescent label, electromagnetic label, magnetic label, or other detectable label 420. In at least one embodiment, the at least one second sensor 400 and the at least one first sensor 340, are the same sensor. In at least one embodiment, the device further includes at least one memory location 430 for recording information. In at least one embodiment, the at least one memory location 430 is configured 440 to record information regarding the at least one sensor 400. In at least one embodiment, the at least one memory location 430 is configured 450 to record information regarding at least one of a sensed condition, history, or performance of the device. In at least one embodiment, the at least one memory location 430 is configured 460 to record information regarding at least one of the date, time, quantity of material delivered, presence of one or more biological signaling molecules, or level of one or more biological signaling molecules. In at least one embodiment, the device further includes at least one information transmission mechanism 470 configured to transmit information recorded by the at least one electronic memory location. In at least one embodiment, the device further includes a time-release regulator 480 for the release over time of the at least one therapeutic composition (220, 221, 222, or 223). In at least one embodiment, the device includes at least one receiver configured to obtain release instructions or authorization to release the at least one therapeutic composition 490.

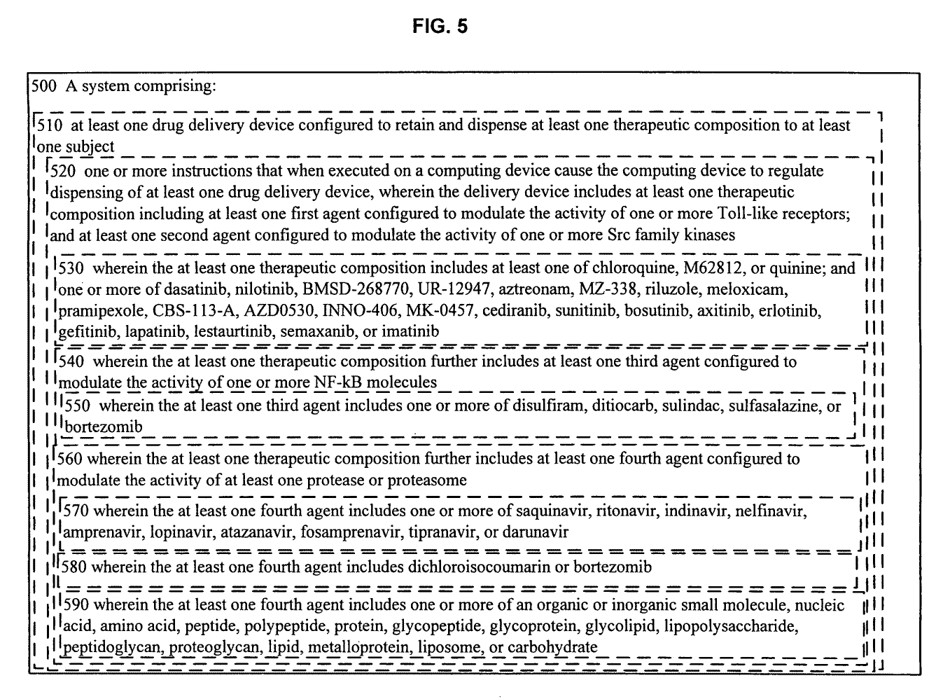

As indicated in FIG. 5, a system 500 is illustrated including at least one drug delivery device 510 configured to retain and dispense at least one therapeutic composition to at least one subject. In an embodiment, the system includes one or more instructions 520 that when executed on a computing device cause the computing device to regulate dispensing of at least one drug delivery device, wherein the delivery device includes at least one therapeutic composition including at least one first agent configured to modulate the activity of one or more Toll-like receptors; and at least one second agent configured to modulate the activity of one or more Src family kinases.

In an embodiment, the at least one therapeutic composition includes at least one of chloroquine, M62812, or quinine; and one or more of dasatinib, nilotinib, BMSD-268770, UR-12947, aztreonam, MZ-338, riluzole, meloxicam, pramipexole, CBS-113-A, AXD0530, INNO-406, MK-0457, cediranib, sunitinib, bosutinib, axitinib, erlotinib, gefitinib, lapatinib, lestaurtinib, semaxanib, or imatinib 530. In an embodiment, the at least one therapeutic composition further includes at least one third agent configured to modulate the activity of one or more NF-kB molecules 540. In an embodiment, the at least one third agent includes one or more of disulfiram, ditiocarb, sulindac, sulfasalazine, or bortezomib 550. In at least one embodiment, the at least one therapeutic composition further includes at least one fourth agent configured to modulate the activity of at least one protease or proteasome 560. In an embodiment, the at least one fourth agent includes one or more of saquinavir, ritonavir, indinavir, nelfinavir, amprenavir, lopinavir, atazanavir, fosamprenavir, tipranavir, or darunavir 570. In an embodiment the at least one fourth agent includes dichloroisocoumarin or bortezomib 580. In at least one embodiment, the at least one fourth agent includes one or more of an organic or inorganic small molecule, nucleic acid, amino acid, peptide, polypeptide, protein, glycopeptide, glycoprotein, glycolipid, lipopolysaccharide, peptidoglycan, proteoglycan, lipid, metalloprotein, liposome, or carbohydrate 590.

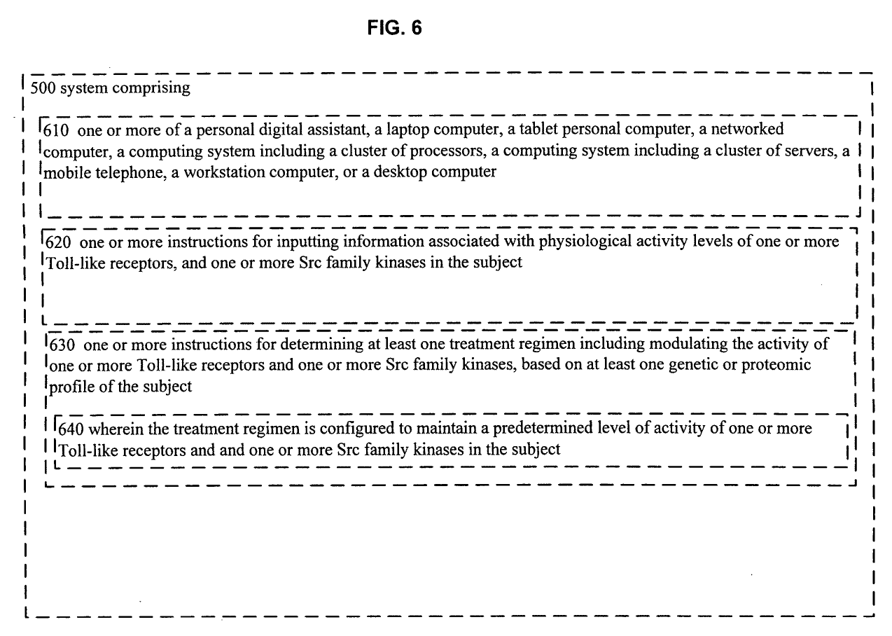

As indicated in FIG. 6, in an embodiment, the system 500 includes one or more computing device 530 including a personal digital assistant (PDA), laptop computer, tablet personal computer, networked computer, computing system including a cluster of processors, computing system including a cluster of servers, mobile telephone, workstation computer, or desktop computer 610. In at least one embodiment, the system includes one or more instructions 620 for inputting information associated with physiological activity levels of one or more Toll-like receptors, and one or more Src family kinases in the subject. In an embodiment, the system includes one or more instructions for determining at least one treatment regimen including modulating the activity of one or more Toll-like receptors, and one or more Src family kinases, based on at least one genetic or proteomic profile of the subject 630. In at least one embodiment, the treatment regimen is configured 640 to maintain a predetermined level of activity of one or more Toll-like receptors, and one or more Src family kinases in the subject.

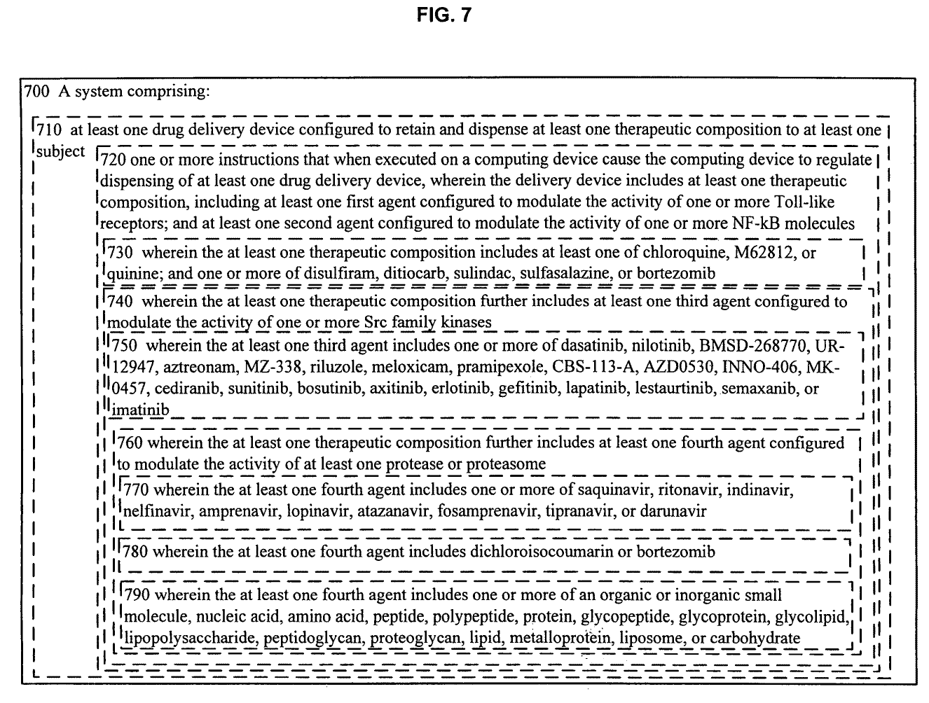

As indicated in FIG. 7, an embodiment of a system 700 includes at least one drug delivery device 710 configured to retain and dispense at least one therapeutic composition to at least one subject. In an embodiment, the system includes one or more instructions 720 that when executed on a computing device cause the computing device to regulate dispensing of at least one drug delivery device, wherein the delivery device includes at least one therapeutic composition, including at least one first agent configured to modulate the activity of one or more Toll-like receptors; and at least one second agent configured to modulate the activity of one or more NF-kB molecules.

In at least one embodiment, the therapeutic composition includes at least one of chloroquine, M62812, or quinine; and one or more of disulfiram, ditiocarb, sulindac, sulfasalazine, or bortezomib 730. In at least one embodiment, the at least one therapeutic composition includes at least one third agent configured to modulate the activity of one or more Src family kinases 740. In at least one embodiment, the at least one third agent includes one or more of dasatinib, nilotinib, BMSD-268770, UR-12947, aztreonam, MZ-338, riluzole, meloxicam, pramipexole, CBS-113-A, AZD0530, INNO-406, MK-0457, cediranib, sunitinib, bosutinib, axitinib, erlotinib, gefitinib, lapatinib, lestaurtinib, semaxanib, or imatinib 750.

In at least one embodiment, the at least one therapeutic composition further includes at least one fourth agent configured to modulate the activity of at least one protease or proteasome 760. In an embodiment, the at least one fourth agent includes one or more of saquinavir, ritonavir, indinavir, nelfinavir, amprenavir, lopinavir, atazanavir, fosamprenavir, tipranavir, or darunavir 770. In an embodiment the at least one fourth agent includes dichloroisocoumarin or bortezomib 780. In at least one embodiment, the at least one fourth agent includes one or more of an organic or inorganic small molecule, nucleic acid, amino acid, peptide, polypeptide, protein, glycopeptide, glycoprotein, glycolipid, lipopolysaccharide, peptidoglycan, proteoglycan, lipid, metalloprotein, liposome, or carbohydrate 790.

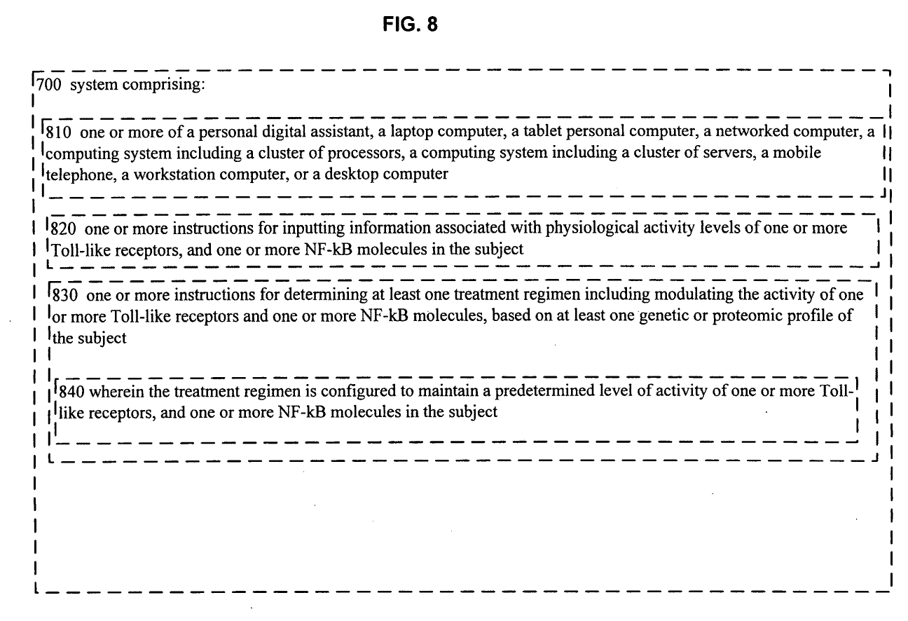

As indicated in FIG. 8, in at least one embodiment, the system includes one or more computing device 810 including a personal digital assistant (PDA), laptop computer, tablet personal computer, networked computer, computing system including a cluster of processors, computing system including a cluster of servers, mobile telephone, workstation computer, or desktop computer. In at least one embodiment, the system includes one or more instructions 820 for inputting information associated with physiological activity levels of one or more Toll-like receptors, and one or more NF-kB molecules in the subject. In at least one embodiment, the system includes one or more instructions 830 for determining at least one treatment regimen including modulating the activity of one or more NF-kB molecules, and one or more Src family kinases, based on at least one genetic or proteomic profile of the subject. In at least one embodiment, the treatment regimen is configured to maintain a predetermined level of activity of one or more NF-kB molecules, and one or more Src family kinases in the subject 840.

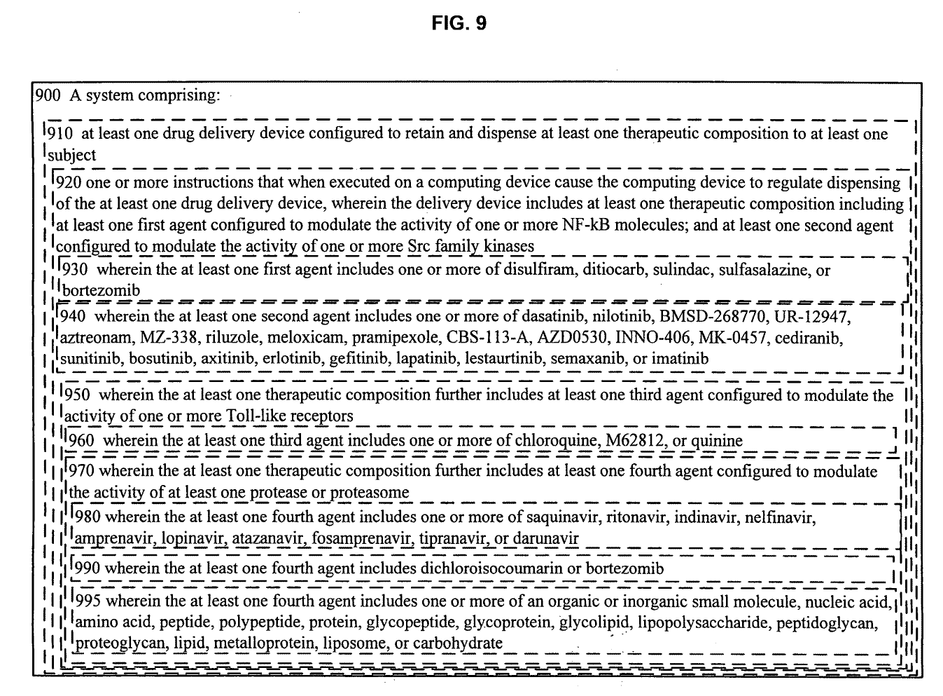

As indicated in FIG. 9, a system 900 is illustrated including at least one drug delivery device 910 configured to retain and dispense at least one therapeutic composition to at least one subject. In at least one embodiment, a system includes one or more instructions 920 that when executed on a computing device cause the computing device to regulate dispensing of the at least one drug delivery device, wherein the delivery device includes at least one therapeutic composition including at least one first agent configured to modulate the activity of one or more NF-kB molecules; and at least one second agent configured to modulate the activity of one or more Src family kinases.

In at least one embodiment, the at least one first agent includes one or more of disulfiram, ditiocarb, sulindac, sulfasalzine, or bortezomib 930. In at least one embodiment, the at least one second agent includes one or more of dasatinib, nilotinib, BMSD-268770, UR-12947, aztreonam, Mz-338, riluzole, meloxicam, pramipexole, CBS-113-A, AZD0530, INN0-406, MK-0457, cediranib, sunitinib, bosutinib, axitinib, erlotinib, gefitinib, lapatinib, lestaurtinib, semaxanib, or imatinib. In at least one embodiment, the at least one therapeutic composition further includes at least one third agent includes at least one third agent configured to modulate the activity of one or more Toll-like receptors 950. In at least one embodiment, the at least one third agent includes one or more of chloroquine, M62812, or quinine 960.

In at least one embodiment, the at least one therapeutic composition further includes at least one fourth agent configured to modulate the activity of at least one protease or proteasome 970. In an embodiment, the at least one fourth agent includes one or more of saquinavir, ritonavir, indinavir, nelfinavir, amprenavir, lopinavir, atazanavir, fosamprenavir, tipranavir, or darunavir 980. In an embodiment the at least one fourth agent includes dichloroisocoumarin or bortezomib 990. In at least one embodiment, the at least one fourth agent includes one or more of an organic or inorganic small molecule, nucleic acid, amino acid, peptide, polypeptide, protein, glycopeptide, glycoprotein, glycolipid, lipopolysaccharide, peptidoglycan, proteoglycan, lipid, metalloprotein, liposome, or carbohydrate 995.

In at least one embodiment, the system includes one or more computing device 1010 including a personal digital assistant (PDA), laptop computer, tablet personal computer, networked computer, computing system including a cluster of processors, computing system including a cluster of servers, mobile telephone, workstation computer, or desktop computer. In at least one embodiment, the system includes one or more instructions 1020 for determining at least one treatment regimen including modulating the activity of one or more NF-kB molecules, and one or more Src family kinases, based on at least one genetic or proteomic profile of the subject. In at least one embodiment, the treatment regimen 1030 is configured to maintain a predetermined level of activity of one or more NF-kB molecules, and one or more Src family kinases in the subject. In at least one embodiment, the system further includes one or more instructions 1040 for inputting information associated with physiological activity levels of one or more NF-kB molecules, and one or more Src family kinases in the subject.

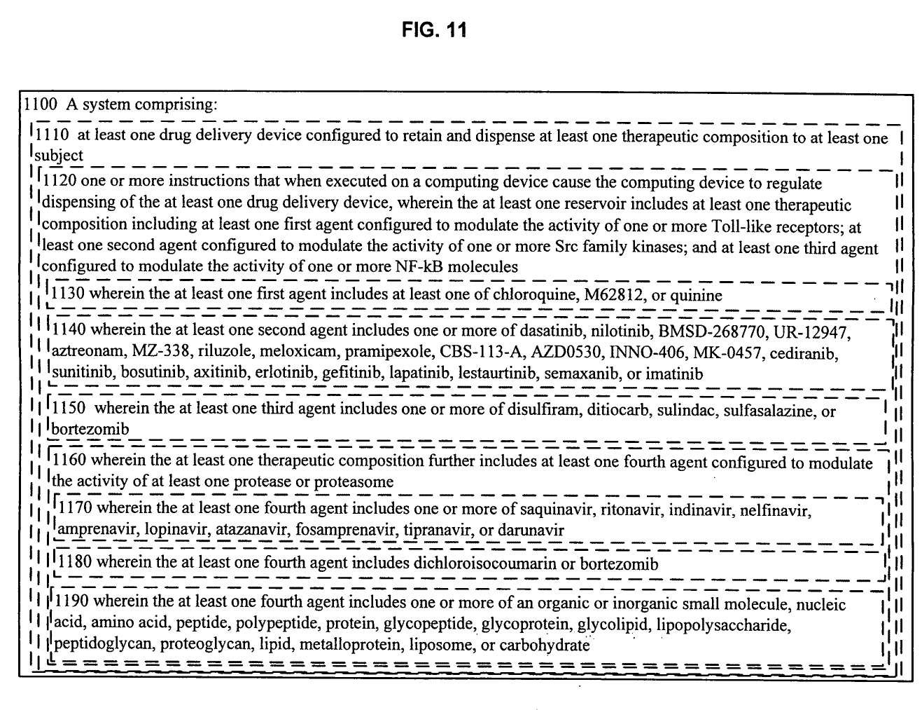

As indicated in FIG. 11, a system 1100 is illustrated including at least one drug delivery device 1110 configured to retain and dispense at least one therapeutic composition to at least one subject. In at least one embodiment, a system includes one or more instructions 1120 that when executed on a computing device cause the computing device to regulate dispensing of the at least one drug delivery device, wherein the delivery device includes at least one therapeutic composition including at least one first agent configured to modulate the activity of one or more Toll-like receptors; at least one second agent configured to modulate the activity of one or more Src family kinases; and at least one third agent configured to modulate the activity of one or more NF-kB molecules.

In at least one embodiment, the at least one first agent includes one or more of chloroquine, M62812, or quinine 1140. In at least one embodiment, the at least one second agent includes one or more of dasatinib, nilotinib, BMSD-268770, UR-12947, aztreonam, MZ-338, riluzole, meloxicam, pramipexole, CBS-113-A, AZD0530, INNO-406, MK-0457, cediranib, sunitinib, bosutinib, axitinib, erlotinib, gefitinib, lapatinib, lestaurtinib, semaxanib, or imatinib 1130.

In at least one embodiment, the at least one therapeutic composition further includes at least one third agent includes one or more of disulfiram, ditiocarb, sulindac, sulfasalazine, or bortezomib 1150. In at least one embodiment, the the at least one therapeutic composition further includes at least one fourth agent configured to modulate the activity of at least one protease or proteasome 1160. In an embodiment the at least one fourth agent includes one or more of squinavir, ritonavir, indinavir, nelfinavir, amprenavir, lopinavir, atazanavir, fosamprenavir, tipranavir, or darunavir 1170.

In at least one embodiment, the at least one fourth agent includes dichloroisocoumarin or bortezomib 1180. In at least one embodiment, the at least one fourth agent includes one or more of an organic or inorganic small molecule, nucleic acid, amino acid, peptide, polypeptide, protein, glycopeptide, glycoprotein, glycolipid, lipopolysaccharide, peptidoglycan, proteoglycan, lipid, metalloprotein, liposome, or carbohydrate 1190.

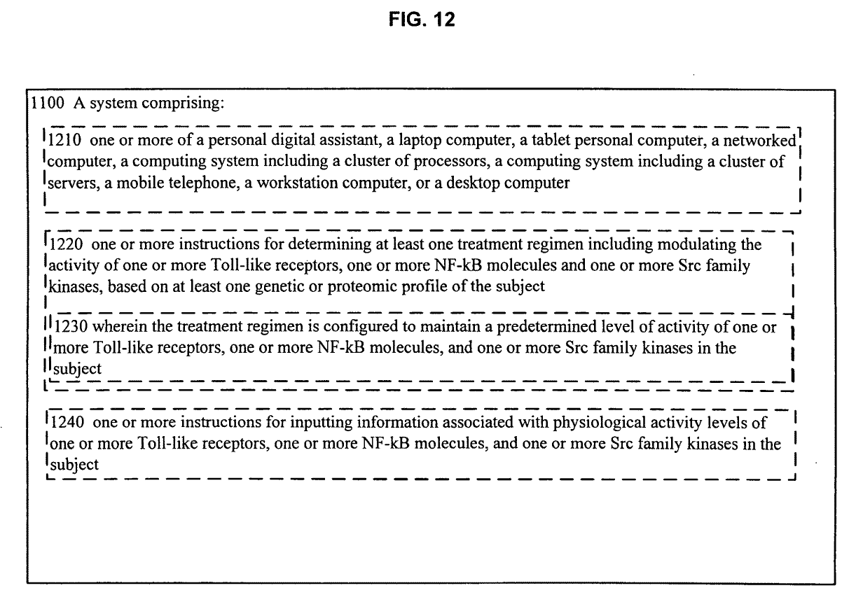

As indicated in FIG. 12, in at least one embodiment, the system includes one or more computing device 1210 including a personal digital assistant (PDA), laptop computer, tablet personal computer, networked computer, computing system including a cluster of processors, computing system including a cluster of servers, mobile telephone, workstation computer, or desktop computer. In at least one embodiment, the system includes one or more instructions 1220 for determining at least one treatment regimen including modulating the activity of one or more Toll-like receptors, one or more NF-kB molecules, and one or more Src family kinases, based on at least one genetic or proteomic profile of the subject. In at least one embodiment, the treatment regimen 1230 is configured to maintain a predetermined level of activity of one or more Toll-like receptors, one or more NF-kB molecules, and one or more Src family kinases in the subject. In at least one embodiment, the system further includes one or more instructions 1240 for inputting information associated with physiological activity levels of one or more Toll-like receptors, one or more NF-kB molecules, and one or more Src family kinases in the subject.

The methods and therapeutic compositions are further described with reference to the following examples; however it is to be understood that the methods and compositions are not limited to such examples.

### EXAMPLES

### Example 1

### Composition Comprising Quinine Sulfate and Dasatinib

An oral therapeutic composition for treatment of malaria, viral infections, bacterial infections, other parasitic infections, sepsis, systemic inflammatory response syndrome, septic shock, multiple organ dysfunction syndrome, autoimmune disease, allergy, cancer, or other inflammatory reactions is prepared containing a first agent that modulates the activity of one or more Toll-like receptors and a second agent that modulates the activity of one or more Src family kinases. The first agent is quinine sulfate (cinchonan-9-ol, 6′-methoxy-, (8.alpha.,9R)-, sulfate (2:1) (salt); C₂₀H₂₄N₂O₂)₂. H₂SO₄.2H₂O); molecular weight 782.96), a modulator of Toll-like receptor 9 activity. The second agent is dasatinib (N-(2-chloro-6-methylphenyl)-2-[[6-[4-(2-hydroxyethyl)-1-piperazinyl]-2-methyl-4-pyrimidinyl]amino]-5-thiazolecarboxamide, monohydrate; C₂₂H₂₆ClN₇O₂S H₂O; molecular mass of 488.01 g/mol), a modulator of Src family kinase activity (particularly of Hck and Lyn). A composition containing quinine sulfate and dasatinib is formulated for oral administration. The therapeutic composition is formulated to enable sufficient dissolution and absorption of the first and second agent to achieve adequate oral bioavailability and systemic dosing.

The oral solid dosage form constitutes one or more tablets. Alternatively, the oral solid dosage form constitutes one or more of a hard or soft gelatin capsule. The oral solid dosage form is taken by a subject or administered to a subject on a periodic basis. For example, tablets or capsules containing quinine sulfate and dasatinib may be administered at least once daily, over the course of about 8 to about 10 days, for example, to treat malaria and other inflammatory reactions. The treatment course can depend on a number of factors, including, for example, severity of the disease or condition and overall patient health. The treatment course or regimen can include from about 1 day to about 28 days; from about 1 day to about 21 days; from about 1 day to about 14 days; from about 1 day to about 7 days; from about 3 days to about 28 days; from about 3 to about 21 days; from about 3 to about 14 days; from about 3 to about 7 days; from about 5 to about 28 days; from about 5 to about 21 days; from about 5 to about 14 days; from about 5 to about 7 days; or any length of time therebetween or greater.

Each dose for an adult of the composition containing quinine sulfate and dasatinib would include about 648 mg of quinine sulfate and about 70 mg of dasatinib. Dosing of the composition may be once every 12 hours, for example. Alternatively, it may be beneficial to administer the combination of quinine sulfate and dasatinib as two or more tablets or capsules, two or more times per day over the course of treatment. In this instance, each tablet may contain about 324 mg of quinine sulfate and about 35 mg of dasatinib. Tablets containing a smaller dose of quinine sulfate and dasatinib may be useful for treating less severe disease or smaller subjects such as, for example, pediatric subjects. For example, quinine sulfate has been administered as a single agent at 10 mg/kg in the pediatric population. Similarly, dasatinib has been administered as a single agent in the pediatric population at doses ranging from 60 to 160 mg/m² (or approximately 2-5 mg/kg) (See, e.g., Porkka, et al., Blood Vol. 112, pp. 1005-1012 (2008) which is herein incorporated by reference). As such, the combination oral dosage form intended for administration at least once daily may contain an amount of quinine sulfate ranging from about 10 mg to about 1296 mg and an amount of dasatinib ranging from about 10 mg to about 140 mg. Tablets containing larger doses of quinine sulfate, dasatinib, or both may also be generated.

The single oral dosage form containing quinine sulfate and dasatinib may also include a number of inactive ingredients or excipients. For example, the tablets may include excipients that are one or more of fillers, binders, lubricants, disintegrants, or combinations thereof. In some instances, a single excipient may have multiple functionalities in the formulation. Fillers are used primarily to create a pill volume that is sufficiently large enough for human fingers to readily handle. Common examples of fillers include lactose, microcrystalline cellulose, corn starch, and sugars such as mannitol, sorbitol, fructose, and dextrose. Binders are used to impart cohesiveness to the tablet formulation that ensures the tablet remains intact after compression. Common examples of binders include starch, gelatin, sugars, and natural and synthetic gums such as acacia and methylcellulose. Lubricants also aide in tablet compression and further prevent the tablets from adhering to the walls of the tablet forming molds. Common examples of lubricants include magnesium stearate, stearic acid, talc, sodium stearyl fumarate and hydrogenated vegetable oil. Polyethylene glycol may also be used to ease tablet removal from the molds. Disintegrants facilitate the dissolution of the tablet in the gastrointestinal tract. Common examples of disintegrants include crospovidone, croscarmellose sodium, and gellan gum. As such, quinine sulfate and dasatinib are formulated in tablet form and may include one or more of the following inactive ingredients: lactose monohydrate, microcrystalline cellulose, croscarmellose sodium, hydroxypropyl cellulose, corn starch, magnesium stearate and talc.

The single oral dosage form containing quinine sulfate and dasatinib may also include a coating that prevents the tablet from dissolving prematurely and may mask an objectionable taste and or smell of the active ingredients. Quinine sulfate in particular has a distinctive bitter taste. As such, tablets containing quinine sulfate and dasatinib are further coated with hypromellose, titanium dioxide, and polyethylene glycol with optional color additives of red and or yellow iron oxides.

In general, the inactive ingredients or excipients included in the single oral dosage form of quinine sulfate and dasatinib and other drug dosing combinations described here are approved for use in human subjects by the Food and Drug Administration (FDA) and are listed in either the United States Pharmacopeia (USP) or National Formulary (NF) for products sold in the United States, or the European Pharmacopeia (EP) for products sold in Europe.

The oral therapeutic composition containing quinine sulfate and dasatinib can be formulated for delayed release. Delayed release permits repetitive, intermittent dosing of the composition from one or more immediate-release units incorporated into a dosage form, for example, repeat-action tablets or capsules. One example includes multilayer or multi-component tablets, caplets or capsules in which each layer or component dissolves or disintegrates to release one or more component of the therapeutic composition. Alternatively, delayed release can include utilizing an enteric delayed release system in which the therapeutic composition is coated with one or more pH sensitive polymer that remains intact in the acidic environment of the stomach and then solubilizes or disintegrates in the more alkaline environment of the small intestine. Polymers used for this purpose include, for example, cellulose acetate phthalate, polyvinylacetate phthalate, hydroxypropylmethylcellulose phthalate, methacrylic acid-methacrylic acid ester copolymers, cellulose acetate trimellitate, carboxymethyl ethylcellulose, or hydroxypropyl methylcellulose acetate succinate.

Alternatively, the oral therapeutic composition containing quinine sulfate and dasatinib can be formulated for extended release to maintain therapeutic blood or tissue levels of the therapeutic composition for a prolonged period of time. Extended release formulations include, for example, diffusion systems, dissolution systems, osmotic systems, mechanical systems, swelling systems, erosion controlled systems, and/or stimulated controlled release systems. A diffusion formulation system may include, for example, reservoir devices in which the oral therapeutic composition is encapsulated by a membrane barrier coat composed, for example, of one or more of hardened gelatin, methyl- or ethylcellulose, polyhydroxymethyacrylate, hydroxypropylcellulose, polyvinylacetate, and/or various waxes.

Alternatively, the diffusion formulation system may include matrix devices in which the oral therapeutic composition is uniformly dissolved or dispersed in an inert polymeric matrix composed, for example, of one or more plastic polymers (e.g., methyl acrylate-methyl methacrylate, polyvinyl chloride, or polyethylene); one or more hydrophilic polymers (e.g., methylcellulose, hydroxypropylmethylcellulose, sodium carboxymethylcellulose, or carbopol 934); one or more fatty compounds (e.g., carnauba wax or glyceryl tristearate), or both. The release rate of the therapeutic composition in a diffusion system is dependent upon the diffusion rate of the therapeutic composition in a diffusion system is dependent upon the diffusion rate of the therapeutic composition through the membrane barrier coat or polymeric matrix. A dissolution system can include, for example, similar formulation excipients, but in this instance the release rate of the therapeutic composition is dependent upon dissolution of the formulation, the therapeutic composition, or both. The dissolution rate can be controlled, for example, by one or more of adjusting the size of encapsulsted drug particles, thickness of coating materials, or diffusivity of core materials.

### Example 2

### Composition Comprising Chloroquine Phosphate and Imatinib

An intravenous therapeutic composition for treatment of malaria, other infections, sepsis, systemic inflammatory response syndrome, septic shock, multiple organ dysfunction syndrome, allergy, or other inflammatory reactions is generated containing a first agent that modulates the activity of one or more Toll-like receptors and a second agent that modulates the activity of one or more Src family kinases. The first agent is chloroquine phosphate (7-chloro-4-[[4-(diethylamino)-1-methylbutyl]amino]quinoline phosphate (1:2); C₁₈H₂₆ClN₃.2H₃PO₄; molecular weight 515.86), a modulator of Toll-like receptor activity. The second agent is imatinib (4-[(4-methyl-1-piperazinyl)methyl]-N-[4-methyl-3-[[4-(3-pyridinyl)-2-pyrimidinyl]amino]-phenyl]benzamide methanesulfonate; C₂₉H₃₁N₇O.CH₄SO₃; molecular mass of 589.7 g/mol), a modulator of Src family kinase activity. The composition containing chloroquine phosphate and imatinib is formulated for intravenous administration. Both compounds are soluble in aqueous solution and as such are readily formulated for intravenous administration.

In some instances, the aqueous solution containing chloroquine phosphate and imatinib is sterilized and directly apportioned into injection vials. The aqueous solution is ready for immediate use. Alternatively, the aqueous solution containing chloroquine phosphate and imatinib is freeze-dried directly into injection vials. The freeze-dried powder is reconstituted prior to intravenous injection or infusion. One or more injection vial containing chloroquine phosphate and imatinib may be used over the course of infusion treatment.

Each injection vial of the intravenous dosage form composition containing chloroquine phosphate and imatinib includes at least one dose for a 70 kilogram adult of about 1400 mg of chloroquine phosphate and about 800 mg of imatinib.

Alternative dosage forms may include the same relative amounts of chloroquine phosphate and imatinib, but in smaller quanities. For example, the dosage form may contain chloroquine phosphate and imatinib in amounts of about 700 mg/400 mg, about 350 mg/200 mg, about 175 mg/100 mg, etc., respectively. Alternative dosage forms may be generated to include different relative amounts of chloroquine phosphate and imatinib. Alternative dosage forms may be determined empirically.

The intravenous dosage form composition containing chloroquine phosphate and imatinib may include additional inactive ingredients or excipients such as, for example, antimicrobial agents, buffers, antioxidants, tonicity agents, and or cryoprotectants and lyoprotectants. Antimicrobial agents in bacteriostatic or fungistatic concentrations may be added to preparations of multiple dose preparations to prevent possible microbial growth inadvertently introduced during withdrawal of a portion of the vial contents. Common examples of antimicrobial agents include phenylmercuric nitrate, thimerosal, benzethonium chloride, benzalkonium chloride, phenol, cresol and or chlorobutanol. Buffers are used to stabilize a solution against chemical or physical degradation. Common acid salts used as buffers include citrates, acetates and phosphates. Antioxidants are used to preserve products against oxidation. Common examples of antioxidants include sodium bisulfite, ascorbic acid, and salts thereof. Tonicity agents are used to ensure that injected material is isotonic with physiological fluids. Common examples of tonicity agents include electrolytes and monosaccharides or disaccharides. Cryoprotectants and lyoprotectants are additives that protect active ingredients from damage due to the freeze-drying process. Common cryoprotectant and lyoprotectant agents include sugars, amino acids, polymers, and polyols. As such, the single intravenous dosing form of chloroquine phosphate and imatinib may include one or more of these inactive ingredients, depending upon whether the dosing form is a solution or a freeze-dried powder.

For use of the freeze-dried powder, the powder is reconstituted in an appropriate aqueous vehicle prior to initiating intravenous administration. An appropriate aqueous vehicle can be highly purified and sterile water or Water for Injection (WFI). The latter is prepared by distillation or by membrane technologies such as reverse osmosis or ultrafiltration. Alternatively, the freeze dried power is reconstituted with a physiologically appropriate vehicle such as sodium chloride or saline solution (0.9%), Ringer's solution, dextrose solution, lactated Ringer's solution, or dextrose and saline solution. The reconstituted solution of chloroquine phosphate and imatinib is infused over the course of several hours using an infusion pump. Alternatively, the reconstituted chloroquine phosphate and imatinib are infused over the course of several hours by addition to an intravenous fluid bag. By way of example, chloroquine phosphate as a single agent has been reportedly infused at 400 mg over one hour without complication (See e.g., Looareesuwan, et al., Br. J. Clin. Pharmac. Vol. 22, pp. 31-36 (1986), which is herein incorporated by reference).

In some instances, flexibility in the dosing of chloroquine phosphate and imatinib may be needed to treat a subject with malaria, other infections, sepsis, systemic inflammatory response syndrome, septic shock, multiple organ dysfunction, or other inflammatory reactions. For example, the appropriate dose of chloroquine phosphate and/or imatinib may be dependent upon one or more characteristic of the subject such as, for example, body weight (kilogram, kg), body surface area (meters squared, m²), gender, age, overall health status and severity of disease. For example, the recommended intravenous dose of chloroquine phosphate ranges from about 10 to about 20 mg/kg in a 24 hour period. As such, only a portion of an intravenous dosage form containing about 1400 mg of chloroquine phosphate and about 800 mg of imatinib, for example, may be administered by infusion over a 24 hour period, depending upon the one or more characteristic of the subject. The intravenous dose composition containing chloroquine phosphate and imatinib may be administered using an infusion pump or an intravenous fluid bag filled with a physiological solution such as standard saline solution.

The composition containing chloroquine phosphate and imatinib may be administered by other parenteral dosing routes such as, for example, intramuscular or subcutaneous injection using, for example, the above-referenced dosages and formulations.

### Example 3

### Composition Comprising Quinine Sulfate, Dasatinib, and Nilotinib

An oral therapeutic composition for treatment of malaria, sepsis, systemic inflammatory response syndrome, septic shock, multiple organ dysfunction syndrome, other infections, allergy, autoimmune disease, or other inflammatory reactions is generated containing a first agent that modulates the activity of one or more Toll-like receptors and two second agents that modulate the activity of one or more Src family kinases. The first agent is quinine sulfate (cinchonan-9-ol, 6′-methoxy-, (8.alpha.,9R)-, sulfate (2:1) (salt); C₂₀H₂₄N₂O₂)₂.H₂SO₄.2H₂O); molecular weight 782.96), a modulator of Toll-like receptor activity. The two second agents are dasatinib (N-(2-chloro-6-methylphenyl)-2-[[6-[4-(2-hydroxyethyl)-1-piperazinyl]-2-methyl-4-pyrimidinyl]amino]-5-thiazolecarboxamide, monohydrate; C₂₂H₂₆ClN₇O₂S.H₂O; molecular mass of 488.01 g/mol) and nilotinib (4-methyl-N-[3-(4-methyl-1H-imidazol-1-yl)-5-(trifluoromethyl)phenyl]-3-[[4-(3-pyridinyl)-2-pyrimidinyl]amino]-benzamide, monohydrochloride, monohydrate; C₂₈H₂₂F₃N₇O HCl.H₂O; molecular mass of 565.98 gm/mol), modulators of Src family kinase activity. A composition containing quinine sulfate, dasatinib and nilotinib is formulated for oral administration. The therapeutic composition is formulated to enable sufficient dissolution and absorption of the first and second agents to achieve adequate oral bioavailability and systemic dosing.

The therapeutic composition contains a first and two second agents that constitute the active ingredients of the therapeutic composition. The active ingredients quinine sulfate, dasatinib, and nilotinib, for example, are combined in a single oral solid dosage form for oral administration. The oral solid dosage form constitutes one or more tablets. Alternatively the oral solid dosage form constitutes one or more of a hard or soft gelatin capsule. The oral solid dosage form is taken by a subject or administered to a subject on a periodic basis. For example, tablets containing quinine sulfate, dasatinib, and nilotinib may be administered at least once daily, over the course of about 8 to about 10 days, for example, to treat malaria and other inflammatory reactions. The treatment course or regimen can include from about 1 day to about 28 days; from about 1 day to about 21 days; from about 1 day to about 14 days; from about 1 day to about 7 days; from about 3 days to about 28 days; from about 3 to about 21 days; from about 3 to about 14 days; from about 3 to about 7 days; from about 5 to about 28 days; from about 5 to about 21 days; from about 5 to about 14 days; from about 5 to about 7 days; or any length of time therebetween or greater.

Each dose of the composition containing quinine sulfate, dasatinib, and nilotinib formulated for an adult would include about 648 mg of quinine sulfate, about 70 mg of dasatinib, and about 400 mg of nilotinib and be administered about every 12 hours, for example. Alternatively, it may be beneficial to administer the combination of quinine sulfate, dasatini, and nilotinib as two or more tablets, two or more times per day over the course of about 8 to about 10 days, for example. In this instance, each tablet contains about 324 mg of quinine sulfate, about 35 mg of dasatinib, and about 200 mg of nilotinib. The treatment course or regimen can include from about 1 day to about 28 days; from about 1 day to about 21 days; from about 1 day to about 14 days; from about 1 day to about 7 days; from about 3 days to about 28 days; from about 3 to about 21 days; from about 3 to about 14 days; from about 3 to about 7 days; from about 5 to about 28 days; from about 5 to about 21 days; from about 5 to about 14 days; from about 5 to about 7 days; or any length of time therebetween or greater.

Dosage forms containing more or less of each compound may also be contemplated for use in more or less severe disease or in the pediatric population, for example. As such, the combination oral dosage form intended for administration at least once daily may contain an amount of quinine sulfate ranging from about 10 mg to about 1296 mg, an amount of dasatinib ranging from about 10 mg to about 140 mg, and an amount of nilotinib ranging from about 10 to about 800 mg. Tablets containing larger doses of quinine sulfate, dasatinib, and/or nilotinib may also be generated. Alternatively, the amount of quinine sulfate, dasatinib, and nilotinib in the composition may be determined empirically.

The oral dosage form containing quinine sulfate, dasatinib and nilotinib may also include a number of inactive ingredients or excipients, examples of which have been described herein. As such, quinine sulfate, dasatinib, and nilotinib are formulated in tablet form and may include one or more of the following inactive ingredients: lactose monohydrate, microcrystalline cellulose, colloidal silicon dioxide, crospovidone, polyoxamer 188, croscarmellose sodium, hydroxypropyl cellulose, corn starch, magnesium stearate and talc.

The oral dosage form containing quinine sulfate and dasatinib may also include a coating that prevents the tablet from dissolving prematurely and may mask an objectionable taste and or smell of the active ingredients. Quinine in particular has a distinctive bitter taste. As such, tablets containing quinine sulfate and dasatinib may be further coated with one or more of the following inactive coating ingredients: gelatin, hypromellose, titanium dioxide, and polyethylene glycol with optional color additives of red and or yellow iron oxides.

The oral therapeutic composition containing quinine sulfate, dasatinib, and nilotinib can be formulated for delayed release. Delayed release permits repetitive, intermittent dosing of the composition from one or more immediate-release units incorporated into a dosage form, for example, repeat-action tablets or capsules. One example includes multilayer or multi-component tablets, caplets or capsules in which each layer or component dissolves or disintegrates to release one or more component of the therapeutic composition. Alternatively, delayed release can include utilizing an enteric delayed release system in which the therapeutic composition is coated with one or more pH sensitive polymer that remains intact in the acidic environment of the stomach and then solubilizes or disintegrates in the more alkaline environment of the small intestine. Polymers used for this purpose include, for example, cellulose acetate phthalate, polyvinylacetate phthalate, hydroxypropylmethylcellulose phthalate, methacrylic acid-methacrylic acid ester copolymers, cellulose acetate trimellitate, carboxymethyl ethylcellulose, or hydroxypropyl methylcellulose acetate succinate.

Alternatively, the oral therapeutic composition containing quinine sulfate, dasatinib, and nilotinib can be formulated for extended release to maintain therapeutic blood or tissue levels of the therapeutic composition for a prolonged period of time. Extended release formulations include, for example, diffusion systems, dissolution systems, osmotic systems, mechanical systems, swelling systems, erosion controlled systems, and/or stimulated controlled release systems. A diffusion formulation system may include, for example, reservoir devices in which the oral therapeutic composition is encapsulated by a membrane barrier coat composed, for example, of one or more of hardened gelatin, methyl- or ethylcellulose, polyhydroxymethyacrylate, hydroxypropylcellulose, polyvinylacetate, and/or various waxes.

Alternatively, the diffusion formulation system may include matrix devices in which the oral therapeutic composition is uniformly dissolved or dispersed in an inert polymeric matrix composed, for example, of one or more plastic polymers (e.g., methyl acrylate-methyl methacrylate, polyvinyl chloride, or polyethylene); one or more hydrophilic polymers (e.g., methylcellulose, hydroxypropylmethylcellulose, sodium carboxymethylcellulose, or carbopol 934); one or more fatty compounds (e.g., carnauba wax or glyceryl tristearate), or both. The release rate of the therapeutic composition in a diffusion system is dependent upon the diffusion rate of the therapeutic composition in a diffusion system is dependent upon the diffusion rate of the therapeutic composition through the membrane barrier coat or polymeric matrix. A dissolution system can include, for example, similar formulation excipients, but in this instance the release rate of the therapeutic composition is dependent upon dissolution of the formulation, the therapeutic composition, or both. The dissolution rate can be controlled, for example, by one or more of adjusting the size of encapsulated drug particles, thickness of coating materials, or diffusivity of core materials.

### Example 4

### Composition Comprising Chloroquine Phosphate and Disulfiram

An oral therapeutic composition for treatment of malaria, other infections, sepsis, systemic inflammatory response syndrome, septic shock, multiple organ dysfunction syndrome, allergy, autoimmune disease, cancer, or other inflammatory reactions is generated containing a first agent that modulates the activity of one or more Toll-like receptors and a second agent that modulates the activity one or more NF-kB molecules. The first agent is chloroquine phosphate (7-chloro-4-[[4-(diethylamino)-1-methylbutyl]amino]quinoline phosphate (1:2); C₁₈H₂₆ClN₃.2H₃PO₄; molecular weight 515.86), a modulator of Toll-like receptor activity. The second agent is disulfiram (1-(diethylthiocarbamoyldisulfanyl)-N,N-diethyl-methanethioamide; C₁₀H₂₀N₂S₄; molecular mass of 296.53 gm/mol), a modulator of NF-kB activity. A composition containing chloroquine phosphate and disulfiram is formulated for oral administration. The therapeutic composition is formulated to enable sufficient dissolution and absorption of the first and second agent to achieve adequate oral bioavailability and systemic dosing.

The therapeutic composition contains a first agent and a second agent that constitute the active ingredients of the therapeutic composition. The active ingredients chloroquine phosphate and disulfiram, for example, are combined in a single oral solid dosage form for oral administration. The oral solid dosage form constitutes one or more tablets. Alternatively the oral solid dosage form constitutes one or more of a hard or soft gelatin capsule. The oral solid dosage form is taken by a subject or administered to a subject on a periodic basis. Chloroquine phosphate and disulfiram have reported elimination half-lives in human subjects ranging from about 60 to about 120 hours. As such, chloroquine phosphate and disulfiram may be administered once daily. For example, tablets containing chloroquine phosphate and disulfiram may be administered at least once daily, over the course of about 3 to about 4 days, for example, to treat malaria and other inflammatory reactions.

The treatment course or regimen can include from about 1 day to about 28 days; from about 1 day to about 21 days; from about 1 day to about 14 days; from about 1 day to about 7 days; from about 3 days to about 28 days; from about 3 to about 21 days; from about 3 to about 14 days; from about 3 to about 7 days; from about 5 to about 28 days; from about 5 to about 21 days; from about 5 to about 14 days; from about 5 to about 7 days; or any length of time therebetween or greater.

Each dose of the composition containing chloroquine phosphate and disulfiram formulated for an adult would include about 500 mg of chloroquine phosphate and about 250 mg of disulfiram. At the initiation of treatment, two doses may be given in about the first 24 hours, followed by one dose on each of about two to three consecutive days, for example. Alternatively, it may be beneficial to administer the combination of chloroquine phosphate and disulfiram as two or more tablets, two or more times per day over the treatment period. For example, each tablet may contain about 250 mg of chloroquine phosphate and about 125 mg of disulfiram. Dosage forms containing more or less of each compound may also be contemplated for use in more or less severe disease or in the pediatric population, for example. As such, the combination oral dosage form intended for administration at least once daily may contain an amount of chloroquine phosphate ranging from about 10 mg to about 1000 mg and an amount of disulfiram ranging from about 10 mg to about 500 mg. Tablets containing larger doses of chloroquine phosphate and/or disulfiram may also be contemplated. Alternatively, the amount of chloroquine phosphate and disulfiram in the composition may be determined empirically.

The oral dosage form containing chloroquine phosphate and disulfiram may also include a number of inactive ingredients or excipients. For example, the tablets may include excipients that are one or more of fillers, binders, lubricants, disintegrants, or combinations thereof. In some instances, a single excipient may have multiple functionalities in the formulation. Fillers are used primarily to create a pill volume that is sufficiently large enough for human fingers to readily handle. Common examples of fillers include lactose, microcrystalline cellulose, corn starch, and sugars such as mannitol, sorbitol, fructose, and dextrose. Binders are used to impart cohesiveness to the tablet formulation that ensures the tablet remains intact after compression. Common examples of binders include starch, gelatin, sugars, and natural and synthetic gums such as acacia and methylcellulose. Lubricants also aide in tablet compression and further prevent the tablets from adhering to the walls of the tablet forming molds. Common examples of lubricants include magnesium stearate, stearic acid, sodium stearyl fumarate and hydrogenated vegetable oil. Polyethylene glycol may also be used to allow the tablet to drop more readily out of the mold. Disintegrants facilitate the dissolution of the tablet in the gastrointestinal tract. Common examples of disintegrants include starch, gums, clays, crospovidone, and croscarmellose sodium. As such, chloroquine sulfate and disulfiram are formulated in tablet form and may include one or more of the following inactive ingredients: magnesium aluminum silicate, magnesium stearate, crospovidone, starch, carnauba wax, colloidal silicon dioxide, dibasic calcium phosphate, hydroxypropyl methylcellulose, microcrystalline cellulose, polyethylene glycol, pregelatininzed, polysorbate 80, sodium starch glycolate, stearic acid, and titanium dioxide.

In general, the inactive ingredients or excipients included in the single oral dosage form of chloroquine phosphate and disulfiram and other drug dosing combinations described herein are approved for use in human subjects by the Food and Drug Administration (FDA) and are listed in either the United States Pharmacopeia (USP) or National Formulary (NF) for products sold in the United States, or the European Pharmacopeia (EP) for products sold in Europe.

The oral therapeutic composition containing chloroquine sulfate and disulfiram can be formulated for delayed release. Delayed release permits repetitive, intermittent dosing of the composition from one or more immediate-release units incorporated into a dosage form, for example, repeat-action tablets or capsules. One example includes multilayer or multi-component tablets, caplets or capsules in which each layer or component dissolves or disintegrates to release one or more component of the therapeutic composition. Alternatively, delayed release can include utilizing an enteric delayed release system in which the therapeutic composition is coated with one or more pH sensitive polymer that remains intact in the acidic environment of the stomach and then solubilizes or disintegrates in the more alkaline environment of the small intestine. Polymers used for this purpose include, for example, cellulose acetate phthalate, polyvinylacetate phthalate, hydroxypropylmethylcellulose phthalate, methacrylic acid-methacrylic acid ester copolymers, cellulose acetate trimellitate, carboxymethyl ethylcellulose, or hydroxypropyl methylcellulose acetate succinate.

Alternatively, the oral therapeutic composition containing chloroquine sulfate and disufiram can be formulated for extended release to maintain therapeutic blood or tissue levels of the therapeutic composition for a prolonged period of time. Extended release formulations include, for example, diffusion systems, dissolution systems, osmotic systems, mechanical systems, swelling systems, erosion controlled systems, and/or stimulated controlled release systems. A diffusion formulation system may include, for example, reservoir devices in which the oral therapeutic composition is encapsulated by a membrane barrier coat composed, for example, of one or more of hardened gelatin, methyl- or ethylcellulose, polyhydroxymethyacrylate, hydroxypropylcellulose, polyvinylacetate, and/or various waxes.

Alternatively, the diffusion formulation system may include matrix devices in which the oral therapeutic composition is uniformly dissolved or dispersed in an inert polymeric matrix composed, for example, of one or more plastic polymers (e.g., methyl acrylate-methyl methacrylate, polyvinyl chloride, or polyethylene); one or more hydrophilic polymers (e.g., methylcellulose, hydroxypropylmethylcellulose, sodium carboxymethylcellulose, or carbopol 934); one or more fatty compounds (e.g., carnauba wax or glyceryl tristearate), or both. The release rate of the therapeutic composition in a diffusion system is dependent upon the diffusion rate of the therapeutic composition in a diffusion system is dependent upon the diffusion rate of the therapeutic composition through the membrane barrier coat or polymeric matrix. A dissolution system can include, for example, similar formulation excipients, but in this instance the release rate of the therapeutic composition is dependent upon dissolution of the formulation, the therapeutic composition, or both. The dissolution rate can be controlled, for example, by one or more of adjusting the size of encapsulsted drug particles, thickness of coating materials, or diffusivity of core materials.

### Example 5

### Composition Comprising Quinine Sulfate and Bortezomib

An intravenous therapeutic composition for treatment of malaria, other infections, sepsis, systemic inflammatory response syndrome, septic shock, multiple organ dysfunction syndrome, allergy, cancer, autoimmune disease, or other inflammatory reactions is generated containing a first agent that modulates the activity of one or more Toll-like receptors and a second agent that modulates the activity of one or more NF-kB molecules. The first agent is quinine sulfate (cinchonan-9-ol, 6′-methoxy-, (8.alpha.,9R)-, sulfate (2:1) (salt); C₂₀H₂₄N₂O₂)₂.H₂SO₄.2H₂O); molecular weight 782.96), a modulator of Toll-like receptor activity. The second agent is bortezomib ([(1R)-3-methyl-1-[[(2S)-1-oxo-3-phenyl-2-[(pyrazinylcarbonyl)amino]propyl]amino]butyl]boronic acid; C₁₉H₂₅BN₄O₄; molecular mass of 384.24 gm/mol), a modulator of NF-kB activity, and also a proteasome inhibitor. A composition containing quinine sulfate and bortezomib is formulated for intravenous administration.

The therapeutic composition contains a first and a second agent that constitute the active ingredients of the therapeutic composition. The active ingredients quinine sulfate and bortezomib, for example, are combined in aqueous solution. In some instances, the aqueous solution containing quinine sulfate and bortezomib is sterilized and directly apportioned into injection vials. The aqueous solution is ready for immediate use. Alternatively, the aqueous solution containing quinine sulfate and bortezomib is freeze-dried directly into injection vials. The freeze-dried powder is reconstituted prior to intravenous infusion. One or more injection vial containing quinine sulfate and bortezomib may be used over the course of infusion treatment.

Each injection vial of the intravenous dosage form composition containing quinine sulfate and bortezomib includes at least one dose for a 70 kilogram adult of about 2300 mg of quinine sulfate and about 2.2 mg of bortezomib. Alternative dosage forms may include the same relative amounts of quinine sulfate and bortezomib, but in smaller quanities. For example, the dosage form may contain quinine sulfate and bortezomib in amounts of about 1150 mg/1.1 mg, about 575 mg/0.55 mg, about 230 mg/0.22 mg, etc., respectively. Alternative dosage forms may be generated to include different relative amounts of chloroquine phosphate and imatinib. Alternative dosage forms may be determined empirically.

The treatment course or regimen can include from about 1 day to about 28 days; from about 1 day to about 21 days; from about 1 day to about 14 days; from about 1 day to about 7 days; from about 3 days to about 28 days; from about 3 to about 21 days; from about 3 to about 14 days; from about 3 to about 7 days; from about 5 to about 28 days; from about 5 to about 21 days; from about 5 to about 14 days; from about 5 to about 7 days; or any length of time therebetween or greater.

The intravenous dosage form composition containing quinine sulfate and bortezomib may include additional inactive ingredients or excipients such as, for example, antimicrobial agents, buffers, antioxidants, tonicity agents, and or cryoprotectants and lyoprotectants. Antimicrobial agents in bacteriostatic or fungistatic concentrations may be added to preparations of multiple dose preparations to prevent possible microbial growth inadvertently introduced during withdrawal of a portion of the vial contents. Common examples of antimicrobial agents include phenylmercuric nitrate, thimerosal, benzethonium chloride, benzalkonium chloride, phenol, cresol and or chlorobutanol. Buffers are used to stabilize a solution against chemical or physical degradation. Common acid salts used as buffers include citrates, acetates and phosphates. Antioxidants are used to preserve products against oxidation. Common examples of antioxidants include sodium bisulfite, ascorbic acid, and salts thereof. Tonicity agents are used to ensure that injected material is isotonic with physiological fluids. Common examples of tonicity agents include electrolytes and mono- or disaccharides. Cryoprotectants and lyoprotectants are additives that protect active ingredients from damage due to the freeze-drying process. Common cryoprotectant and lyoprotectant agents include sugars, amino acids, polymers, and polyols. As such, the intravenous dosage form of quinine sulfate and bortezomib may include one or more of these inactive ingredients, depending upon whether the dosing form is a solution or a freeze-dried powder. For example, quinine sulfate and bortezomib in an intravenous dosage form may be prepared with mannitol, a polyol sugar alcohol.

For administration of the freeze-dried powder, the powder is reconstituted in an appropriate aqueous vehicle prior to initiating intravenous administration. An appropriate aqueous vehicle can be highly purified and sterile water or Water for Injection (WFI). The latter is prepared by distillation or by membrane technologies such as reverse osmosis or ultrafiltration. Alternatively, the freeze dried power is reconstituted with a physiologically appropriate vehicle such as sodium chloride or saline solution (0.9%), Ringer's solution, dextrose solution, lactated Ringer's solution, or dextrose and sodium chloride (0.9%) solution. The reconstituted solution of quinine sulfate and bortezomib is infused over the course of several hours using an infusion pump. Alternatively, the reconstituted solution of quinine sulfate and bortezomib is infused over the course of several hours by addition to an intravenous fluid bag.

In some instances, flexibility in the dosing of quinine sulfate and bortezomib may be need to treat a subject with malaria, other infections, sepsis, systemic inflammatory response syndrome, septic shock, multiple organ dysfunction, or other inflammatory reactions. For example, the appropriate dose of quinine sulfate and/or bortezomib may be dependent upon one or more characteristic of the subject such as, for example, body weight (kilogram, kg), body surface area (meters squared, m²), gender, age, overall health status and severity of disease. For example, the recommended intravenous dose of quinine sulfate ranges from about 8.2 to about 16.4 mg/kg in a 24 hour period. The recommended intravenous dose of bortezomib is about 1.3 mg/m² or about 0.03 mg/kg. As such, only a portion of an intravenous dosage form containing about 2300 mg of quinine sulfate and about 2.2 mg of bortezomib, for example, may be administered by infusion over a 24 hour period, depending upon the one or more characteristic of the subject. The intravenous dose composition containing quinine sulfate and bortezomib may be administered using an infusion pump or an intravenous fluid bag filled with a physiological solution such as standard saline solution.

The composition containing quinine sulfate and bortezomib may be administered by other parenteral dosing routes such as, for example, intramuscular or subcutaneous injection using, for example, the above-referenced dosages and formulations.

### Example 6

### Composition Comprising Chloroquine Phosphates Disulfiram, and Bortezomib

An intravenous therapeutic composition for treatment of malaria, other infections, sepsis, systemic inflammatory response syndrome, septic shock, multiple organ dysfunction syndrome, allergy, autoimmune disease, cancer, or other inflammatory reactions is generated containing a first agent that modulates the activity of one or more Toll-like receptors and two second agents that modulate the activity of one or more NF-kB molecules. The first agent is chloroquine phosphate (7-chloro-4-[[4-(diethylamino)-1-methylbutyl]amino]quinoline phosphate (1:2); C₁₈H₂₆ClN₃.2H₃PO₄; molecular weight 515.86), a modulator of Toll-like receptor activity. The two second agents are disulfiram (1-(diethylthiocarbamoyldisulfanyl)-N,N-diethyl-methanethioamide; C₁₀H₂₀N₂S₄; molecular mass of 296.53 gm/mol) and bortezomib ([(1R)-3-methyl-1-[[(2S)-1-oxo-3-phenyl-2-[(pyrazinylcarbonyl)amino]propyl]amino]butyl]boronic acid; C₁₉H₂₅BN₄O₄; molecular mass of 384.24 gm/mol), modulators of NF-kB activity. Bortezomib is also a proteasome inhibitor. A composition containing chloroquine phosphate, disulfiram, and bortezomib is formulated for intravenous administration.

The therapeutic composition contains a first and a second agent that constitute the active ingredients of the therapeutic composition. The active ingredients chloroquine phosphate, disulfiram, and bortezomib, for example, are combined in aqueous solution. In some instances, the aqueous solution containing chloroquine phosphate, disulfiram, and bortezomib is sterilized and directly apportioned into injection vials and ready for immediate use. Alternatively, the aqueous solution containing chloroquine phosphate, disulfiram, and bortezomib is freeze-dried directly into injection vials. The freeze-dried powder is reconstituted prior to intravenous injection or infusion. One or more injection vials containing quinine sulfate and bortezomib may be used over the course of treatment.

Each injection vial of the intravenous dosage form composition containing chloroquine phosphate, disulfiram, and bortezomib includes at least one dose for a 70 kilogram adult of about 1400 mg of chloroquine phosphate, about 500 mg of disulfiram, and about 2.2 mg bortezomib. Alternative dosage forms may include the same relative amounts of chloroquine phosphate, disulfiram, and bortezomib, but in smaller quanities. For example, the dosage form may contain chloroquine phosphate, disulfiram, and bortezomib in amounts of about 700 mg/250 mg/1.1 mg, about 575 mg/125 mg/0.55 mg, about 230 mg/50 mg/0.22 mg, etc., respectively. Alternative dosage forms may be generated to include different relative amounts of chloroquine phosphate, disulfiram, and bortezomib. Alternative dosage forms may be determined empirically.

The intravenous dosing form containing chloroquine phosphate, disulfiram, and bortezomib may also include additional inactive ingredients or excipients such as, for example, antimicrobial agents, buffers, antioxidants, tonicity agents, and or cryoprotectants and lyoprotectants as described herein. For example, the chloroquine phosphate, disulfiram, and bortezomib intravenous dosage form may include mannitol, a sugar alcohol polyol.

For administration of the freeze-dried powder, the powder is reconstituted in an appropriate aqueous vehicle prior to initiating intravenous administration. An appropriate aqueous vehicle can be highly purified and sterile water or Water for Injection (WFI). Alternatively, the freeze dried power is reconstituted with a physiologically appropriate vehicle such as sodium chloride or saline solution (0.9%), Ringer's solution, dextrose solution, lactated Ringer's solution, or dextrose and sodium chloride (0.9%) solution. The reconstituted solution of chloroquine phosphate, disulfiram, and bortezomib is infused over the course of several hours using an infusion pump. Alternatively, the reconstituted solution of chloroquine phosphate, disulfiram, and bortezomib is infused over the course of several hours by addition to an intravenous fluid bag.

In some instances, flexibility in the dosing of chloroquine phosphate, disulfiram, and bortezomib may be need to effectively treat a subject with malaria, other infection, allergy, cancer, autoimmune disease, or other inflammatory reactions. For example, the appropriate dose of chloroquine phosphate, disulfiram, and bortezomib may be dependent upon one or more characteristic of the subject such as, for example, body weight (kilogram, kg), body surface area (meters squared, m²), gender, age, overall health status and severity of disease. For example, the recommended intravenous dose of chloroquine sulfate ranges from about 10 to about 20 mg/kg in a 24 hour period. The recommended intravenous dose of bortezomib is about 1.3 mg/m² or about 0.03 mg/kg. As such, only a portion of an intravenous dosage form containing about 1400 mg of chloroquine phosphate, about 500 mg of disulfiram, and about 2.2 mg of bortezomib, for example, may be administered by infusion over about a 24 hour period, depending upon the one or more characteristic of the subject. The intravenous dose comprising chloroquine phosphate, disulfiram, and bortezomib may be administered using an infusion pump or an intravenous fluid bag filled with a physiological solution such as standard saline solution.

The composition containing chloroquine phosphate, disulfiram, and bortezomib may be administered by other parenteral dosing routes such as, for example, intramuscular or subcutaneous injection using, for example, the above-referenced dosages and formulations.

### Example 7

### Composition Comprising Disulfiram and Dasatinib

An oral therapeutic composition for treatment of malaria, viral infection, bacterial infection, fungal infection, allergic reaction, or other inflammatory reactions is generated containing a first agent that modulates the activity of one or more NF-kB molecules and a second agent that modulates the activity of one or more Src family kinases. The first agent is disulfiram (1-(diethylthiocarbamoyldisulfanyl)-N,N-diethyl-methanethioamide; C₁₀H₂₀N₂S₄; molecular mass of 296.53 gm/mol), a modulator of NF-kB activity. The second agent is dasatinib (N-(2-chloro-6-methylphenyl)-2-[[6-[4-(2-hydroxyethyl)-1-piperazinyl]-2-methyl-4-pyrimidinyl]amino]-5-thiazolecarboxamide, monohydrate; C₂₂H₂₆ClN₇O₂S.H₂O; molecular mass of 488.01 g/mol), a modulator of Src family kinase activity. A composition containing disulfiram and dasatinib is formulated for oral administration. The therapeutic composition is formulated to enable sufficient dissolution and absorption of the first and second agent to achieve adequate oral bioavailability and systemic dosing.

The therapeutic composition contains a first and a second agent that constitute the active ingredients of the therapeutic composition. The active ingredients disulfiram and dasatinib, for example, are combined in a single oral solid dosage form for oral administration. The oral solid dosage form constitutes one or more tablets. Alternatively the oral solid dosage form constitutes one or more of a hard or soft gelatin capsule. The oral solid dosage form is taken by a subject or administered to a subject on a periodic basis. For example, tablets containing disulfiram and dasatinib may be administered at least once daily, over the course of about 3 to about 10 days, for example, to treat malaria, other infections, or other inflammatory reactions.

The treatment course or regimen can include from about 1 day to about 28 days; from about 1 day to about 21 days; from about 1 day to about 14 days; from about 1 day to about 7 days; from about 3 days to about 28 days; from about 3 to about 21 days; from about 3 to about 14 days; from about 3 to about 7 days; from about 5 to about 28 days; from about 5 to about 21 days; from about 5 to about 14 days; from about 5 to about 7 days; or any length of time therebetween or greater.

Each dose of the composition containing disulfiram and dasatinib formulated for an adult would include about 125 mg of disulfiram and about 70 mg of dasatinib and be administered about every 12 hours, for example. In some instances, a larger dose of disulfiram may be of benefit to a subject in which case the tablets may contain about 250 mg of disulfiram with about 70 mg dasatinib and be administered about every 12 hours, for example. Alternatively, it may be beneficial to administer the combination of disulfiram and dasatinib as two or more tablets, two or more times per day over the course of about 3 to about 10 days, for example. In this instance, each tablet may contain about 67.5 or about 125 mg of disulfiram and about 35 mg of dasatinib.

Tablets containing a smaller dose of disulfiram and dasatinib may be useful for treating less severe disease or small subjects such as, for example, pediatric subjects. For example, dasatinib has been administered as a single agent in the pediatric population at doses ranging from about 60 to about 160 mg/M² (or approximately 2-5 mg/kg) (see, e.g., Porkka, et al., Blood Vol.112, pp. 1005-1012 (2008), which is herein incorporated by reference). As such, the combination oral dosage form intended for administration at least once daily may contain an amount of disulfiram ranging from about 10 mg to about 500 mg and an amount of dasatinib ranging from about 10 mg to about 140 mg. Tablets containing larger doses of disulfiram, dasatinib, or both may also be generated. Alternatively, the amount of disulfiram and dasatinib in the composition may be determined empirically.

The oral dosage form containing disulfiram and dasatinib may also include a number of inactive ingredients or excipients. For example, the tablets may include excipients that are one or more of fillers, binders, lubricants, disintegrants, or combinations thereof. In some instances, a single excipient may have multiple functionalities in the formulation. Fillers are used primarily to create a pill volume that is sufficiently large enough for human fingers to readily handle. Common examples of fillers include lactose, microcrystalline cellulose, corn starch, and sugars such as mannitol, sorbitol, fructose, and dextrose. Binders are used to impart cohesiveness to the tablet formulation that ensures the tablet remains intact after compression. Common examples of binders include starch, gelatin, sugars, and natural and synthetic gums such as acacia and methylcellulose. Lubricants also aide in tablet compression and further prevent the tablets from adhering to the walls of the tablet forming molds. Common examples of lubricants include magnesium stearate, stearic acid, sodium stearyl fumarate and hydrogenated vegetable oil. Polyethylene glycol may also be used to allow the tablet to drop more readily out of the mold. Disintegrants facilitate the dissolution of the tablet in the gastrointestinal tract. Common examples of disintegrants include crospovidone, croscarmellose sodium, and gellan gum. As such, disulfiram and dasatinib are formulated in tablet form and may include one or more of the following inactive ingredients: lactose monohydrate, microcrystalline cellulose, croscarmellose sodium, hydroxypropyl cellulose, magnesium aluminum silicate, magnesium stearate, povidone, and starch.

The oral dosage form containing disulfiram and dasatinib may also include a coating that prevents the tablet from dissolving prematurely and may mask any objectionable taste and or smell of the active ingredients. As such, tablets containing disulfiram and dasatinib are further coated with gelatin, titanium dioxide, and polyethylene glycol with optional color additives of red and or yellow iron oxides.

In general, the inactive ingredients or excipients included in the oral dosage form of disulfiram and dasatinib and other drug dosing combinations described herein are approved for use in human subjects by the Food and Drug Administration (FDA) and are listed in either the United States Pharmacopeia (USP) or National Formulary (NF) for products sold in the United States, or the European Pharmacopeia (EP) for products sold in Europe.

The oral therapeutic composition containing disulfiram and dasatinib can be formulated for delayed release. Delayed release permits repetitive, intermittent dosing of the composition from one or more immediate-release units incorporated into a dosage form, for example, repeat-action tablets or capsules. One example includes multilayer or multi-component tablets, caplets or capsules in which each layer or component dissolves or disintegrates to release one or more component of the therapeutic composition. Alternatively, delayed release can include utilizing an enteric delayed release system in which the therapeutic composition is coated with one or more pH sensitive polymer that remains intact in the acidic environment of the stomach and then solubilizes or disintegrates in the more alkaline environment of the small intestine. Polymers used for this purpose include, for example, cellulose acetate phthalate, polyvinylacetate phthalate, hydroxypropylmethylcellulose phthalate, methacrylic acid-methacrylic acid ester copolymers, cellulose acetate trimellitate, carboxymethyl ethylcellulose, or hydroxypropyl methylcellulose acetate succinate.

Alternatively, the oral therapeutic composition containing disufiram and dasatinib can be formulated for extended release to maintain therapeutic blood or tissue levels of the therapeutic composition for a prolonged period of time. Extended release formulations include, for example, diffusion systems, dissolution systems, osmotic systems, mechanical systems, swelling systems, erosion controlled systems, and/or stimulated controlled release systems. A diffusion formulation system may include, for example, reservoir devices in which the oral therapeutic composition is encapsulated by a membrane barrier coat composed, for example, of one or more of hardened gelatin, methyl- or ethylcellulose, polyhydroxymethyacrylate, hydroxypropylcellulose, polyvinylacetate, and/or various waxes.

Alternatively, the diffusion formulation system may include matrix devices in which the oral therapeutic composition is uniformly dissolved or dispersed in an inert polymeric matrix composed, for example, of one or more plastic polymers (e.g., methyl acrylate-methyl methacrylate, polyvinyl chloride, or polyethylene); one or more hydrophilic polymers (e.g., methylcellulose, hydroxypropylmethylcellulose, sodium carboxymethylcellulose, or carbopol 934); one or more fatty compounds (e.g., carnauba wax or glyceryl tristearate), or both. The release rate of the therapeutic composition in a diffusion system is dependent upon the diffusion rate of the therapeutic composition in a diffusion system is dependent upon the diffusion rate of the therapeutic composition through the membrane barrier coat or polymeric matrix. A dissolution system can include, for example, similar formulation excipients, but in this instance the release rate of the therapeutic composition is dependent upon dissolution of the formulation, the therapeutic composition, or both. The dissolution rate can be controlled, for example, by one or more of adjusting the size of encapsulated drug particles, thickness of coating materials, or diffusivity of core materials.

### Example 8

### Composition Comprising Bortezomib and Imatinib

An intravenous therapeutic composition for treatment of malaria, other infections, cancer, autoimmune disease, allergic reactions, or other inflammatory reactions is generated containing a first agent that modulates the activity of one or more NF-kB molecules and a second agent that modulates the activity of one or more Src family kinases. The first agent is bortezomib ([(1R)-3-methyl-1-[[(2S)-1-oxo-3-phenyl-2-[(pyrazinylcarbonyl)amino]propyl]amino]butyl]boronic acid; C₁₉H₂₅BN₄O₄; molecular mass of 384.24 gm/mol), a modulator of NF-kB activity, and a proteasome inhibitor. The second agent is imatinib (4-[(4-methyl-1-piperazinyl)methyl]-N-[4-methyl-3-[[4-(3-pyridinyl)-2-pyrimidinyl]amino]-phenyl]benzamide methanesulfonate; C₂₉H₃₁N₇O.CH₄SO₃; molecular mass of 589.7 g/mol), a modulator of Src family kinase activity. A composition containing bortezomib and imatinib is formulated for intravenous administration.

The therapeutic composition contains a first agent and a second agent that constitute the active ingredients of the therapeutic composition. The active ingredients bortezomib and imatinib, for example, are combined in aqueous solution. In some instances, the aqueous solution containing bortezomib and imatinib is sterilized and directly apportioned into injection vials. The aqueous solution is then ready for immediate use. Alternatively, the aqueous solution containing bortezomib and imatinib is freeze-dried directly into injection vials. The freeze-dried powder is reconstituted prior to intravenous infusion. One or more injection vials containing quinine sulfate and bortezomib may be used over the course of treatment.

Each injection vial of the intravenous dosage form composition containing bortezomib and imatinib includes at least one dose for a 70 kilogram adult of about 2.2 mg bortezomib and about 800 mg of imatinib. Alternative dosage forms may include the same relative amounts of bortezomib and imatinib, but in smaller quanities. For example, the dosage form may contain bortezomib and imatinib in amounts of about 1.1 mg/400 mg, about 0.55 mg/200 mg, about 0.28 mg/100 mg, etc., respectively. Alternative dosage forms may be generated to include different relative amounts of bortezomib and imatinib. Alternative dosage forms may be determined empirically.

The intravenous dosage form composition containing bortezomib and imatinib may include additional inactive ingredients or excipients such as, for example, antimicrobial agents, buffers, antioxidants, tonicity agents, and or cryoprotectants and lyoprotectants. Antimicrobial agents in bacteriostatic or fungistatic concentrations may be added to preparations of multiple dose preparations to prevent possible microbial growth inadvertently introduced during withdrawal of a portion of the vial contents. Common examples of antimicrobial agents include phenylmercuric nitrate, thimerosal, benzethonium chloride, benzalkonium chloride, phenol, cresol and or chlorobutanol. Buffers are used to stabilize a solution against chemical or physical degradation. Common acid salts used as buffers include citrates, acetates and phosphates. Antioxidants are used to preserve products against oxidation. Common examples of antioxidants include sodium bisulfite, ascorbic acid, and salts thereof. Tonicity agents are used to ensure that injected material is isotonic with physiological fluids. Common examples of tonicity agents include electrolytes and monosaccharides or disaccharides. Cryoprotectants and lyoprotectants are additives that protect active ingredients from damage due to the freeze-drying process. Common cryoprotectant and lyoprotectant agents include sugars, amino acids, polymers, and polyols. As such, the single intravenous dosing form of bortezomib and imatinib may include one or more of these inactive ingredients, depending upon whether the dosing form is a solution or a freeze-dried powder.

For administration of the freeze-dried powder, the powder is reconstituted in an appropriate aqueous vehicle prior to initiating intravenous administration. An appropriate aqueous vehicle can be highly purified and sterile water or Water for Injection (WFI). The latter is prepared by distillation or by membrane technologies such as reverse osmosis or ultrafiltration. Alternatively, the freeze dried power is reconstituted with a physiologically appropriate vehicle such as sodium chloride or saline solution (0.9%), Ringer's solution, dextrose solution, lactated Ringer's solution, or dextrose and sodium chloride (0.9%) solution. The reconstituted solution of bortezomib and imatinib is administered as a bolus intravenous injection. Alternatively, bortezomib and imatinib are infused over the course of several hours using an infusion pump or an intravenous fluid bag.

In some instances, flexibility in the dosing of bortezomib and imatinib may be needed to effectively treat a subject with malaria, other infections, allergy, autoimmune disease, or other inflammatory reactions. For example, the appropriate dose of bortezomib and/or imatinib may be dependent upon one or more characteristic of the subject such as, for example, body weight (kilogram, kg), body surface area (meters squared, m²), gender, age, overall health status and severity of disease. The recommended intravenous dose of bortezomib is about 1.3 mg/m² or about 0.03 mg/kg. As such, only a portion of an intravenous dosage form containing about 2.2 mg of bortezomib and about 800 mg of imatinib, for example, may be administered by infusion over about a 24 hour period, depending upon the one or more characteristic of the subject. The intravenous dose composition containing bortezomib and imatinib may be administered using an infusion pump or an intravenous fluid bag filled with a physiological solution such as standard saline solution.

The composition containing bortezomib and imatinib may be administered by other parenteral dosing routes such as, for example, intramuscular or subcutaneous injection using, for example, the above-referenced dosages and formulations.

### Example 9

### Composition Comprising Disulfiram, Dasatinib, and Nilotinib

An intramuscular or subcutaneous therapeutic composition for treatment of malaria, viral infections, bacterial infections, allergy, autoimmune disease, or other inflammatory reactions is generated containing a first agent that modulates the activity of one or more NF-kB molecules, and two second agents that modulate the activity of one or more Src family kinases. The first agent is disulfiram (1-(diethylthiocarbamoyldisulfanyl)-N,N-diethyl-methanethioamide; C₁₀H₂₀N₂S₄; molecular mass of 296.53 gm/mol), a modulator of NF-kB activity. The two second agents are dasatinib (N-(2-chloro-6-methylphenyl)-2-[[6-[4-(2-hydroxyethyl)-1-piperazinyl]-2-methyl-4-pyrimidinyl]amino]-5-thiazolecarboxamide, monohydrate; C₂₂H₂₆ClN₇O₂S.H₂O; molecular mass of 488.01 g/mol) and nilotinib (4-methyl-N-[3-(4-methyl-1H-imidazol-1-yl)-5-(trifluoromethyl)phenyl]-3-[[4-(3-pyridinyl)-2-pyrimidinyl]amino]-benzamide, monohydrochloride, monohydrate; C₂₈H₂₂F₃N₇O. HCl.H₂O; molecular mass of 565.98 gm/mol), modulators of Src family kinase activity. A composition containing disulfiram, dasatinib, and nilotinib is formulated as a suspension for intramuscular or subcutaneous administration. Because the suspended disulfiram, dasatinib, and nilotinib may need to undergo dissolution prior to crossing biological membranes, a suspension formulation may provide sustained release of the agents.

The therapeutic composition contains a first and two second agents that constitute the active ingredients of the therapeutic composition. The active ingredients disulfiram, dasatinib, and nilotinib, for example, are combined in a parenteral dosage form such as, for example, an aqueous suspension. An aqueous suspension for dosing an adult would include about 250 mg/ml of disulfiram, about 400 mg/ml of nilotinib, and about 70 mg/ml of dasatinib. The suspension may be administered by either intramuscular or subcutaneous injection every about 12 hours, at a volume of about 1 ml, over the course of about 3 to about 10 days, for example.

The treatment course or regimen can include from about 1 day to about 28 days; from about 1 day to about 21 days; from about 1 day to about 14 days; from about 1 day to about 7 days; from about 3 days to about 28 days; from about 3 to about 21 days; from about 3 to about 14 days; from about 3 to about 7 days; from about 5 to about 28 days; from about 5 to about 21 days; from about 5 to about 14 days; from about 5 to about 7 days; or any length of time therebetween or greater.

Smaller doses of the aqueous suspension containing disulfiram, dasatinib, and nilotinib may be contemplated for use in more or less severe disease or in the pediatric population and may be accomplished by decreasing the injection volume. Alternatively, an aqueous suspension may be generated containing more or less of each compound. As such, the aqueous suspension that includes disulfiram, dasatinib, and nilotinib may contain an amount of disulfiram ranging from about 10 mg to about 500 mg, an amount of dasatinib ranging from about 10 mg to about 140 mg, and an amount of nilotinib ranging from about 10 mg to about 800 mg. An aqueous suspension containing larger doses of disulfiram, dasatinib, and nilotinib may also be generated. Alternatively, the amount of disulfiram, dasatinib, and nilotinib in the composition may be determined empirically.

The parenteral dosage form composition containing disulfiram, dasatinib, and nilotinib may include additional inactive ingredients or excipients such as anionic and nonionic cellulose derivatives, anionic and nonionic natural polymers such as polysaccharides, anionic and nonionic synthetic polymers such as cross-linked polyacrylates, and clays. These excipients may function as flocculating/stabilizing and viscosity enhancing agents. Common examples include carboxymethylcellulose (CMC), microcrystalline cellulose, hydroxypropyl-methylcellulose (HPMC), acacia, carageenan, polyvinylpyrrolidone (PVP), and magnesium aluminum silicate. In some instances, a wetting agent such as an alcohol, glycerin or non-ionic surfactants such as Cremophor EL and polysorbate 80 (Tween 80) may be used to first wet the dry powder, particulate active ingredients prior to suspension in other excipients.

A suspension containing disulfiram, dasatinib, and nilotinib may be generated by first combining dry powder of each active ingredient into a mortar. The dry powders may have been micronized to reduce the particle size and to facilitate better in vivo dissolution. The dry powders are ground together in the mortar using a pestle and wetted with a small volume of a wetting agent such as, for example, polysorbate 80. To this slurry is slowly added about a 1% to 4% w/v solution of hydroxypropyl-methylcellulose and other appropriate excipients in aqueous buffer to generate a suspension containing the active ingredients. The suspension is used for intramuscular or subcutaneous injection. Alternatively, the suspension may be used for oral administration.

The composition containing disulfiram, dasatinib, and nilotinib may be administered by other parenteral dosing routes such as, for example, intramuscular or subcutaneous injection using, for example, the above-referenced dosages and formulations.

### Example 10

### Composition Comprising Quinine Sulfate, Dasatinib, and Disulfiram

An oral therapeutic composition for treatment of malaria, viral infections, bacterial infections, allergy, autoimmune disease, cancer, or other inflammatory reactions is generated containing a first agent that modulates the activity of one or more Toll-like receptors, a second agent that modulates the activity of one or more Src family kinases, and third agent that modulates the activity of one or more NF-kB molecules. The first agent is quinine sulfate (cinchonan-9-ol, 6′-methoxy-, (8.alpha.,9R)-, sulfate (2:1) (salt); C₂₀H₂₄N₂O₂)₂.H₂SO₄.2H₂O); molecular weight 782.96), a modulator of Toll-like receptor activity. The second agent is dasatinib (N-(2-chloro-6-methylphenyl)-2-[[6-[4-(2-hydroxyethyl)-1-piperazinyl]-2-methyl-4-pyrimidinyl]amino]-5-thiazolecarboxamide, monohydrate; C₂₂H₂₆ClN₇O₂S.H₂O; molecular mass of 488.01 g/mol), a modulator of Src family kinase activity. The third agent is disulfiram (1-(diethylthiocarbamoyl-disulfanyl)-N,N-diethyl-methanethioamide; C₁₀H₂₀N₂S₄; molecular mass of 296.53 gm/mol), a modulator of NF-kB activity. A composition containing quinine sulfate, dasatinib and disulfiram is formulated for oral administration. The therapeutic composition is formulated to enable sufficient dissolution and absorption of the first, the second, and the third agent to achieve adequate oral bioavailability and systemic dosing.

The therapeutic composition contains a first, a second and a third agent that constitute the active ingredients of the therapeutic composition. The active ingredients quinine sulfate, dasatinib and disulfiram, for example, are combined in a single oral solid dosage form for oral administration. The oral solid dosage form constitutes one or more tablets. Alternatively the oral solid dosage form constitutes one or more of a hard or soft gelatin capsule. The oral solid dosage form is taken by a subject or administered to a subject on a periodic basis. For example, tablets containing quinine sulfate, dasatinib, and disulfiram may be administered at least once daily over the course of about 8 to about 10 days, for example, to treat malaria and other inflammatory reactions.

The treatment course or regimen can include from about 1 day to about 28 days; from about 1 day to about 21 days; from about 1 day to about 14 days; from about 1 day to about 7 days; from about 3 days to about 28 days; from about 3 to about 21 days; from about 3 to about 14 days; from about 3 to about 7 days; from about 5 to about 28 days; from about 5 to about 21 days; from about 5 to about 14 days; from about 5 to about 7 days; or any length of time therebetween or greater.

Each dose of the composition containing quinine sulfate, dasatinib, and disulfiram formulated for an adult would include about 648 mg of quinine sulfate, about 70 mg of dasatinib, and about 250 mg of disulfiram and be administered every about 12 hours, for example. Alternatively, it may be beneficial to administer the combination of quinine sulfate, dasatinib, and disulfiram as two or more tablets, two or more times per day over the course of about 8 to about 10 days, for example. In this instance, each tablet contains about 324 mg of quinine sulfate, about 35 mg of dasatinib, and about 125 mg of disulfiram. Tablets containing smaller amounts of quinine sulfate, dasatinib, and disulfiram may be useful for treating less severe disease or smaller subjects such as, for example, pediatric subjects. For example, quinine sulfate is administered as a single agent at about 10 mg/kg in the pediatric population. Similarly, dasatinib has been administered as a single agent in the pediatric population at doses ranging from about 60 to about 160 mg/m² (or approximately 2-5 mg/kg) (See, e.g., Porkka, et al., Blood 112:1005-1012, 2008, which is herein incorporated by reference). As such, the combination oral dosage form intended for administration at least once daily may contain an amount of quinine sulfate ranging from about 10 mg to about 1296 mg, an amount of dasatinib ranging from about 10 mg to about 140 mg, and an amount of disulfiram ranging from about 10 mg to about 500 mg. Tablets containing larger doses of quinine sulfate, dasatinib, and/or disulfiram may also be generated. Alternative compositions containing quinine sulfate, dasatinib, and disulfiram may be determined empirically.

The single oral dosage form containing quinine sulfate, dasatinib, and disulfiram may also include a number of inactive ingredients or excipients. For example, the tablets may include excipients that are one or more of fillers, binders, lubricants, disintegrants, or combinations thereof. In some instances, a single excipient may have multiple functionalities in the formulation. Fillers are used primarily to create a pill volume that is sufficiently large enough for human fingers to readily handle. Common examples of fillers include lactose, microcrystalline cellulose, corn starch, and sugars such as mannitol, sorbitol, fructose, and dextrose. Binders are used to impart cohesiveness to the tablet formulation that ensures the tablet remains intact after compression. Common examples of binders include starch, gelatin, sugars, and natural and synthetic gums such as acacia and methylcellulose. Lubricants also aide in tablet compression and further prevent the tablets from adhering to the walls of the tablet forming molds. Common examples of lubricants include magnesium stearate, stearic acid, talc, sodium stearyl fumarate and hydrogenated vegetable oil. Polyethylene glycol may also be used to allow the tablet to drop more readily out of the mold. Disintegrants facilitate the dissolution of the tablet in the gastrointestinal tract. Common examples of disintegrants include crospovidone, croscarmellose sodium, and gellan gum. As such, quinine sulfate, dasatinib, and disulfiram are formulated in tablet form and may include one or more of the following inactive ingredients: lactose monohydrate, microcrystalline cellulose, croscarmellose sodium, povidone, hydroxypropyl cellulose, magnesium aluminum silicate, magnesium stearate, corn starch and talc.

The oral dosage form containing quinine sulfate, dasatinib, and disulfiram may also include a coating that prevents the tablet from dissolving prematurely and may mask an objectionable taste and or smell of the active ingredients. Quinine in particular has a distinctive bitter taste. As such, tablets containing quinine sulfate, dasatinib, and disulfiram are further coated with hypromellose, titanium dioxide, and polyethylene glycol with optional color additives of red and or yellow iron oxides.

In general, the inactive ingredients or excipients included in the single oral dosage form of quinine sulfate, dasatinib, and disulfiram and other drug dosing combinations described herein are approved for use in human subjects by the Food and Drug Administration (FDA) and are listed in either the United States Pharmacopeia (USP) or National Formulary (NF) for products sold in the United States, or the European Pharmacopeia (EP) for products sold in Europe.

The oral therapeutic composition containing quinine sulfate, dasatinib, and disulfiram can be formulated for delayed release. Delayed release permits repetitive, intermittent dosing of the composition from one or more immediate-release units incorporated into a dosage form, for example, repeat-action tablets or capsules. One example includes multilayer or multi-component tablets, caplets or capsules in which each layer or component dissolves or disintegrates to release one or more component of the therapeutic composition. Alternatively, delayed release can include utilizing an enteric delayed release system in which the therapeutic composition is coated with one or more pH sensitive polymer that remains intact in the acidic environment of the stomach and then solubilizes or disintegrates in the more alkaline environment of the small intestine. Polymers used for this purpose include, for example, cellulose acetate phthalate, polyvinylacetate phthalate, hydroxypropylmethylcellulose phthalate, methacrylic acid-methacrylic acid ester copolymers, cellulose acetate trimellitate, carboxymethyl ethylcellulose, or hydroxypropyl methylcellulose acetate succinate.

Alternatively, the oral therapeutic composition containing quinine sulfate, dasatinib, and disulfiram can be formulated for extended release to maintain therapeutic blood or tissue levels of the therapeutic composition for a prolonged period of time. Extended release formulations include, for example, diffusion systems, dissolution systems, osmotic systems, mechanical systems, swelling systems, erosion controlled systems, and/or stimulated controlled release systems. A diffusion formulation system may include, for example, reservoir devices in which the oral therapeutic composition is encapsulated by a membrane barrier coat composed, for example, of one or more of hardened gelatin, methyl- or ethylcellulose, polyhydroxymethyacrylate, hydroxypropylcellulose, polyvinylacetate, and/or various waxes.

Alternatively, the diffusion formulation system may include matrix devices in which the oral therapeutic composition is uniformly dissolved or dispersed in an inert polymeric matrix composed, for example, of one or more plastic polymers (e.g., methyl acrylate-methyl methacrylate, polyvinyl chloride, or polyethylene); one or more hydrophilic polymers (e.g., methylcellulose, hydroxypropylmethylcellulose, sodium carboxymethylcellulose, or carbopol 934); one or more fatty compounds (e.g., carnauba wax or glyceryl tristearate), or both. The release rate of the therapeutic composition in a diffusion system is dependent upon the diffusion rate of the therapeutic composition in a diffusion system is dependent upon the diffusion rate of the therapeutic composition through the membrane barrier coat or polymeric matrix. A dissolution system can include, for example, similar formulation excipients, but in this instance the release rate of the therapeutic composition is dependent upon dissolution of the formulation, the therapeutic composition, or both. The dissolution rate can be controlled, for example, by one or more of adjusting the size of encapsulsted drug particles, thickness of coating materials, or diffusivity of core materials.

### Example 11

### Composition Comprising Chloroquine Phosphate, Imatinib, and Bortezomib

An intravenous therapeutic composition for treatment of malaria, viral infections, bacterial infections, sepsis, systemic inflammatory response syndrome, septic shock, multiple organ dysfunction syndrome, multiple organ dysfunction syndrome, autoimmune disease, allergy, or other inflammatory reactions is generated containing a first agent that modulates the activity of one or more Toll-like receptors, a second agent that modulates the activity of one or more Src family kinases, and a third agent that modulates the activity of one or more NF-kB molecules. The first agent is chloroquine phosphate (7-chloro-4-[[4-(diethylamino)-1-methylbutyl]amino]quinoline phosphate (1:2); C₁₈H₂₆ClN₃.2H₃PO₄; molecular weight 515.86), a modulator of Toll-like receptor activity. The second agent is imatinib (4-[(4-methyl-1-piperazinyl)methyl]-N-[4-methyl-3-[[4-(3-pyridinyl)-2-pyrimidinyl]amino]-phenyl]benzamide methanesulfonate; C₂₉H₃₁N₇O.CH₄SO₃; molecular mass of 589.7 g/mol), a modulator of Src family kinase activity. The third agent is bortezomib ([(1R)-3-methyl-1-[[(2S)-1-oxo-3-phenyl-2-[(pyrazinylcarbonyl)amino]propyl]amino]butyl]boronic acid; C₁₉H₂₅BN₄O₄; molecular mass of 384.24 gm/mol), a modulator of NF-kB activity, and a proteasome inhibitor. A composition containing chloroquine phosphate, imatinib, and bortezomib is formulated for intravenous administration.

The therapeutic composition contains a first agent, a second agent, and a third agent that constitute the active ingredients of the therapeutic composition. The active ingredients chloroquine phosphate, imatinib, and bortezomib, for example, are combined in an aqueous solution. In some instances, the aqueous solution containing chloroquine phosphate, imatinib, and bortezomib is sterilized and directly apportioned into injection vials. The aqueous solution is then ready for immediate use. Alternatively, the aqueous solution containing chloroquine phosphate, imatinib, and bortezomib is freeze-dried directly into injection vials. The freeze-dried powder is resolubilized prior to intravenous injection or infusion. One or more injection vial containing chloroquine phosphate, imatinib, and bortezomib may be used over the course of infusion treatment.

Each injection vial of the intravenous dosage form composition containing chloroquine phosphate, imatinib, and bortezomib includes at least one dose for a 70 kilogram adult of about 1400 mg chloroquine phosphate, about 800 mg of imatinib, and about 2.2 mg of bortezomib, for example. Alternative dosage forms may include the same relative amounts of chloroquine phosphate, imatinib, and bortezomib, but in small quantities. For example, the dosage form may contain chloroquine phosphate, imatinib, and bortezomib in amounts of about 700 mg/400 mg/1.1 mg, about 350 mg/200 mg/0.55 mg, about 175 mg/100 mg/0.28 mg, etc., respectively. Alternative dosage forms may be contemplated to include different relative amounts of each compound. Alternative dosage forms may be determined empirically.

The intravenous dosage form composition containing chloroquine phosphate, imatinib, and bortezomib may include additional inactive ingredients or excipients such as, for example, antimicrobial agents, buffers, antioxidants, tonicity agents, and or cryoprotectants and lyoprotectants. Antimicrobial agents in bacteriostatic or fungistatic concentrations may be added to preparations of multiple dose preparations to prevent possible microbial growth inadvertently introduced during withdrawal of a portion of the vial contents. Common examples of antimicrobial agents include phenylmercuric nitrate, thimerosal, benzethonium chloride, benzalkonium chloride, phenol, cresol and or chlorobutanol. Buffers are used to stabilize a solution against chemical or physical degradation. Common acid salts used as buffers include citrates, acetates and phosphates. Antioxidants are used to preserve products against oxidation. Common examples of antioxidants include sodium bisulfite, ascorbic acid, and salts thereof. Tonicity agents are used to ensure that injected material is isotonic with physiological fluids. Common examples of tonicity agents include electrolytes and monosaccharides or disaccharides. Cryoprotectants and lyoprotectants are additives that protect active ingredients from damage due to the freeze-drying process. Common cryoprotectant and lyoprotectant agents include sugars, amino acids, polymers, and polyols. As such, the single intravenous dosing form of chloroquine phosphate, imatinib, and bortezomib may include one or more of these inactive ingredients, depending upon whether the dosing form is a solution or a freeze-dried powder. For example, a chloroquine phosphate, imatinib, and bortezomib intravenous dosage form may include mannitol, a sugar alcohol polyol.

For administration of the freeze-dried powder, the powder is reconstituted in an appropriate aqueous vehicle prior to initiating intravenous administration. An appropriate aqueous vehicle can be highly purified and sterile water or Water for Injection (WFI). The latter is prepared by distillation or by membrane technologies such as reverse osmosis or ultrafiltration. Alternatively, the freeze dried power is reconstituted with a physiologically appropriate vehicle such as sodium chloride or saline solution (0.9%), Ringer's solution, dextrose solution, lactated Ringer's solution, or dextrose and sodium chloride (0.9%) solution. The reconstituted solution of chloroquine phosphate, imatinib, and bortezomib is administered as a bolus intravenous injection. Alternatively, chloroquine phosphate, imatinib, and bortezomib are infused over the course of several hours using an infusion pump or an intravenous fluid bag.

In some instances, flexibility in the dosing of chloroquine phosphate, imatinib, and bortezomib may be needed to effectively treat a subject with malaria, sepsis, systemic inflammatory response syndrome, septic shock, multiple organ dysfunction syndrome, allergy, autoimmune disease, other infections, or other inflammatory reactions. For example, the appropriate dose of chloroquine phosphate, imatinib, and/or bortezomib may be dependent upon one or more characteristic of the subject such as, for example, body weight (kilogram, kg), body surface area (meters squared, m²), gender, age, overall health status and severity of disease. For example, the recommended intravenous dose of chloroquine phosphate ranges from about 10 to about 20 mg/kg in about a 24 hour period. The recommended intravenous dose of bortezomib is about 1.3 mg/m² or about 0.03 mg/kg. As such, only a portion of an intravenous dosage form containing about 1400 mg of chloroquine phosphate, about 800 mg of imatinib, and about 2.2 mg bortezomib, for example, may be administered by infusion over about a 24 hour period, depending upon the one or more characteristic of the subject. The intravenous dose composition containing chloroquine phosphate, imatinib, and bortezomib may be administered using an infusion pump or an intravenous fluid bag filled with a physiological solution such as standard saline solution.

The composition containing chloroquine phosphate, imatinib, and bortezomib may be administered by other parenteral dosing routes such as, for example, intramuscular or subcutaneous injection using, for example, the above-referenced dosages and formulations.

### Example 12

### Composition Comprising Chloroquine Phosphate, Nilotinib, and Disulfiram

An intramuscular or subcutaneous therapeutic composition for treatment of malaria, viral infections, bacterial infections, sepsis, systemic inflammatory response syndrome, septic shock, multiple organ dysfunction syndrome, allergy, autoimmune disease, cancer, or other inflammatory reactions is generated containing a first agent that modulates the activity of one or more Toll-like receptors, a second agent that modulates the activity of one or more Src family kinases, and a third agent that modulates the activity of one or more NF-kB molecules. The first agent is chloroquine phosphate (7-chloro-4-[[4-(diethylamino)-1-methylbutyl]amino]quinoline phosphate (1:2); C₁₈H₂₆ClN₃.2H₃PO₄; molecular weight 515.86), a modulator of Toll-like receptor activity. The second agent is nilotinib (4-methyl-N-[3-(4-methyl-1H-imidazol-1-yl)-5-(trifluoromethyl)phenyl]-3-[[4-(3-pyridinyl)-2-pyrimidinyl]amino]-benzamide, monohydrochloride, monohydrate; C₂₈H₂₂F₃N₇O.HCl.H₂O; molecular mass of 565.98 gm/mol), a modulator of Src family kinase activity. The third agent is disulfiram (1-(diethylthiocarbamoyldisulfanyl)-N,N-diethyl-methanethioamide; C₁₀H₂₀N₂S₄; molecular mass of 296.53 gm/mol), a modulator of NF-kB activity. A composition containing chloroquine phosphate, nilotinib, and disulfiram is formulated as a suspension for intramuscular or subcutaneous administration. Because the suspended chloroquine phosphate, nilotinib, and disulfiram may need to undergo dissolution prior to crossing biological membranes, a suspension formulation may provide sustained release of the agents.

The therapeutic composition contains a first agent, a second agent, and a third agent that constitute the active ingredients of the therapeutic composition. The active ingredients chloroquine phosphate, nilotinib, and disulfiram, for example, are combined a parenteral dosage form such as, for example, an aqueous suspension. An aqueous suspension for dosing an adult would include about 1400 mg/ml chloroquine phosphate, about 400 mg/ml nilotinib, and about 250 mg/ml disulfiram. The suspension may be administered by either intramuscular or subcutaneous injection about every 12 hours, at a volume of about 1 ml, over the course of about 3 to about 10 days, for example.

The treatment course or regimen can include from about 1 day to about 28 days; from about 1 day to about 21 days; from about 1 day to about 14 days; from about 1 day to about 7 days; from about 3 days to about 28 days; from about 3 to about 21 days; from about 3 to about 14 days; from about 3 to about 7 days; from about 5 to about 28 days; from about 5 to about 21 days; from about 5 to about 14 days; from about 5 to about 7 days; or any length of time therebetween (e.g. a fraction of a day) or greater.

Smaller doses of the aqueous suspension containing chloroquine phosphate, nilotinib, and disulfiram may be contemplated for use in more or less severe disease or in the pediatric population and may be accomplished by decreasing the injection volume. Alternatively, an aqueous suspension may be generated containing more or less of each compound. As such, the aqueous suspension that includes chloroquine phosphate, nilotinib, and disulfiram may contain an amount of chloroquine phosphate ranging from about 10 mg to about 1400 mg, and an amount of nilotinib ranging from about 10 mg to about 800 mg, and an amount of disulfiram ranging from about 10 mg to about 500 mg. An aqueous suspension containing larger doses of chloroquine phosphate, nilotinib, and disulfiram may also be generated. Alternatively, the amount of chloroquine phosphate, nilotinib, and disulfiram in the composition may be determined empirically.

The parenteral dosage form composition containing chloroquine phosphate, nilotinib, and disulfiram may include additional inactive ingredients or excipients such as anionic and nonionic cellulose derivatives, anionic and nonionic natural polymers such as polysaccharides, anionic and nonionic synthetic polymers such as cross-linked polyacrylates, and clays. These excipients may function as flocculating/stabilizing and viscosity enhancing agents. Common examples include carboxymethylcellulose (CMC), microcrystalline cellulose, hydroxypropyl-methylcellulose (HPMC), acacia, carageenan, polyvinylpyrrolidone (PVP), and magnesium aluminum silicate. In some instances, a wetting agent such as an alcohol, glycerin or non-ionic surfactants such as Cremophor EL and polysorbate 80 (Tween 80) may be used to first wet the dry powder, particulate active ingredients prior to suspension in other excipients.

A suspension containing chloroquine phosphate, nilotinib, and disulfiram is generated by first combining dry powder of each active ingredient into a mortar. The dry powders may have been micronized to reduce the particle size and to facilitate better in vivo dissolution. The dry powders are ground together in the mortar using a pestle and wetted with a small volume of a wetting agent such as, for example, polysorbate 80. To this slurry is slowly added about a 1% to 4% w/v solution of hydroxypropylmethylcellulose and other appropriate excipients in aqueous buffer to generate a suspension containing the active ingredients. The suspension is used for intramuscular or subcutaneous injection. The composition containing chloroquine phosphate, nilotinib, and disulfiram may be administered by other parenteral dosing routes such as, for example, intramuscular or subcutaneous injection using, for example, the above-referenced dosages and formulations. Alternatively, the suspension is used for oral administration.

The oral therapeutic composition containing chloroquine phosphate, nilotinib, and disulfiram can be formulated for delayed release. Delayed release permits repetitive, intermittent dosing of the composition from one or more immediate-release units incorporated into a dosage form, for example, repeat-action tablets or capsules. One example includes multilayer or multi-component tablets, caplets or capsules in which each layer or component dissolves or disintegrates to release one or more component of the therapeutic composition. Alternatively, delayed release can include utilizing an enteric delayed release system in which the therapeutic composition is coated with one or more pH sensitive polymer that remains intact in the acidic environment of the stomach and then solubilizes or disintegrates in the more alkaline environment of the small intestine. Polymers used for this purpose include, for example, cellulose acetate phthalate, polyvinylacetate phthalate, hydroxypropylmethylcellulose phthalate, methacrylic acid-methacrylic acid ester copolymers, cellulose acetate trimellitate, carboxymethyl ethylcellulose, or hydroxypropyl methylcellulose acetate succinate.

Alternatively, the oral therapeutic composition containing chloroquine phosphate, nilotinib, and disulfiram can be formulated for extended release to maintain therapeutic blood or tissue levels of the therapeutic composition for a prolonged period of time. Extended release formulations include, for example, diffusion systems, dissolution systems, osmotic systems, mechanical systems, swelling systems, erosion controlled systems, and/or stimulated controlled release systems. A diffusion formulation system may include, for example, reservoir devices in which the oral therapeutic composition is encapsulated by a membrane barrier coat composed, for example, of one or more of hardened gelatin, methyl- or ethylcellulose, polyhydroxymethyacrylate, hydroxypropylcellulose, polyvinylacetate, and/or various waxes.

Alternatively, the diffusion formulation system may include matrix devices in which the oral therapeutic composition is uniformly dissolved or dispersed in an inert polymeric matrix composed, for example, of one or more plastic polymers (e.g., methyl acrylate-methyl methacrylate, polyvinyl chloride, or polyethylene); one or more hydrophilic polymers (e.g., methylcellulose, hydroxypropylmethylcellulose, sodium carboxymethylcellulose, or carbopol 934); one or more fatty compounds (e.g., carnauba wax or glyceryl tristearate), or both. The release rate of the therapeutic composition in a diffusion system is dependent upon the diffusion rate of the therapeutic composition in a diffusion system is dependent upon the diffusion rate of the therapeutic composition through the membrane barrier coat or polymeric matrix. A dissolution system can include, for example, similar formulation excipients, but in this instance the release rate of the therapeutic composition is dependent upon dissolution of the formulation, the therapeutic composition, or both. The dissolution rate can be controlled, for example, by one or more of adjusting the size of encapsulsted drug particles, thickness of coating materials, or diffusivity of core materials.

### Example 13

### Composition Comprising Quinine Sulfate, Dasatinib, Nilotinib, and Disulfiram

An oral therapeutic composition for treatment of malaria, viral infections, bacterial infections, sepsis, systemic inflammatory response syndrome, septic shock, multiple organ dysfunction syndrome, allergy, autoimmune disease, other parasitic infections, cancer, or other inflammatory reactions is generated containing a first agent that modulates the activity of one or more Toll-like receptors, two second agents that modulate the activity of one or more Src family kinases, and a third agent that modulates the activity of one or more NF-kB molecules. The first agent is quinine sulfate (cinchonan-9-ol, 6′-methoxy-, (8.alpha.,9R)-, sulfate (2:1) (salt); C₂₀H₂₄N₂O₂)₂.H₂SO₄.2H₂O); molecular weight 782.96), a modulator of Toll-like receptor activity. The two second agents are dasatinib (N-(2-chloro-6-methylphenyl)-2-[[6-[4-(2-hydroxyethyl)-1-piperazinyl]-2-methyl-4-pyrimidinyl]amino]-5-thiazolecarboxamide, monohydrate; C₂₂H₂₆ClN₇O₂S.H₂O; molecular mass of 488.01 g/mol) and nilotinib (4-methyl-N-[3-(4-methyl-1H-imidazol-1-yl)-5-(trifluoromethyl)phenyl]-3-[[4-(3-pyridinyl)-2-pyrimidinyl]amino]-benzamide, monohydrochloride, monohydrate; C₂₈H₂₂F₃N₇O.HCl.H₂O; molecular mass of 565.98 gm/mol), modulators of Src family kinase activity. The third agent is disulfiram (1-(diethylthiocarbamoyldisulfanyl)-N,N-diethyl-methanethioamide; C₁₀H₂₀N₂S₄; molecular mass of 296.53 gm/mol), a modulator of NF-kB activity. A composition containing quinine sulfate, dasatinib, nilotinib, and disulfiram is formulated for oral administration. The therapeutic composition is formulated to enable sufficient dissolution and absorption of the first and second agents to achieve adequate oral bioavailability and systemic dosing.

The therapeutic composition contains a first agent, two second agents and a third agent that constitute the active ingredients of the therapeutic composition. The active ingredients quinine sulfate, dasatinib, nilotinib, and disulfiram, for example, are combined in a single oral solid dosage form for oral administration. The oral solid dosage form constitutes one or more tablets. Alternatively the oral solid dosage form constitutes one or more of a hard or soft gelatin capsule. The oral solid dosage form is taken by a subject or administered to a subject on a periodic basis. For example, tablets containing quinine sulfate, dasatinib, nilotinib, and disulfiram may be administered at least once daily, over the course of about 8 to about 10 days, for example, to treat malaria, other infections, sepsis, systemic inflammatory response syndrome, septic shock, multiple organ dysfunction, allergy, autoimmune disease, cancer, or other inflammatory reactions.

The treatment course or regimen can include from about 1 day to about 28 days; from about 1 day to about 21 days; from about 1 day to about 14 days; from about 1 day to about 7 days; from about 3 days to about 28 days; from about 3 to about 21 days; from about 3 to about 14 days; from about 3 to about 7 days; from about 5 to about 28 days; from about 5 to about 21 days; from about 5 to about 14 days; from about 5 to about 7 days; or any length of time therebetween or greater.

Each dose of the composition containing quinine sulfate, dasatinib, nilotinib, and disulfiram formulated for an adult would include about 648 mg of quinine sulfate, about 70 mg of dasatinib, about 400 mg of nilotinib, and about 250 mg of disulfiram and be administered about every 12 hours, for example. In some instances, it may be beneficial to administer the composition including quinine sulfate, dasatinib, nilotinib, and disulfiram as two or more tablets, two or more times per day over the course of about 8 to about 10 days, for example.

The treatment course or regimen can include from about 1 day to about 28 days; from about 1 day to about 21 days; from about 1 day to about 14 days; from about 1 day to about 7 days; from about 3 days to about 28 days; from about 3 to about 21 days; from about 3 to about 14 days; from about 3 to about 7 days; from about 5 to about 28 days; from about 5 to about 21 days; from about 5 to about 14 days; from about 5 to about 7 days; or any length of time therebetween or greater.

In this instance, each tablet contains about 324 mg of quinine sulfate, about 35 mg of dasatinib, about 200 mg of nilotinib, and about 125 mg of disulfiram. Dosage forms containing more or less of each compound may also be contemplated for use in more or less severe disease or in the pediatric population, for example. As such, the combination oral dosage form intended for administration at least once daily may contain an amount of quinine sulfate ranging from about 10 mg to about 1296, an amount of dasatinib ranging from about 10 mg to about 140 mg, an amount of nilotinib ranging from about 10 mg to about 800 mg, and an amount of disulfiram ranging from about 10 mg to about 500 mg. Tablets containing larger doses of quinine sulfate, dasatinib, nilotinib, and disulfiram may also be generated. Alternatively, the amount of quinine sulfate, dasatinib, nilotinib, and disulfiram in the composition may be determined empirically.

The oral dosage form containing quinine sulfate, dasatinib, nilotinib, and disulfiram may also include a number of inactive ingredients or excipients, examples of which have been described herein. As such, quinine sulfate, dasatinib, nilotinib, and disulfiram are formulated in tablet form and may include one or more of the following inactive ingredients: lactose monohydrate, microcrystalline cellulose, croscarmellose sodium, hydroxypropyl cellulose, colloidal silicon dioxide, crospovidone, povidone, magnesium aluminum silicate, magnesium stearate, polyoxamer 188, corn starch, and talc.

The oral therapeutic composition containing quinine sulfate, dasatinib, nilotinib, and disulfiram can be formulated for delayed release. Delayed release permits repetitive, intermittent dosing of the composition from one or more immediate-release units incorporated into a dosage form, for example, repeat-action tablets or capsules. One example includes multilayer or multi-component tablets, caplets or capsules in which each layer or component dissolves or disintegrates to release one or more component of the therapeutic composition. Alternatively, delayed release can include utilizing an enteric delayed release system in which the therapeutic composition is coated with one or more pH sensitive polymer that remains intact in the acidic environment of the stomach and then solubilizes or disintegrates in the more alkaline environment of the small intestine. Polymers used for this purpose include, for example, cellulose acetate phthalate, polyvinylacetate phthalate, hydroxypropylmethylcellulose phthalate, methacrylic acid-methacrylic acid ester copolymers, cellulose acetate trimellitate, carboxymethyl ethylcellulose, or hydroxypropyl methylcellulose acetate succinate.

Alternatively, the oral therapeutic composition containing quinine sulfate, dasatinib, nilotinib, and disulfiram can be formulated for extended release to maintain therapeutic blood or tissue levels of the therapeutic composition for a prolonged period of time. Extended release formulations include, for example, diffusion systems, dissolution systems, osmotic systems, mechanical systems, swelling systems, erosion controlled systems, and/or stimulated controlled release systems. A diffusion formulation system may include, for example, reservoir devices in which the oral therapeutic composition is encapsulated by a membrane barrier coat composed, for example, of one or more of hardened gelatin, methyl- or ethylcellulose, polyhydroxymethyacrylate, hydroxypropylcellulose, polyvinylacetate, and/or various waxes.

Alternatively, the diffusion formulation system may include matrix devices in which the oral therapeutic composition is uniformly dissolved or dispersed in an inert polymeric matrix composed, for example, of one or more plastic polymers (e.g., methyl acrylate-methyl methacrylate, polyvinyl chloride, or polyethylene); one or more hydrophilic polymers (e.g., methylcellulose, hydroxypropylmethylcellulose, sodium carboxymethylcellulose, or carbopol 934); one or more fatty compounds (e.g., carnauba wax or glyceryl tristearate), or both. The release rate of the therapeutic composition in a diffusion system is dependent upon the diffusion rate of the therapeutic composition in a diffusion system is dependent upon the diffusion rate of the therapeutic composition through the membrane barrier coat or polymeric matrix. A dissolution system can include, for example, similar formulation excipients, but in this instance the release rate of the therapeutic composition is dependent upon dissolution of the formulation, the therapeutic composition, or both. The dissolution rate can be controlled, for example, by one or more of adjusting the size of encapsulated drug particles, thickness of coating materials, or diffusivity of core materials.

While particular aspects of the present subject matter described herein have been shown and described, it will be apparent that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

All publications and patent applications cited in this specification are herein incorporated by reference to the extent not inconsistent with the description herein and for all purposes as if each individual publication or patent application were specifically and individually indicated to be incorporated by reference for all purposes.

What is claimed is:

1. A system comprising:
at least one drug delivery device configured to retain and dispense at least one therapeutic composition to at least one subject; and
one or more instructions that when executed on a computing device cause the computing device to
regulate dispensing of the at least one drug delivery device,
wherein the delivery device includes at least one therapeutic composition including at least one first agent configured to modulate the activity of one or more NF-kB molecules; and
at least one second agent configured to modulate the activity of one or more Src family kinases.

2. The system of claim 1, wherein the at least one therapeutic composition further includes at least one pharmaceutically-acceptable carrier or excipient.

3. The system of claim 1, wherein the computing device includes one or more of a personal digital assistant (PDA), a laptop computer, a tablet personal computer, a networked computer, a computing system including a cluster of processors, a computing system including a cluster of servers, a mobile telephone, a workstation computer, or a desktop computer.

4. The system of claim 1, further comprising one or more instructions for determining at least one treatment regimen including modulating the activity of one or more NF-kB molecules, and one or more Src family kinases, based on at least one genetic or proteomic profile of the subject.

5. The system of claim 4, wherein the treatment regimen is configured to maintain a predetermined level of activity of one or more NF-kB molecules, and one or more Src family kinases in the subject.

6. The system of claim 4, further comprising one or more instructions for inputting information associated with physiological activity levels of one or more NF-kB molecules, and one or more Src family kinases in the subject.

7. The system of claim 1, wherein the at least one first agent includes one or more of disulfiram, ditiocarb, sulindac, sulfasalazine, or bortezomib.

8. The system of claim 1, wherein the at least one second agent includes one or more of dasatinib, nilotinib, BMSD-268770, UR-12947, aztreonam, MZ-338, riluzole, meloxicam, pramipexole, CBS-113-A, AZD0530, INNO-406, MK-0457, cediranib, sunitinib, bosutinib, axitinib, erlotinib, gefitinib, lapatinib, lestaurtinib, semaxanib, or imatinib.

9. The system of claim 1, wherein the at least one therapeutic composition further includes at least one third agent configured to modulate the activity of one or more Toll-like receptors.

10. The system of claim 9, wherein the at least one third agent includes at least one of chloroquine, M62812, or quinine.

11. The system of claim 1, wherein the at least one therapeutic composition further includes at least one fourth agent configured to modulate the activity of at least one protease or proteasome.

12. The system of claim 11, wherein the at least one fourth agent inhibits the activity of at least one protease.

13. The system of claim 12, wherein the at least one fourth agent includes one or more of saquinavir, ritonavir, indinavir, nelfinavir, amprenavir, lopinavir, atazanavir, fosamprenavir, tipranavir, or darunavir.

14. The system of claim 11, wherein the at least one fourth agent inhibits the activity of at least one proteasome.

15. The system of claim 14, wherein the at least one fourth agent includes dichloroisocoumarin or bortezomib.

16. The system of claim 11, wherein the at least one fourth agent includes one or more of an organic or inorganic small molecule, nucleic acid, amino acid, peptide, polypeptide, protein, glycopeptide, glycoprotein, glycolipid, lipopolysaccharide, peptidoglycan, proteoglycan, lipid, metalloprotein, liposome, or carbohydrate.

17. The system of claim 11, wherein the amount of one or more of the at least one first agent, the at least one second agent, the at least one third agent, or the at least one fourth agent are selected based on one or more attributes of the subject.

18. The system of claim 11, wherein the one or more attributes of the subject include phenotypic or genotypic attributes.

19. The system of claim 18, wherein the one or more attributes of the subject include one or more of a physiological condition, genetic or proteomic profile, genetic or proteomic characteristic, response to previous treatment, weight, height, medical diagnosis, familial background, results of one or more medical tests, ethnic background, body mass index, age, presence or absence of at least one disease or condition, species, ethnicity, race, allergies, gender, presence or absence of at least one biological, chemical, or therapeutic agent in the subject, pregnancy status, lactation status, medical history, or blood condition.

20. The system of claim 14, wherein the at least one protease includes one or more cysteine proteases.

21. The system of claim 20, wherein the at least one protease includes Cathepsin K.

22. The system of claim 14, wherein the at least one protease includes one or more serine proteases.

23. The system of claim 22, wherein the at least one protease includes one or more of PfSUB1, PfSUB2, DPAP1, DPAP2, or DPAP3.

24. The system of claim 22, wherein the at least one protease modulates the activity of one or more of SERA1, SERA2, SERA3, SERA4, SERA5, SERA6, SERA7, or SERA8.

25. The system of claim 24, wherein the at least one protease inhibits the activity of one or more of SERA1, SERA2, SERA3, SERA4, SERA5, SERA6, SERA7, or SERA8.

26. The system of claim 14, wherein the at least one proteasome includes 26S Proteasome.
